(19) United States
(12) Patent Application Publication
(10) Pub. No.: US 2013/0236567 A1
(43) Pub. Date: Sep. 12, 2013

(54) GENE EXPRESSION SIGNATURE AS A PREDICTOR OF CHEMOTHERAPEUTIC RESPONSE IN BREAST CANCER

(71) Applicants: Katherine J. Martin, Belmont, MA (US); David Magee, Collegeville, PA (US); Marcia V. Fournier, Collegeville, PA (US)

(72) Inventors: Katherine J. Martin, Belmont, MA (US); David Magee, Collegeville, PA (US); Marcia V. Fournier, Collegeville, PA (US)

(21) Appl. No.: 13/701,983

(22) PCT Filed: Jun. 6, 2011
(86) PCT No.: PCT/US11/39325

§ 371 Date: May 13, 2013

Related U.S. Application Data
(60) Provisional application No. 61/351,385, filed on Jun. 4, 2010.
(60) Provisional application No. 61/441,554, filed on Feb. 10, 2011.

(51) Int. Cl. C12Q 1/68; G06N 5/02; A61K 31/704; A61K 31/337; A61K 33/24

(52) U.S. Cl. CPC C12Q 1/6886; A61K 31/337; A61K 33/24; A61K 31/704; G06N 5/02
USPC 424/649; 506/16; 506/9; 514/449; 514/34; 706/46

(57) ABSTRACT

Disclosed are methods and compositions for determining and/or predicting a response to a therapy, especially a cancer therapy, including chemotherapy. Specifically, the disclosure provides profiles of a set of marker genes in breast cancers from patients who were known to have responded or not responded to a chemotherapy for predicting response to the same therapy including different combination of chemotherapy in a patient diagnosed with breast cancer. The disclosure further provides computer complemented methods for the prediction based on genetic profiles as well as different clinical parameters. Furthermore, the disclosure provides kits for performing the method disclosed.

### CROSS REFERENCE

This application claims priority to U.S. Provisional Application No. 61/351,385, filed on Jun. 4, 2010, and U.S. Provisional Application No. 61/441,554, filed on Feb. 10, 2011, which are hereby incorporated by reference in their entireties.

### GOVERNMENT INTERESTS

Not Applicable

### PARTIES TO A JOINT RESEARCH AGREEMENT

Not Applicable

### INCORPORATION BY REFERENCE OF MATERIAL SUBMITTED ON A COMPACT DISC

Not Applicable

### BACKGROUND

Not applicable

### BRIEF SUMMARY OF THE INVENTION

Some embodiments described herein are directed to methods for predicting response to chemotherapy in a patient diagnosed with breast cancer. In some embodiments, the method may comprise determining a genetic profile of a set of marker genes in a breast tumor; and comparing the genetic profile of a set of marker genes to a predetermined breast cancer genetic profile of the marker genes, in which a tumor whose genetic profile of a set of marker genes matches the predetermined breast cancer genetic profile of the marker genes is predicted to respond to a treatment.

In another embodiment, the methods for predicting response to chemotherapy in a patient diagnosed with breast cancer may take into account clinical parameters relating to the patient and the tumor. In some embodiments the clinical parameters may comprise, but not limited to, ER-status, HER2 status, patient age, patient race, tumor size, tumor grade, node status and combinations thereof. The clinical parameters alone or combined with the genetic profile of the set of marker genes, such that the clinical parameters alone, or the combination of marker genes and clinical parameters is used to predict a patient's response to a treatment.

In some embodiments, the method may comprise the application of a statistical model (i.e. an algorithm or formula), that has been derived by applying a statistical classification approach, comprising but not limited to logistic regression, to a series of predetermined genetic profiles of the marker genes in breast cancers and/or clinical parameters from patients who were known to have responded or not responded to chemotherapy. The statistical model is used to interpret the genetic profile of the set of marker genes and the clinical parameters of the patient, either alone or in combination, to produce a numerical result, such that a result above a specified cut-off predicts responsiveness and a result below a specified cut-off predicts non-responsiveness.

In some embodiments, the set of marker genes may comprise, but is not limited to, EPHA2, FGFBP1, TNFRSF6B, FOXM1, CDKN3, RRM2, CKS2, ASPM, AURKA, CEP55, TRIP13, TUBG1, ZWILCH, VRK1, SERPINE2, ODC1, CAPRIN2, ACTB, ACTN1, CAPG, DUSP4 and EIF4A1. In some embodiments, the genetic profile of a set of marker genes may comprise, but not limited to, the genetic profile of RNA, cDNA, protein, microRNA, fragments of RNA, fragments of cDNA, fragments of protein and fragments of microRNA of the set of marker genes.

In some embodiments, matching of the genetic profile of the patient's tumor with a predetermined genetic profile may comprise, but is not limited to a Person Product Moment correlation coefficient (r) of greater than 0.70, r of greater than 0.75, r of greater than 0.85, r of greater than 0.90, r of greater than 0.95, r of greater than 0.99 and r of 1.

In some embodiments, the test will predict if a patient who has an ER− subtype breast cancer will respond to combination chemotherapy with taxol, 5-fluorouracil, anthracycline and cyclophosphamide (TFAC). ER-negative tumors do not express the receptor for the hormone estrogen.

In some embodiments, the test will predict if a patient who has an ER+ subtype breast cancer is responsive to TFAC combination chemotherapy. ER-positive tumors express the receptor for the hormone estrogen.

In some embodiments, the test will predict if a patient who has a luminal A subtype breast cancer is responsive to TFAC combination chemotherapy. Luminal A breast cancers are identified by expression profiling and typically express the receptor for the hormone estrogen and low levels of proliferation related genes.

In some embodiments, the test will predict if a patient who has a luminal B subtype breast cancer is responsive to TFAC combination chemotherapy. Luminal B breast cancers are identified by expression profiling and typically express the receptor for the hormone estrogen and high levels of proliferation related genes.

In some embodiments, the test will predict if a patient who has a basal-like subtype breast cancer is responsive to TFAC combination chemotherapy. Basal-like breast cancers are identified by expression profiling and typically express neither ER, the receptor for the hormone estrogen, nor HER2, the receptor for human epidermal growth factor.

In some embodiments, the test will predict if a patient who has the HER2-positive subtype of breast cancer is responsive to TFAC combination chemotherapy. HER2— positive breast cancers are identified by expression profiling and typically express HER2, the receptor for human epidermal growth factor.

Some embodiments described herein are directed to a kit for testing therapeutic sensitivity of breast cancer. The kit may comprise a means for identifying a genetic profile of a set of marker genes in a sample having probes to a specific set of genes associated with breast cancer; and labels, reagents, and other materials or instructions for preparing reagents and other materials necessary to develop a genetic profile of a set of marker genes.

Some embodiments described herein are directed methods of predicting therapeutic response of a patient to a chemotherapeutic. In some embodiments, the method may comprise determining a genetic profile from a set of marker genes; determining a set of clinical parameters; and applying at least one statistical model based on the genetic profile and the clinical parameters, wherein result of the statistical model predicts the therapeutic response of the patient to a chemotherapeutic. In some embodiments, the clinical parameter may comprise, but not limited to, ER-status, HER2 status, patient age, patient race, tumor size, tumor grade, node status and combinations thereof. In some embodiments, the statistical model may comprise, but not limited to, logistic regression, cluster analysis and combinations thereof.

In some embodiments, the set of marker genes may comprise, but is not limited to, EPHA2, FGFBP1, TNFRSF6B, FOXM1, CDKN3, RRM2, CKS2, ASPM, AURKA, CEP55, TRIP13, TUBG1, ZWILCH, VRK1, SERPINE2, ODC1, CAPRIN2, ACTB, ACTN1, CAPG, DUSP4 and EIF4A1.

Embodiments of the present invention are directed to methods for predicting therapeutic response to breast cancer comprising: isolating genetic material from a diseased tissue sample of a patient with breast cancer; developing a genetic profile from the marker genes; and predicting therapeutic response in said patient based on said genetic profile and providing treatment to a patient whose expression profile matches or nearly matches a predetermined profile that indicates that a patient will respond to the treatment.

In certain embodiments, the genetic profile from the marker genes is referred to as a 3D Signature. In certain embodiment, the 3D signature is simply referred to as “signature”. Unlike most cancer signatures that have been selected by using supervised methods and a specific patient training set, the 3D Signature was selected using a cell culture model that accurately recapitulates the normal process of breast acini formation and growth arrest. Since it is not linked to a particular patient set, the signature has the potential to more accurately classify diverse patient subsets than traditionally discovered signatures.

In yet another embodiment, the 3D signature can be applied to predict chemotherapy response for breast cancers of different subtypes. While the signature can be applied to a mixed set of patients that includes all subtypes of breast cancers or to a single patient whose breast cancer subtype is not known, it can also be applied to predict chemotherapy response for a homogeneous set of patients of one subtype or a single patient whose breast cancer subtype is known. The breast cancer subtypes include, but are not limited to, ER-positive, ER-negative, HER2-positive, triple negative, luminal A, luminal B, and basal-like.

In another embodiment, the 3D signature is applied to accurately predict response in different subtypes by the development of a series of models (i.e. algorithms or formulas each with individual and specific patterns of gene weighting) where all of the different models are based on the same gene signature. These models are developed by using a statistical classification approach, including but not limited to logistic regression. The models are developed using a dataset consisting of gene expression patterns obtained from patients with a predetermined breast cancer subtype. The 3D signature includes a sufficient number of genes and has sufficient discrimination power for the development of accurate models for all and each subtype of breast cancer.

Another embodiment of the present invention is directed to a kit for testing therapeutic sensitivity of diseased tissue comprising: a means for identifying the expression profile of a tissue sample having probes to a specific set of genes or proteins associated with the disease; and labels, reagents, and other materials or instructions for labeling and preparing reagents and other materials necessary to develop a expression profile of one or more marker genes.

In some embodiments, the present invention provides methods for predicting a response to a cancer treatment. In some embodiments, the patient has cancer. In some embodiments, the patient is suspected of, or diagnosed, with cancer. In some embodiments, the method comprises transforming a data set to yield a predictive score, wherein the predictive score predicts whether or not a cancer will respond to a treatment. In some embodiments, the data is transformed by an interpretation function. In some embodiments, the data set is an expression data set.

In some embodiments, a method comprises obtaining a dataset associated with a sample derived from a patient diagnosed with cancer. In some embodiments, the dataset comprises expression data for at least one marker. In some embodiments, the at least one marker selected from the group consisting of FLJ10517, HCAP-G, CDKN3, STK6, FOXM1, FLJ10540, TNFRSF6B, HBP17, C1 QDC1, TUBG1, FLJ10036, RRM2, ACTB, ACTN1, EPHA2, TRIP13, CKS2, VRK1, DUSP4, EIF4A1, SERPINE2, and ODC1. In some embodiments, a data set comprises at least one clinical factor.

In some embodiments, a method comprises determining a predictive score from the dataset using an interpretation function, wherein the predictive score is predictive of the response to the cancer treatment.

In some embodiments, the cancer is brain cancer (gliomas), glioblastomas, leukemias, breast, Wilm's tumor, Ewing's sarcoma, Rhabdomyosarcoma, ependymoma, medulloblastoma, colon, head and neck, kidney, lung, liver, melanoma, ovarian, pancreatic, prostate, sarcoma, osteosarcoma, giant cell tumor of bone, thyroid, Lymphoblastic T cell leukemia, Chronic myelogenous leukemia, Chronic lymphocytic leukemia, Hairy-cell leukemia, acute lymphoblastic leukemia, acute myelogenous leukemia, Chronic neutrophilic leukemia, Acute lymphoblastic T cell leukemia, Plasmacytoma, Immunoblastic large cell leukemia, Mantle cell leukemia, Multiple myeloma Megakaryoblastic leukemia, multiple myeloma, Acute megakaryocytic leukemia, promyelocytic leukemia, Erythroleukemia, malignant lymphoma, hodgkins lymphoma, non-hodgkins lymphoma, lymphoblastic T cell lymphoma, Burkitt's lymphoma, follicular lymphoma, neuroblastoma, bladder cancer, urothelial cancer, lung cancer, vulval cancer, cervical cancer, endometrial cancer, renal cancer, mesothelioma, esophageal cancer, salivary gland cancer, hepatocellular cancer, gastric cancer, nasopharangeal cancer, buccal cancer, cancer of the mouth, GIST (gastrointestinal stromal tumor), or testicular cancer, and the like.

In some embodiments, the method is computer implemented. In some embodiments, the determining step is determined by a computer processor. In some embodiments, the dataset comprises the expression data and the at least one clinical factor.

In some embodiments, the predictive score is compared to a score derived from a sample from a patient with cancer that was known to have responded or not responded to chemotherapy, wherein a sample whose score matches the predetermined predictive of sample derived from a patient that responded to treatment the patient diagnosed with cancer is predicted to respond to the cancer treatment, or wherein a sample whose score matches the predetermined predictive of sample derived from a patient that did not respond to treatment the patient diagnosed with cancer is predicted to not to respond to the cancer treatment.

In some embodiments, the response that is predicted is a complete response, partial response or no response. In some embodiments, the response that is predicted is a pathological complete response. In some embodiments, the response that is predicted is at least 5, 7, or 10 year survival. In some embodiments, the survival and/or the response is relapse-free.

In some embodiments, the interpretation function is based upon a predictive model. In some embodiments, the predictive model is a logistical regression model. In some embodiments, the logistic regression model is applied to the dataset to interpret the dataset to produce the predictive score, wherein a predictive score above a specified cut-off value predicts responsiveness and a predictive score below a specified cut-off predicts non-responsiveness.

In some embodiments, the patient diagnosed with cancer has an ER-positive cancer, an ER-negative cancer, a cancer characterized as Luminal A, Luminal B, or a cancer characterized as basal-like. In some embodiments, the patient diagnosed with cancer has a triple-negative cancer.

In some embodiments, the response is a response for a cancer treatment. In some embodiments, the treatment is adjuvant chemotherapy. In some embodiments, the treatment is neoadjuvant chemotherapy. In some embodiments, the cancer is predicted to response or not respond to a cancer treatment, such as, but not limited to, a breast cancer treatment.

In some embodiments, the sample that is tested or analyzed comprises extracted RNA. In some embodiments, the sample comprises RNA extracted from breast epithelial cells. In some embodiments, the sample comprises RNA extracted from breast tumor cells.

In some embodiments, systems are provide for predicting a response to a cancer treatment. In some embodiments, a system for predicting a response to a cancer treatment comprises a storage memory for storing a dataset associated with a sample. In some embodiments, the sample is obtained from a subject. In some embodiments, the dataset comprises expression data for at least one marker. In some embodiments, the at least one marker selected from the group consisting of FLJ10517, HCAP-G, CDKN3, STK6, FOXM1, FLJ10540, TNFRSF6B, HBP17, C1QDC1, TUBG1, FLJ10036, RRM2, ACTB, ACTN1, EPHA2, TRIP13, CKS2, VRK1, DUSP4, EIF4A1, SERPINE2, and ODC1. In some embodiments, a system comprises a data set that comprises at least one clinical factor. In some embodiments, the system comprises a processor communicatively coupled to the storage memory for determining a score with an interpretation function wherein the score is predictive of response to a cancer treatment in a subject. In some embodiments, the subject has been diagnosed with cancer or is known to or suspected of having cancer.

In some embodiments, kits are provided for predicting a response to a cancer treatment in a subject. In some embodiments, a kit for predicting a response to a cancer treatment in a subject comprises one or more reagents for determining from a sample obtained from a subject expression data for at least one marker selected from the group consisting of FLJ10517, HCAP-G, CDKN3, STK6, FOXM1, FLJ10540, TNFRSF6B, HBP17, C1QDC1, TUBG1, FLJ10036, RRM2, ACTB, ACTN¹, EPHA2, TRIP13, CKS2, VRK1, DUSP4, EIF4A1, SERPINE2, and ODC1. In some embodiments, the kit comprises instructions for using the one or more reagents to determine expression data from the sample, wherein the instructions include instructions for determining a score from the dataset wherein the score is predictive of response to the cancer treatment. In some embodiments, the data comprises at least one clinical factor. In some embodiments, the kit is configured to determine whether a cancer will respond to an adjuvant and/or a neoadjuvant therapy. In some embodiments, the kit is configured to determine a response to a cancer treatment as described herein.

In some embodiments, a method for predicting a response to a cancer treatment in a patient diagnosed with cancer comprises isolating a sample of the cancer from the patient diagnosed with cancer; obtaining a dataset associated with a sample derived from a patient diagnosed with cancer, wherein the dataset comprises expression data for at least one marker selected from the group consisting of FLJ10517, HCAP-G, CDKN3, STK6, FOXM1, FLJ10540, TNFRSF6B, HBP17, C1QDC1, TUBG1, FLJ10036, RRM2, ACTB, ACTN1, EPHA2, TRIP13, CKS2, VRK1, DUSP4, EIF4A1, SERPINE2, and ODC1 and at least one clinical factor; and determining a predictive score from the dataset using an interpretation function, wherein the interpretation function comprises is based upon a predictive model, wherein the predictive model is a logistical regression model, wherein the logistical regression model is applied to the dataset to interpret the dataset to produce the predictive score, and wherein a predictive score above a specified cut-off value predicts responsiveness and a predictive score below a specified cut-off predicts non-responsiveness.

### BRIEF DESCRIPTION OF THE DRAWINGS

For a fuller understanding of the nature and advantages of the present invention, reference should be made to the following detailed description taken in connection with the accompanying drawings, in which:

FIG. 1 illustrates that a 3D Signature was discovered by gene expression analysis of cultured breast epithelial cells grown in a 3D model of laminin-rich extracellular matrix (lrECM). Genes down regulated during acini formation and growth arrest were identified and then tested for their ability to classify patients by long-term prognosis in three unrelated sets of breast cancer patients.

FIG. 2 shows that the 3D Signature accurately predicted clinical breast cancer outcome. In a retrospective analysis, the 3D signature was prognostic in three independent, previously published datasets that totaled over 700 breast cancer patients.

FIG. 3 shows the division of the 278 breast cancer patients of the microarray dataset of Hess, et al., 2006 into molecular classes. Gene expression levels for 263 intrinsic genes are shown. Red=over expression, blue=under expression.

FIG. 4 shows the implications of using the 3D gene Signature for breast cancer patients in responding to chemotherapy in order to assess further treatment options.

FIG. 5 illustrates that the 22 gene signature includes functional gene classes including cell cycle, motility, and angiogenesis.

FIG. 6 illustrates prediction of response to taxol combination chemotherapy by the 22 gene signature in multiple subclasses of breast cancer patients using logistic regression.

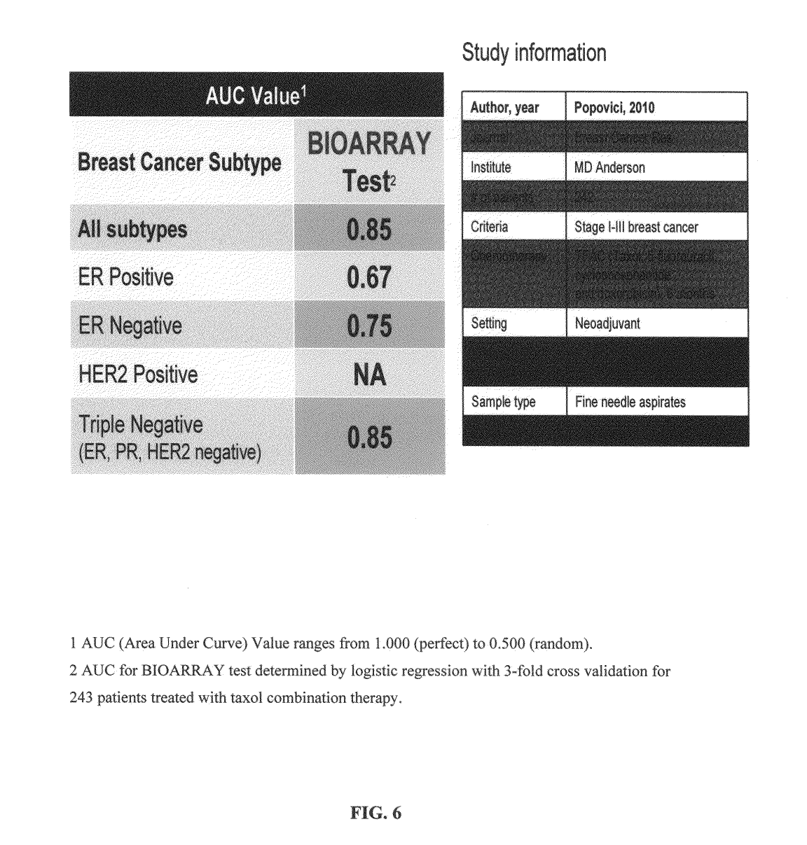

FIG. 7 illustrates comparison of taxol combination (TFAC) versus non-taxol combination (FAC) chemotherapy response in breast cancer using logistic regression with the 22 gene signature. The objective of this experiment was to test if the 22 gene signature model that predicts TFAC response also predicts FAC response. Microarray data from a randomized trial with two arms, TFAC and FAC, were collected at MD Anderson Cancer Center (Tabchy et al 2010). The 22 gene signature was optimized by sequentially omitting from the analysis genes with lowest p values. A. Discovery logistic regression results from 37 ER-negative samples from patients treated with TFAC. B. Discovery logistic regression results from 42 ER-negative samples from patients treated with FAC. These results indicate that expression levels of the 22 genes allow accurate prediction of response to both TFAC and FAC, though the optimized models differ markedly. Only 50% of optimized genes are overlapping and for these overlapping genes, odds ratio vary greatly between the two datasets. Hence, the 22 gene signature has the potential to accurately predict response to both taxol combination chemotherapy and non taxol combination chemotherapy by using logistic regression different models.

FIG. 8 illustrates comparison of discovery logistic regression output results (using MedCalc software) to assess ability of the 22 gene signature to predict response to taxol combination versus single agent cisplatin chemotherapy response in breast cancer using logistic regression. This study used a simplified version of logistic regression, where AUCs are calculated on the training set and no test sets or cross validation is applied. The objective of this experiment was to test if the 22 gene model that predicts TFAC response also predicts cisplatin response. Microarray data for the 24 biopsy samples from patients subsequently treated with neoadjuvant cisplatin were collected at the Dana Farber Cancer Institute (Silver et al 2010). For each analysis, the 22 gene signature was optimized by sequentially omitting from the analysis genes with lowest p values. A. Discovery logistic regression results from 243 samples from patients treated with TFAC (Popovici et al 2010). Resulting AUC of 0.834 indicates a very good prediction test that is statistically significant (p<0.0001). B. Discovery logistic regression results from 24 samples from patients treated with cisplatin (Silver et al 2010). The resulting AUC of 1.0 indicates a perfect test, though the number of samples was to low to achieve statistical significance (p=0.4823). C. Discovery logistic regression analysis of the combined datasets of TFAC and cisplatin was performed to test whether the same model was applicable to both datasets. An AUC of 0.806 was obtained, which is less than 0.834 obtained for the TFAC dataset alone. Though samples number were not large enough to obtain significance, this result suggests that expression levels of the 22 genes allowed the prediction of response to both cisplatin and TFAC, but through different models.

### DETAILED DESCRIPTION

Before compositions and methods provided herein are described, it is to be understood that this invention is not limited to the particular processes, compositions, or methodologies described, as these may vary. It is also to be understood that the terminology used in the description is for the purpose of describing some embodiments, and is not intended to limit the scope of the present invention. All publications mentioned herein are incorporated by reference in their entirety to the extent to support the present invention.

It must be noted that, as used herein and in the appended claims, the singular forms “a”, “an” and “the” include plural reference unless the context clearly dictates otherwise. Unless defined otherwise, all technical and scientific terms used herein have the same meanings as commonly understood by one of ordinary skill in the art. Although any methods similar or equivalent to those described herein can be used in the practice or testing of embodiments of the present invention, the preferred methods are now described. All publications and references mentioned herein are incorporated by reference. Nothing herein is to be construed as an admission that the invention is not entitled to antedate such disclosure by virtue of prior invention.

As used herein, the term “about” means plus or minus 10% of the numerical value of the number with which it is being used. Therefore, about 50% means in the range of 45%-55%. Additionally, in phrase “about X to Y,” is the same as “about X to about Y,” that is the term “about” modifies both “X” and “Y.”

“Optional” or “optionally” may be taken to mean that the subsequently described structure, event or circumstance may or may not occur, and that the description includes instances where the event occurs and instances where it does not. “Administering” when used in conjunction with a therapeutic means to administer a therapeutic directly into or onto a target tissue or to administer a therapeutic to a patient whereby the therapeutic positively impacts the tissue to which it is targeted. “Administering” a composition may be accomplished by oral administration, injection, infusion, absorption or by any method in combination with other known techniques.

The term “target”, as used herein, refers to the material for which either deactivation, rupture, disruption or destruction or preservation, maintenance, restoration or improvement of function or state is desired. For example, diseased cells, pathogens, or infectious material may be considered undesirable material in a diseased subject and may be a target for therapy.

Generally speaking, the term “tissue” refers to any aggregation of similarly specialized cells which are united in the performance of a particular function.

The term “improves” is used to convey that the present invention changes either the appearance, form, characteristics and/or physical attributes of the tissue to which it is being provided, applied or administered. “Improves” may also refer to the overall physical state of an individual to whom an active agent has been administered. For example, the overall physical state of an individual may “improve” if one or more symptoms of a disorder or disease are alleviated by administration of an active agent.

As used herein, the term “therapeutic” or “therapeutic agent” means an agent utilized to treat, combat, ameliorate or prevent an unwanted condition or disease of a patient. In certain embodiments, a therapeutic or therapeutic agent may be a composition including at least one active ingredient, whereby the composition is amenable to investigation for a specified, efficacious outcome in a mammal (for example, without limitation, a human). Those of ordinary skill in the art will understand and appreciate the techniques appropriate for determining whether an active ingredient has a desired efficacious outcome based upon the needs of the artisan.

The terms “therapeutically effective amount” or “therapeutic dose” as used herein are interchangeable and may refer to the amount of an active agent or pharmaceutical compound or composition that elicits a biological or medicinal response in a tissue, system, animal, individual or human that is being sought by a researcher, veterinarian, medical doctor or other clinician. A biological or medicinal response may include, for example, one or more of the following: (1) preventing a disease, condition or disorder in an individual that may be predisposed to the disease, condition or disorder but does not yet experience or display pathology or symptoms of the disease, condition or disorder, (2) inhibiting a disease, condition or disorder in an individual that is experiencing or displaying the pathology or symptoms of the disease, condition or disorder or arresting further development of the pathology and/or symptoms of the disease, condition or disorder, and (3) ameliorating a disease, condition or disorder in an individual that is experiencing or exhibiting the pathology or symptoms of the disease, condition or disorder or reversing the pathology and/or symptoms experienced or exhibited by the individual.

The term “treating” may be taken to mean prophylaxis of a specific disorder, disease or condition, alleviation of the symptoms associated with a specific disorder, disease or condition and/or prevention of the symptoms associated with a specific disorder, disease or condition.

The term “patient” generally refers to any living organism to which to compounds described herein are administered and may include, but is not limited to, any non-human mammal, primate or human. Such “patients” may or may not be exhibiting the signs, symptoms or pathology of the particular diseased state.

As used herein, a “kit” refers to one or more diagnostic or prognostic assays or tests and instructions for their use. The instructions may consist of product insert, instructions on a package of one or more diagnostic or prognostic assays or tests, or any other instruction. In some embodiments, a kit comprises components to perform the assays or tests. For example, the kit can comprise primers or other reagents to be used in the analysis of a gene's expression. The kit can also comprise enzymes, such as polymerases or reverse transcriptases, to be used in the assays or tests.

The terms “marker” or “markers” encompass, without limitation, lipids, lipoproteins, proteins, cytokines, chemokines, growth factors, peptides, nucleic acids, genes, and oligonucleotides, together with their related complexes, metabolites, mutations, variants, polymorphisms, modifications, fragments, subunits, degradation products, elements, and other analytes or sample-derived measures. A marker can also include mutated proteins, mutated nucleic acids, variations in copy numbers, and/or transcript variants, in circumstances in which such mutations, variations in copy number and/or transcript variants are useful for generating a predictive model, or are useful in predictive models developed using related markers (e.g., non-mutated versions of the proteins or nucleic acids, alternative transcripts, etc.). In some embodiments, the “3D-signature” comprises one or more markers as disclosed herein. The “3D-Signature,” in some embodiments, comprises at least 1, 2, 3, 4, 5, 6, 7, 8, 9, 10, 11, 12, 13, 14, 15, 16, 17, 81, 19, 20, 21, 22, 10-20, 15-20, 20-22, or 1-20 markers.

As used herein, the term “triple-negative” as applied to a cancer refers to a cancer that is ER (estrogen receptor)-negative, PR (progesterone receptor)-negative, and Her2-negative).

As used herein, the term “predictive score” is a score that is calculated (e.g. determined) according to a method including those methods described herein. The predictive score can be used to predict a cancer's response to a cancer treatment in general or to a specific type of treatment. The predictive score can also be for a particular type of cancer. The predictive score can be compared to a cut-off value (as, for example, described herein) to determine whether or not a cancer will respond to a treatment.

The methods disclosed herein can be used to predict a response to a cancer treatment. The cancer treatment can be any treatment including, but not limited, to the treatments and therapies described herein. Additionally, the methods can be used to predict the response of any cancer. Examples of cancers include solid and non-solid cancer. Examples of cancers include, but are not limited to, brain (gliomas), glioblastomas, leukemias, breast, Wilm's tumor, Ewing's sarcoma, Rhabdomyosarcoma, ependymoma, medulloblastoma, colon, head and neck, kidney, lung, liver, melanoma, ovarian, pancreatic, prostate, sarcoma, osteosarcoma, giant cell tumor of bone, thyroid, Lymphoblastic T cell leukemia, Chronic myelogenous leukemia, Chronic lymphocytic leukemia, Hairy-cell leukemia, acute lymphoblastic leukemia, acute myelogenous leukemia, Chronic neutrophilic leukemia, Acute lymphoblastic T cell leukemia, Plasmacytoma, Immunoblastic large cell leukemia, Mantle cell leukemia, Multiple myeloma Megakaryoblastic leukemia, multiple myeloma, Acute megakaryocytic leukemia, promyelocytic leukemia, Erythroleukemia, malignant lymphoma, hodgkins lymphoma, non-hodgkins lymphoma, lymphoblastic T cell lymphoma, Burkitt's lymphoma, follicular lymphoma, neuroblastoma, bladder cancer, urothelial cancer, lung cancer, vulval cancer, cervical cancer, endometrial cancer, renal cancer, mesothelioma, esophageal cancer, salivary gland cancer, hepatocellular cancer, gastric cancer, nasopharangeal cancer, buccal cancer, cancer of the mouth, GIST (gastrointestinal stromal tumor), testicular cancer, any combination thereof, and the like. The cancer can also be a patient who has been diagnosed with cancer. The cancer can also refer to a patient who has had cancer and has either responded or not responded to a treatment.

As used herein, the term “sample” can refer to a single cell or multiple cells or fragments of cells or an aliquot of body fluid, taken from a subject. In some embodiments the sample is a biological sample. In some embodiments, the sample is a fixed, paraffin-embedded, fresh, or frozen tissue sample. In some embodiments, the sample is derived from a fine needle, core, or other type of biopsy. The sample can, for example, be obtained from a subject by, but not limited to, venipuncture, excretion, biopsy, needle aspirate, lavage sample, scraping, surgical incision, or, any combination thereof, and the like.

In some embodiments, the bodily fluid is blood, urine, saliva, and the like. In some embodiments, the cell is a cancerous cell or a normal cell. In some embodiments, the tissue is a cancerous tissue. In some embodiments, the tissue is a normal tissue. In some embodiments, the sample is a tumor or cells derived from a tumor. In some embodiments, the sample is a cell derived from normal tissue. In some embodiments, the sample is hair or cells that have been derived from hair. The sample is any biological product that can be tested and form which nucleic acid material can be derived from. In some embodiments, the cell is a blood cell, such as but not limited to, white blood cells. In some embodiments, the cell is a breast epithelial cell. The breast epithelial cell can be a cancerous cell or a non-cancerous cell. In some embodiments, the sample comprises cancerous and non-cancerous cells, tissues, fluids, and the like. In some embodiments, the sample is free of non-cancerous cells and tissues. In some embodiments, the sample is free of cancerous cells and tissues. A “cancerous fluid” is a fluid derived from a subject that has cancer. In some embodiments, the sample is electronic data. In some embodiments, the sample comprises expression data.

As used herein, the term “expression data” refers to expression levels of one or more markers. The expression data can comprise the expression levels of RNA, mRNA, protein, and the like. The expression levels can be quantified. The quantification can be based upon absolute amounts or be based on a comparison to a standard.

The expression data can be measured for the markers described herein or sequences that are homologous to the sequences described herein. In some embodiments, the sequence or probe is at least 85, 90, 91, 92, 93, 94, 95, 96, 97, 98, 99% identical to the sequences described herein. In some embodiments, the sequence is from about 85-99, 90-99, 92-99, 93-99, 94-99, 95-99, 96-99, 97-99, or 98-99% identical to sequence described herein. In some embodiments, the sequence comprises at least or exactly 1, 2, 3, 4, or 5 mutations. The mutation can be an insertion, silent, deletion, point mutation, or any combination thereof, and the like.

Nucleic acid molecules or sequences can also be referred to as being substantially complementary to another sequence. “substantially complementary” refers to a nucleic acid sequence that is at least 70%, 80%, 85%, 90% or 95% complementary to at least a portion of a reference nucleic acid sequence or to the entire sequence. By “complementarity” or “complementary” is meant that a nucleic acid can form hydrogen bond(s) with another nucleic acid sequence by either traditional Watson-Crick or other non-traditional types of interaction. In reference to the nucleic molecules, the binding free energy for a nucleic acid molecule with percent complementarity indicates the percentage of contiguous residues in a nucleic acid molecule that can form hydrogen bonds (e.g., Watson-Crick base pairing) with a second nucleic acid sequence (e.g., 5, 6, 7, 8, 9, 10 out of 10 being 50%, 60%, 70%, 80%, 90%, and 100% complementary). “Perfectly complementary” means that all the contiguous residues of a nucleic acid sequence will hydrogen bond with the same number of contiguous residues in a second nucleic acid sequence.

By “substantially identical” is meant a polypeptide or nucleic acid exhibiting at least 90%, 95%, or 99% identity to a reference sequence (e.g. nucleic acid sequence). For nucleic acids, by “substantially identical” can be interchanged with “substantially complementary.” For nucleic acids, the length of comparison sequences can be at least 10 15, 20, 25, 30 nucleotides. For nucleic acids, the length of comparison sequences can be about 5-30, about 10-25, about 10-20, about 15-25, about 20-30, about 20-25, about 25-20 nucleotides.

The term “identity” or is used herein to describe the relationship of the sequence of a particular nucleic acid molecule or polypeptide to the sequence of a reference molecule of the same type. For example, if a polypeptide or nucleic acid molecule has the same amino acid or nucleotide residue at a given position, compared to a reference molecule to which it is aligned, there is said to be “identity” at that position. The level of sequence identity of a nucleic acid molecule or a polypeptide to a reference molecule is typically measured using sequence analysis software with the default parameters specified therein, such as the introduction of gaps to achieve an optimal alignment. Methods to determine identity are available in publicly available computer programs. Computer program methods to determine identity between two sequences include, but are not limited to, the GCG program package (Devereux et al., Nucleic Acids Research 12(1): 387, 1984), BLASTP, BLASTN, and FASTA (Altschul et al., J. Mol. Biol. 215: 403 (1990). The well-known Smith-Waterman algorithm may also be used to determine identity. The BLAST and BLAST2 programs are publicly available from NCBI and other sources (BLAST Manual, Altschul, et al., NCBI NLM NIH

Bethesda, Md. 20894). Searches can be performed in URLs such as http://www.ncbi.nlm.nih.gov/BLAST or http://www.ncbi.nlm.nih.gov/gorf/b12.html (Tatusova et al., FEMS Microbiol. Lett. 174:247-250, 1999). These software programs match similar sequences by assigning degrees of homology to various substitutions, deletions, and other modifications. Conservative substitutions typically include substitutions within the following groups: glycine, alanine; valine, isoleucine, leucine; aspartic acid, glutamic acid, asparagine, glutamine; serine, threonine; lysine, arginine; and phenylalanine, tyrosine. Alternatively, or additionally, two nucleic acid sequences are “substantially identical” if they hybridize under high stringency conditions.

Percent identity and percent complementarity can also be determined electronically, e.g., by using the MEGALIGN program (DNASTAR, Inc. Madison, Wis.). The MEGALIGN program can create alignments between two or more sequences according to different methods, for example, the clustal method. (See, for example, Higgins and Sharp (1988) Gene 73: 237-244.) The clustal algorithm groups sequences into clusters by examining the distances between all pairs. The clusters are aligned pairwise and then in groups. Other alignment algorithms or programs may be used, including FASTA, BLAST, or ENTREZ, FASTA and BLAST, and which may be used to calculate percent similarity. These are available as a part of the GCG sequence analysis package (University of Wisconsin, Madison, Wis.), and can be used with or without default settings. ENTREZ is available through the National Center for Biotechnology Information. In some embodiments, the percent identity of two sequences can be determined by the GCG program with a gap weight of 1, e.g., each nucleotide mismatch between the two sequences (see U.S. Pat. No. 6,262,333). Other techniques for alignment are described in Methods in Enzymology, vol. 266, Computer Methods for Macromolecular Sequence Analysis (1996), ed. Doolittle, Academic Press, Inc., San Diego, Calif., USA. Preferably, an alignment program that permits gaps in the sequence is utilized to align the sequences. The Smith-Waterman is one type of algorithm that permits gaps in sequence alignments (see Shpaer (1997) Methods Mol. Biol. 70: 173-187). Also, the GAP program using the Needleman and Wunsch alignment method can be utilized to align sequences. An alternative search strategy uses MPSRCH software, which runs on a MASPAR computer. MPSRCH uses a Smith-Waterman algorithm to score sequences on a massively parallel computer. This approach improves ability to pick up distantly related matches, and is especially tolerant of small gaps and nucleotide sequence errors.

A “variant” refers to a sequence that is not 100% identical to a sequence described herein. The variant may have the various mutations or levels of identity or complementarity as described herein. In some embodiments, the variant is at least 100% identical over a portion of the sequences described herein. In some embodiments, the portion is from about 10-100, 10-200, 10-300, 10-400, 10-500, 10-600, 50-100, 50-200, 50-300, 50-400, 50-500, 50-600 nucleotides in length. In some embodiments, the portion is at least 10, 20, 30, 40, 50, 60, 70, 80, 90, 100, 200, 300, 400, 500, or 600 nucleotides in length.

In some embodiments, the sequence detected and/or measure has two non-contiguous portions that are 100% identical to a sequence described herein. The non-contiguous portions can be separated by at least 1, 2, 3, 4, 5, 6, 7, 8, 9, or 10 unmatched nucleotides or by at least 1, 2, 3, 4, 5, 6, 7, 8, 9, or 10 nucleotides that create a cap when the sequences are aligned. Methods of alignment are described herein.

Early detection of cancer is vital for patient survival by increasing treatment options. For example, breast cancer ranks as the second leading cause of death among women with cancer in the U.S., and early detection of breast cancer has a significant effect on patient survival, though a portion of patients still may relapse and may develop a more aggressive form of disease. As such, methods of predicting chemotherapy response in a broad range of breast cancer subtypes has become a primary focus of cancer research. Key steps include determining which patients will benefit from standard care therapies and assessing their chances of disease progression. The present invention provides methods for predicting (e.g. determining) a tumor or cancer's chemotherapy response.

Metastasis is a multi-step process during which cancer cells disseminate from the site of primary tumors and establish secondary tumors in distant organs. While established cancer prognostic markers such as tumor size, grade, nodal, and hormone receptor status are useful in predicting survival in large populations, there is a need to develop better prognostic signatures to predict the efficacy of various forms of cancer treatment. A particular benefit would be the identification of patients with good prognoses that are being treated with chemotherapies. The advent of gene expression technologies has greatly aided the identification of molecular signatures with value for tumor classification and prognosis prediction.

Several studies have been performed to identify predictive gene-signatures for breast cancer and have been shown to be of value in evaluating the clinical prognosis in breast cancer. However, most of these gene-signatures have been selected using supervised methods applied to training sets of about 50-100 patients, and then confirmed in larger related sets ranging from 100-300 patients. Furthermore, the individual genes that make up the signatures identified in different studies show surprisingly little overlap, and investigations addressing this lack of overlap have found that predictive signatures are highly dependent on the specific set of patients that make up the training set. For example, two predictive signatures for breast cancer identified by microarray analysis have been developed into clinical multi-gene panel tests. MammaPrint® is composed of 70 genes which were identified by analyzing the large NKI dataset of van de Vijver, et al. Unfortunately, subsequent analysis found that the gene-signature used in the MammaPrint® panel did not predict outcome as well in an independent dataset, and several clinical trials are ongoing to test the utility of this prognostic gene-signature test.

Even though these gene-signatures have been helpful in identifying patients at risk of some types of cancer, they have provided limited information on which genes are particularly relevant to cancer biology since all genes included in a gene-signature cannot be key biological players in cancer progression and response to therapy. Moreover, these gene-signatures provide little information regarding which type of treatment will be most effective in for treating an individual exhibiting a particular expression pattern. The present invention overcomes these deficiencies as well as others.

Various embodiments of the invention are directed to tests for therapeutic sensitivity (i.e., whether a tumor will respond to treatment) by identifying a number of genes whose expression patterns are modified as a result of cancer, and other embodiments of the invention are directed to methods for performing such tests. The term “tests” can also be referred to as a clinical test or other similar wording. In some embodiments, the therapeutic sensitivity or response that is predicted is a partial response. In some embodiments, the therapeutic sensitivity or response that is predicted is a pathological complete response. In some embodiments, the response is a pathological complete response. An example of a pathological complete response refers to the absence of any residual tumor upon histological exam. In some embodiments, the predicted response is at least 5, 7, or 10 year survival. In some embodiments, the survival is relapse-free. in some embodiments, the survival is not relapse free. A partial response can refer to a response where the tumor or amount of cancer in the subject has decreased but the tumor or cancer can still be detected. For example, the tumor size may shrink in size but still be detectable. This can be classified as a partial response. A non-limiting example of a pathological complete response is described in (Bonadonna et al, (1998) Primary chemotherapy in operable breast cancer: eight-year experience at the Milan Cancer Institute. J Clin Oncol 16: 93-100; Fisher et al. (1998) Effect of preoperative chemotherapy on the outcome of women with operable breast cancer. J Clin Oncol 16: 2672-2685; and Kuerer et al., (1999) Clinical course of breast cancer patients with complete pathologic primary tumour and axillary lymph node response to doxorubicin-based neoadjuvant chemotherapy. J Clin Oncol 17: 460-469, each of which is hereby incorporated by reference in its entirety.

Yet another embodiment of the invention is directed to predicting a chemotherapeutic response in breast cancer by identifying a number of genes whose expression patterns are modified as a result of therapy. In a preferred embodiment a “3D gene Signature” is used to predict the efficacy of treatment. Unlike most cancer signatures that have been selected by using supervised methods and a specific patient training set, the 3D Signature was selected using a cell culture model that accurately recapitulates the normal process of breast acini formation and growth arrest. Since this process is not linked to a particular patient set, the 3D Signature more accurately classifies diverse patient subsets than traditionally discovered signatures. The “3D signature” refers to a gene signature that is derived from a tumor or non-tumor sample that is grown in an ex vivo environment and can grow three dimensionally, as opposed to other methods of cell culture, which only allow cells to grow in two dimensions and only create a monolayer. In a 3D environment, the cells can grow to form clusters that are more representative of tissue and cell growth in vivo.

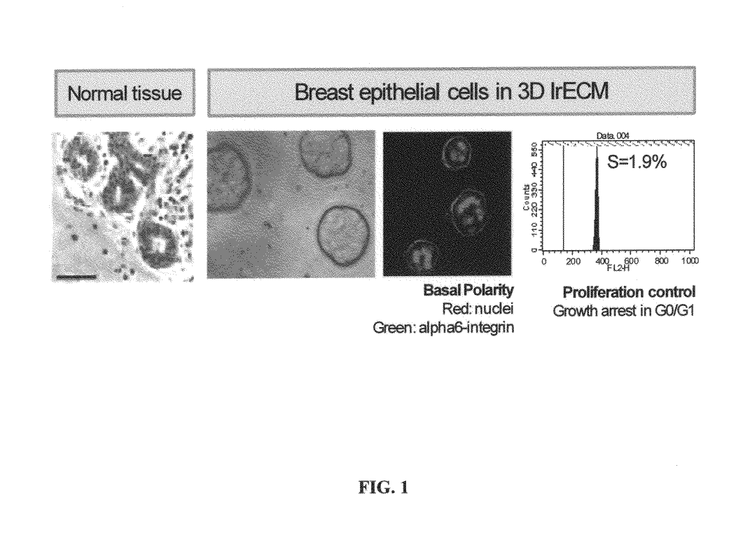

In yet another embodiment of the invention, the 3D Signature was discovered by gene expression analysis of cultured breast epithelial cells grown in a 3D model of laminin-rich extracellular matrix (1rECM). Genes down regulated during acini formation and growth arrest were identified and then tested for their ability to classify patients by long term prognosis in three unrelated sets of breast cancer patients (FIG. 1). The genes were identified and their expression levels were found to correlate with prognosis and/or response to treatment. For example, a gene signature from a tumor sample that is similar to the gene signature identified in normal cells is generally predicted to have a good prognosis and not to respond to chemotherapy, though accurate prediction requires the application of more complex equations that differ for different breast cancer subtypes.

In some embodiments, kits are provided that can include components necessary to perform such clinical tests for therapeutic sensitivity. For example, a kit may comprise one or more instruments for performing a biopsy to remove a tumor sample from a patient. In some embodiments, the kit does not comprise one or more instruments for performing a biopsy to remove a tumor sample from a patient. In some embodiments, the kit comprises an instrument for aspirating cancerous cells from tumor or cancerous growth. In some embodiments, the kit comprises components to extract genetic material (e.g. DNA, RNA, mRNA, and the like) from aspirated cells. In some embodiments, the kit comprises compositions that can be used to tag or label genetic material extracted from or derived from the aspirated cells. Genetic material that is derived from a tumor sample (e.g. aspirated cells) includes DNA or RNA that is producing using PCR, RT-PCR, RNA amplification, or any other suitable amplification method. The particular amplification method is not essential. In some embodiments, the amplification method comprises quantitative PCR. In some embodiments, the kit comprises a microarray (e.g. microarray chip) comprising hybridization probes that is specific for a genetic signature, such as but not limited to, a 3D signature generated from normal or cancerous breast epithelial cells. In some embodiments, the kit comprises a composition or product (e.g. device) that can be used to visualize the genetic material that is associated with the hybridization probes. In some embodiments, the kits are used before and after a treatment. The treatment can be of the cells ex vivo or in vivo.

In some embodiments, kits are provided for predicting response to a cancer treatment in a subject comprising one or more reagents for determining from a sample obtained from a subject expression data for at least one marker selected from the group consisting of FLJ10517, HCAP-G, CDKN3, STK6, FOXM1, FLJ10540, TNFRSF6B, HBP17, C1QDC1, TUBG1, FLJ10036, RRM2, ACTB, ACTN1, EPHA2, TRIP13, CKS2, VRK1, DUSP4, EIF4A1, SERPINE2, ODC1, or any combination thereof. The markers can be combined in any combination including, but not limited to, the other combinations described herein. In some embodiments, the kit comprises instructions for using the one or more reagents to determine expression data from the sample, wherein the instructions include instructions for determining a score from the dataset wherein the score is predictive of response to the cancer treatment. In some embodiments, the cancer treatment is a breast cancer treatment. In some embodiments, the breast cancer treatment is TFAC (a combination of taxol/fluorouracil/anthracycline/cyclophosphamide with or without filgrastim support). Chemotherapy treatments include TAC (taxol/anthracycline/cyclophosphamide with or without filgrastim support), ACMF (doxorubicin followed by cyclophosphamide, methotrexate, fluorouracil), ACT (doxorubicin, cyclophosphamide followed by taxol or docetaxel), A-T-C (doxorubicin followed by paclitaxel followed by cyclophosphamide), CAF/FAC (fluorouracil/doxorubicin/cyclophosphamide), CEF (cyclophosphamide/epirubicin/fluorouracil), AC (doxorubicin/cyclophosphamide), EC (epirubicin/cyclophosphamide), AT (doxorubicin/docetaxel or doxorubicin/taxol), CMF (cyclophosphamide/methotrexate/fluorouracil), cyclophosphamide (Cytoxan or Neosar), methotrexate, fluorouracil (5-FU), doxorubicin (Adriamycin), epirubicin (Ellence), gemcitabine, taxol (Paclitaxel), GT (gemcitabine/taxol), taxotere (Docetaxel), vinorelbine (Navelbine), capecitabine (Xeloda), platinum drugs (Cisplatin, Carboplatin), etoposide, and vinblastine. Other treatments include surgery, radiation, hormonal and targeted therapies. Additionally, other examples of cancer treatments are described elsewhere herein and a predictive score can also be determined for those.

In some embodiments, a test to determine or predict therapeutic sensitivity of a disease comprises determining the expression level of one or more markers (e.g. genes) from a patient, tissue, or cell exhibiting, or not exhibiting, symptoms of a diseased state. In some embodiments, the gene expression levels are compared to gene expression levels from a different patient known to be free of, or suspected to be free of, the disease. In some embodiments, the gene expression levels are compared to gene expression levels from a cell or tissue known to be free of, or suspected to be free of, the disease. In some embodiments, the tissue or cell known to be free of, or suspected to be free of, the disease is from the same subject (e.g. patient) who is suspected of having the disease or who is known to have the disease or known or suspected to be normal healthy tissue (either from the patient or from a healthy subject) or other diseased tissue samples and equating these expression levels with the efficacy of treatment for the diseased state. Determining the expression level for any one marker gene or set of marker genes such as those identified above and/or expression profile for any group or set of such genetic markers can be carried out by any method and may vary among embodiments of the invention. For example, in some embodiments, the expression levels of one or more markers may be measured using polymerase chain reaction (PCR), RT-PCR, enzyme-linked immunosorbent assay (ELISA), magnetic immunoassay (MIA), flow cytometry, and the like. In some embodiments, the PCR is microfluidics PCR. In other embodiments, one or more microarray may be used to measure the expression level of one or more marker genes simultaneously. Various microarray types and configurations and methods for the production of such microarrays are known in the art and are described in, for example, U.S. Patents such as: U.S. Pat. Nos. 5,445,934; 5,532,128; 5,556,752; 5,242,974; 5,384,261; 5,405,783; 5,412,087; 5,424,186; 5,429,807; 5,436,327; 5,472,672; 5,527,681; 5,529,756; 5,545,531; 5,554,501; 5,561,071; 5,571,639; 5,593,839; 5,599,695; 5,624,711; 5,658,734; and 5,700,637; the disclosures of which are hereby incorporated by reference in their entireties. Any such microarray may be useful in embodiments of the invention. For example, in some embodiments, antibodies raised against the protein product of the marker may be used as probes in microarrays of the invention such that whole cell lysate or proteins isolated from cancerous cells may be passed over the microarray and expression levels of one or more genetic marker may be reduced based on the amount of protein captured by the microarray. In other embodiments, the expression level and/or expression profile for a specific genetic marker may be carried out by extracting cellular mRNA from cancerous cells and hybridizing the mRNA directly to the array. Single-stranded antisense DNA or RNA hybridization probes specifically targeted to the mRNA marker may be used. In certain embodiments, single-stranded antisense DNA or RNA hybridization probes may be used to capture copy DNA (cDNA) or copy RNA (cRNA) that was created from mRNA extracted from cancerous cells. In some embodiments, the mRNA is amplified and/or reverse transcribed into DNA, such as cDNA. The cDNA need not be the complete coding sequence for any or all of the genes.

In some embodiments, microarray analysis may involve the measurement of an intensity of a signal received from a labeled cDNA or cRNA derived from a sample obtained from cancerous tissue that hybridizes to a known nucleic acid sequence at a specific location on a microarray. In some embodiments, the hybridization probes used in the microarrays may be nucleic acid sequences that are capable of capturing labeled cDNA or cRNA produced from the mRNA of the marker gene. In some embodiments, the intensity of the signal received and measured is proportional to the amount (e.g. quantity) of cDNA or cRNA, and thus the mRNA derived for the target gene in the cancerous tissue. Expression of the marker may occur ordinarily in a healthy subject resulting in a base steady-state level of mRNA in a healthy subject. However, in cancerous tissue, expression of the marker gene may be increased or decreased resulting in a higher level or lower level of mRNA, respectively, in diseased tissue. Alternatively, expression of a marker gene may not occur at detectable levels in normal, healthy tissue but occurs in cancerous tissue. In some embodiments, the marker is expressed at the same level in the diseased subject, tissue, or cell as compared to the healthy subject, tissue, or cell. The intensity measurements read from microarrays, as described above, may then be equated (transformed) to the degree of expression of the gene corresponding to the signal intensity of labeled cDNA or cRNA captured by the hybridization probe. Thus, the microarrays of various embodiments may detect the variability in expression by detecting differences in mRNA levels in cancerous tissue over normal tissue or standard intensities and may be used to determine a particular course of treatment for a patient whose cells or cancerous tissue is tested. The methods can be used, in some embodiments, to determine the most efficacious treatment for a patient.

In some embodiments, the method or test comprises a microarray having probes against one or more genes that exhibit a modified expression pattern or profile as a result of cancer. In some embodiments, the method or test comprises a microarray having probes against one or more genes that do not exhibit a modified expression pattern or profile as a result of cancer. The one or more genes or markers included on the array can be any one or more genes, including, for example, genes can be selected based on the likelihood that cells exhibiting the modified expression pattern or profile may be more likely to respond to a particular form of treatment. In some embodiments, the genes selected can be used to identify a cell or tumor that is less likely to respond to a particular form of treatment. For example, in some embodiments, the hybridization probes provided on the microarray may have been selected based on the ability of one or more therapeutic agents to treat tumors exhibiting an expression profile associated with such hybridization probes. Therefore, by performing the test a person can predict the efficacy of the particular form of treatment based on the gene expression pattern or profile of cells extracted from a tumor as compared to normal (e.g. non-cancerous cells).

The specific probes that are used are not essential. The probes, which can also be referred to as primers can be specific to the markers being measured and/or detected. In some embodiments, the probe comprises a sequence or a variant thereof of SEQ ID NO: @@-@@ or any combination thereof.

As used herein, “ACTB,” refers to beta-actin. In some embodiments, the beta-actin has a sequence as disclosed in GenBank Accession #NM_{—}001101 or Affymetrix Accession #200801_x_at. In some embodiments, ACTB refers to a sequence comprising SEQ ID NO: 1 or a variant thereof. In some embodiments, ACTB is detected and/or measured by a probe comprising a sequence of SEQ ID NO: 2-12 or a variant thereof or any combination thereof. In some embodiments, ACTB is detected by at least 1, 2, 3, 4, 5, 6, 7, 8, 9, or 10 probes comprising a sequence selected from the group consisting of SEQ ID NO: 2-12 or a variant thereof. In some embodiments, ACTB is detected using 11 probes, each having a different sequence and each sequence selected from the group consisting of SEQ ID NO: 2-12 or a variant thereof.

As used herein, “ACTN 1,” refers to alpha-1 actinin. In some embodiments, the alpha-1 actinin has a sequence as disclosed in GenBank Accession #NM_{—}001102 or Affymetrix Accession #208637_x_at. In some embodiments, ACTN1 refers to a sequence comprising SEQ ID NO: 13 or a variant thereof. In some embodiments, ACTN1 is detected and/or measured by a probe comprising a sequence of SEQ ID NO: 14-24 or a variant thereof or any combination thereof. In some embodiments, ACTN1 is detected by at least 1, 2, 3, 4, 5, 6, 7, 8, 9, or 10 probes comprising a sequence selected from the group consisting of SEQ ID NO: 14-24 or a variant thereof. In some embodiments, ACTN1 is detected using 11 probes, each having a different sequence and each sequence selected from the group consisting of SEQ ID NO: 14-24 or a variant thereof.

As used herein, “ASPM,”, which can also be referred to as “FLJ10517” refers to asp (abnormal spindle) homolog, microcephaly associated (Drosophila). In some embodiments, ASPM has a sequence as disclosed in GenBank Accession #NM_{—}018136 or Affymetrix Accession #219918_s_at. In some embodiments, ASPM refers to a sequence comprising SEQ ID NO: 25 or a variant thereof. In some embodiments, ASPM is detected and/or measured by a probe comprising a sequence of SEQ ID NO: 26-36 or a variant thereof or any combination thereof. In some embodiments, ASPM is detected by at least 1, 2, 3, 4, 5, 6, 7, 8, 9, or 10 probes comprising a sequence selected from the group consisting of SEQ ID NO: 26-36 or a variant thereof. In some embodiments, ASPM is detected using 11 probes, each having a different sequence and each sequence selected from the group consisting of SEQ ID NO: 26-36 or a variant thereof.

As used herein, “CEP55,”, which can also be referred to as “FLJ10540” refers to centrosomal protein 55 kDa. In some embodiments, CEP55 has a sequence as disclosed in GenBank Accession #NM_{—}001127182 or Affymetrix Accession #218542_at. In some embodiments, CEP55 refers to a sequence comprising SEQ ID NO: 37 or a variant thereof. In some embodiments, CEP55 is detected and/or measured by a probe comprising a sequence of SEQ ID NO: 38-48 or a variant thereof or any combination thereof. In some embodiments, CEP55 is detected by at least 1, 2, 3, 4, 5, 6, 7, 8, 9, or 10 probes comprising a sequence selected from the group consisting of SEQ ID NO: 38-48 or a variant thereof. In some embodiments, CEP55 is detected using 11 probes, each having a different sequence and each sequence selected from the group consisting of SEQ ID NO: 38-48 or a variant thereof.

As used herein, “CAPRIN2,”, which can also be referred to as “C1 QDC1” refers to caprin family member 2. In some embodiments, CAPRIN2 has a sequence as disclosed in GenBank Accession #NM_{—}001002259 or Affymetrix Accession #218456_at. In some embodiments, CAPRIN2 refers to a sequence comprising SEQ ID NO: 49 or a variant thereof. In some embodiments, CAPRIN2 is detected and/or measured by a probe comprising a sequence of SEQ ID NO: 50-60 or a variant thereof or any combination thereof. In some embodiments, CAPRIN2 is detected by at least 1, 2, 3, 4, 5, 6, 7, 8, 9, or 10 probes comprising a sequence selected from the group consisting of SEQ ID NO: 50-60 or a variant thereof. In some embodiments, CAPRIN2 is detected using 11 probes, each having a different sequence and each sequence selected from the group consisting of SEQ ID NO: 50-60 or a variant thereof.

As used herein, “CDKN3,” refers to cyclin-dependent kinase inhibitor 3. In some embodiments, CDKN3 has a sequence as disclosed in GenBank Accession #NM_{—}001130851 or Affymetrix Accession #209714_s_at. In some embodiments, CDKN3 refers to a sequence comprising SEQ ID NO: 61 or a variant thereof. In some embodiments, CDKN3 is detected and/or measured by a probe comprising a sequence of SEQ ID NO: 62-72 or a variant thereof or any combination thereof. In some embodiments, CDKN3 is detected by at least 1, 2, 3, 4, 5, 6, 7, 8, 9, or 10 probes comprising a sequence selected from the group consisting of SEQ ID NO: 62-72 or a variant thereof. In some embodiments, CDKN3 is detected using 11 probes, each having a different sequence and each sequence selected from the group consisting of SEQ ID NO: 62-72 or a variant thereof.

As used herein, “CKS2,” refers to CDC28 protein kinase regulatory subunit 2. In some embodiments, CKS2 has a sequence as disclosed in GenBank Accession #NM_{—}001827 or Affymetrix Accession #204170_s_at. In some embodiments, CKS2 refers to a sequence comprising SEQ ID NO: 73 or a variant thereof. In some embodiments, CKS2 is detected and/or measured by a probe comprising a sequence of SEQ ID NO: 74-84 or a variant thereof or any combination thereof. In some embodiments, CKS2 is detected by at least 1, 2, 3, 4, 5, 6, 7, 8, 9, or 10 probes comprising a sequence selected from the group consisting of SEQ ID NO: 74-84 or a variant thereof. In some embodiments, CKS2 is detected using 11 probes, each having a different sequence and each sequence selected from the group consisting of SEQ ID NO: 74-84 or a variant thereof.

As used herein, “DUSP4,” refers to dual specificity phosphatase 4. In some embodiments, DUSP4 has a sequence as disclosed in GenBank Accession #NM_{—}001394 or Affymetrix Accession #204014_at. In some embodiments, DUSP4 refers to a sequence comprising SEQ ID NO: 85 or a variant thereof. In some embodiments, DUSP4 is detected and/or measured by a probe comprising a sequence of SEQ ID NO: 86-96 or a variant thereof or any combination thereof. In some embodiments, DUSP4 is detected by at least 1, 2, 3, 4, 5, 6, 7, 8, 9, or 10 probes comprising a sequence selected from the group consisting of SEQ ID NO: 86-96 or a variant thereof. In some embodiments, DUSP4 is detected using 11 probes, each having a different sequence and each sequence selected from the group consisting of SEQ ID NO: 86-96 or a variant thereof.

As used herein, “EIF4A1,” refers to Eukaryotic translation initiation factor 4A1. In some embodiments, EIF4A1 has a sequence as disclosed in GenBank Accession #NM_{—}001416 or Affymetrix Accession #214805_at. In some embodiments, EIF4A1 refers to a sequence comprising SEQ ID NO: 97 or a variant thereof. In some embodiments, EIF4A1 is detected and/or measured by a probe comprising a sequence of SEQ ID NO: 98-108 or a variant thereof or any combination thereof. In some embodiments, EIF4A1 is detected by at least 1, 2, 3, 4, 5, 6, 7, 8, 9, or 10 probes comprising a sequence selected from the group consisting of SEQ ID NO: 98-108 or a variant thereof. In some embodiments, EIF4A1 is detected using 11 probes, each having a different sequence and each sequence selected from the group consisting of SEQ ID NO: 98-108 or a variant thereof.

As used herein, “EPHA2,” refers to EPH receptor A2. In some embodiments, EPHA2 has a sequence as disclosed in GenBank Accession #NM_{—}004431 or Affymetrix Accession #203499_at. In some embodiments, EPHA2 refers to a sequence comprising SEQ ID NO: 109 or a variant thereof. In some embodiments, EPHA2 is detected and/or measured by a probe comprising a sequence of SEQ ID NO: 110-120 or a variant thereof or any combination thereof. In some embodiments, EPHA2 is detected by at least 1, 2, 3, 4, 5, 6, 7, 8, 9, or 10 probes comprising a sequence selected from the group consisting of SEQ ID NO: 110-120 or a variant thereof. In some embodiments, EPHA2 is detected using 11 probes, each having a different sequence and each sequence selected from the group consisting of SEQ ID NO: 110-120 or a variant thereof.

As used herein, “FGFBP1,”, which can also be referred to as “HBP17” refers to fibroblast growth factor binding protein 1. In some embodiments, FGFBP 1 has a sequence as disclosed in GenBank Accession #NM_{—}005130 or Affymetrix Accession #205014_at. In some embodiments, FGFBP1 refers to a sequence comprising SEQ ID NO: 121 or a variant thereof. In some embodiments, FGFBP1 is detected and/or measured by a probe comprising a sequence of SEQ ID NO: 122-132 or a variant thereof or any combination thereof. In some embodiments, FGFBP1 is detected by at least 1, 2, 3, 4, 5, 6, 7, 8, 9, or 10 probes comprising a sequence selected from the group consisting of SEQ ID NO: 122-132 or a variant thereof. In some embodiments, FGFBP1 is detected using 11 probes, each having a different sequence and each sequence selected from the group consisting of SEQ ID NO: 122-132 or a variant thereof.

As used herein, “ZWILCH,”, which can also be referred to as “FLJ10036” refers to Zwilch, kinetochore associated, homolog (Drosophila). In some embodiments, ZWILCH has a sequence as disclosed in GenBank Accession #NM_{—}017975 or Affymetrix Accession #218349_s_at. In some embodiments, ZWILCH refers to a sequence comprising SEQ ID NO: 133 or a variant thereof. In some embodiments, ZWILCH is detected and/or measured by a probe comprising a sequence of SEQ ID NO: 134-144 or a variant thereof or any combination thereof. In some embodiments, ZWILCH is detected by at least 1, 2, 3, 4, 5, 6, 7, 8, 9, or 10 probes comprising a sequence selected from the group consisting of SEQ ID NO: 134-144 or a variant thereof. In some embodiments, ZWILCH is detected using 11 probes, each having a different sequence and each sequence selected from the group consisting of SEQ ID NO: 134-144 or a variant thereof.

As used herein, “FOXM1,” refers to forkhead box M1. In some embodiments, FOXM1 has a sequence as disclosed in GenBank Accession #NM_{—}021953 or Affymetrix Accession #202580_x_at. In some embodiments, FOXM1 refers to a sequence comprising SEQ ID NO: 145 or a variant thereof. In some embodiments, FOXM1 is detected and/or measured by a probe comprising a sequence of SEQ ID NO: 146-156 or a variant thereof or any combination thereof. In some embodiments, FOXM1 is detected by at least 1, 2, 3, 4, 5, 6, 7, 8, 9, or 10 probes comprising a sequence selected from the group consisting of SEQ ID NO: 146-156 or a variant thereof. In some embodiments, FOXM1 is detected using 11 probes, each having a different sequence and each sequence selected from the group consisting of SEQ ID NO: 146-156 or a variant thereof.

As used herein, “NCAPG,” which can also be referred to as “hCAP-G” refers to non-SMC condensin I complex, subunit G. In some embodiments, NCAPG has a sequence as disclosed in GenBank Accession #NM_{—}022346 or Affymetrix Accession #218663_at. In some embodiments, NCAPG refers to a sequence comprising SEQ ID NO: 157 or a variant thereof. In some embodiments, NCAPG is detected and/or measured by a probe comprising a sequence of SEQ ID NO: 158-168 or a variant thereof or any combination thereof. In some embodiments, NCAPG is detected by at least 1, 2, 3, 4, 5, 6, 7, 8, 9, or 10 probes comprising a sequence selected from the group consisting of SEQ ID NO: 158-168 or a variant thereof. In some embodiments, NCAPG is detected using 11 probes, each having a different sequence and each sequence selected from the group consisting of SEQ ID NO: 158-168 or a variant thereof.

As used herein, “ODC1,” refers to ornithine decarboxylase 1. In some embodiments, ODC1 has a sequence as disclosed in GenBank Accession #NM_{—}002539 or Affymetrix Accession #200790_at. In some embodiments, ODC1 refers to a sequence comprising SEQ ID NO: 169 or a variant thereof. In some embodiments, ODC1 is detected and/or measured by a probe comprising a sequence of SEQ ID NO: 170-180 or a variant thereof or any combination thereof. In some embodiments, ODC1 is detected by at least 1, 2, 3, 4, 5, 6, 7, 8, 9, or 10 probes comprising a sequence selected from the group consisting of SEQ ID NO: 170-180 or a variant thereof. In some embodiments, ODC1 is detected using 11 probes, each having a different sequence and each sequence selected from the group consisting of SEQ ID NO: 170-180 or a variant thereof.

As used herein, “RRM2,” refers to ribonucleotide reductase M2. In some embodiments, RRM2 has a sequence as disclosed in GenBank Accession #NM_{—}001034 or Affymetrix Accession #209773_s_at. In some embodiments, RRM2 refers to a sequence comprising SEQ ID NO: 181 or a variant thereof. In some embodiments, RRM2 is detected and/or measured by a probe comprising a sequence of SEQ ID NO: 182-192 or a variant thereof or any combination thereof. In some embodiments, RRM2 is detected by at least 1, 2, 3, 4, 5, 6, 7, 8, 9, or 10 probes comprising a sequence selected from the group consisting of SEQ ID NO: 182-192 or a variant thereof. In some embodiments, RRM2 is detected using 11 probes, each having a different sequence and each sequence selected from the group consisting of SEQ ID NO: 182-192 or a variant thereof.

As used herein, “SERPINE2,” serpin peptidase inhibitor, Glade E (nexin, plasminogen activator inhibitor type 1), member 2. In some embodiments, SERPINE2 has a sequence as disclosed in GenBank Accession #NM_{—}001136528 or Affymetrix Accession #212190_at. In some embodiments, SERPINE2 refers to a sequence comprising SEQ ID NO: 193 or a variant thereof. In some embodiments, SERPINE2 is detected and/or measured by a probe comprising a sequence of SEQ ID NO: 194-204 or a variant thereof or any combination thereof. In some embodiments, SERPINE2 is detected by at least 1, 2, 3, 4, 5, 6, 7, 8, 9, or 10 probes comprising a sequence selected from the group consisting of SEQ ID NO: 194-204 or a variant thereof. In some embodiments, SERPINE2 is detected using 11 probes, each having a different sequence and each sequence selected from the group consisting of SEQ ID NO: 194-204 or a variant thereof.

As used herein, “AURKA,” which can also be referred to as “STK6 refers to aurora kinase A. In some embodiments, AURKA has a sequence as disclosed in GenBank Accession #NM_{—}003600 or Affymetrix Accession #204092_s_at. In some embodiments, AURKA refers to a sequence comprising SEQ ID NO: 205 or a variant thereof. In some embodiments, AURKA is detected and/or measured by a probe comprising a sequence of SEQ ID NO: 206-216 or a variant thereof or any combination thereof. In some embodiments, AURKA is detected by at least 1, 2, 3, 4, 5, 6, 7, 8, 9, or 10 probes comprising a sequence selected from the group consisting of SEQ ID NO: 206-216 or a variant thereof. In some embodiments, AURKA is detected using 11 probes, each having a different sequence and each sequence selected from the group consisting of SEQ ID NO: 206-216 or a variant thereof.

As used herein, “RTEL1/TNFRSF6B,” refers to regulator of telomere elongation helicase 1/tumor necrosis factor receptor superfamily, member 6b, decoy. In some embodiments, RTEL1/TNFRSF6B has a sequence as disclosed in GenBank Accession #NM_{—}003823 or Affymetrix Accession #206467_x_at. In some embodiments, RTEL1/TNFRSF6B refers to a sequence comprising SEQ ID NO: 217 or a variant thereof. In some embodiments, RTEL1/TNFRSF6B is detected and/or measured by a probe comprising a sequence of SEQ ID NO: 218-228 or a variant thereof or any combination thereof. In some embodiments, RTEL1/TNFRSF6B is detected by at least 1, 2, 3, 4, 5, 6, 7, 8, 9, or 10 probes comprising a sequence selected from the group consisting of SEQ ID NO: 218-228 or a variant thereof. In some embodiments, RTEL1/TNFRSF6B is detected using 11 probes, each having a different sequence and each sequence selected from the group consisting of SEQ ID NO: 218-228 or a variant thereof.

As used herein, “TRIP13,” refers to thyroid hormone receptor interactor 13. In some embodiments, TRIP 13 has a sequence as disclosed in GenBank Accession #NM_{—}001166260 or Affymetrix Accession #204033 at. In some embodiments, TRIP13 refers to a sequence comprising SEQ ID NO: 229 or a variant thereof. In some embodiments, TRIP13 is detected and/or measured by a probe comprising a sequence of SEQ ID NO: 230-240 or a variant thereof or any combination thereof. In some embodiments, TRIP13 is detected by at least 1, 2, 3, 4, 5, 6, 7, 8, 9, or 10 probes comprising a sequence selected from the group consisting of SEQ ID NO: 230-240 or a variant thereof. In some embodiments, TRIP13 is detected using 11 probes, each having a different sequence and each sequence selected from the group consisting of SEQ ID NO: 230-240 or a variant thereof.

As used herein, “TUBG1,” refers to tubulin, gamma 1. In some embodiments, TUBG1 has a sequence as disclosed in GenBank Accession #NM_{—}001070 or Affymetrix Accession #201714_at. In some embodiments, TUBG1 refers to a sequence comprising SEQ ID NO: 241 or a variant thereof. In some embodiments, TUBG1 is detected and/or measured by a probe comprising a sequence of SEQ ID NO: 242-252 or a variant thereof or any combination thereof. In some embodiments, TUBG1 is detected by at least 1, 2, 3, 4, 5, 6, 7, 8, 9, or 10 probes comprising a sequence selected from the group consisting of SEQ ID NO: 242-252 or a variant thereof. In some embodiments, TUBG1 is detected using 11 probes, each having a different sequence and each sequence selected from the group consisting of SEQ ID NO: 242-252 or a variant thereof.

As used herein, “VRK1,” refers to vaccinia related kinase 1. In some embodiments, VRK1 has a sequence as disclosed in GenBank Accession #NM_{—}003384 or Affymetrix Accession #203856_at. In some embodiments, VRK1 refers to a sequence comprising SEQ ID NO: 253 or a variant thereof. In some embodiments, VRK1 is detected and/or measured by a probe comprising a sequence of SEQ ID NO: 254-264 or a variant thereof or any combination thereof. In some embodiments, VRK1 is detected by at least 1, 2, 3, 4, 5, 6, 7, 8, 9, or 10 probes comprising a sequence selected from the group consisting of SEQ ID NO: 254-264 or a variant thereof. In some embodiments, VRK1 is detected using 11 probes, each having a different sequence and each sequence selected from the group consisting of SEQ ID NO: 254-264 or a variant thereof.

The sequences referred to in the section above are described in the sequence listing and in the following table. The sequences can also be the reverse (3′-5′) orientation or a variant thereof.

| | Affymetrix | GenBank | |
|---|---|---|---|
| Gene | accession | Accession | |
| symbol | number | No. | Probe Sequences |
| ACTB | 200801_x_at | NM_001101 | TATGACTTAGTTGCGTTACACCCTT (SEQ ID NO: 2) |
| | | | CAGCAGTCGGTTGGAGCGAGCATCC (SEQ ID NO: 3) |
| | | | GCATCCCCCAAAGTTCACAATGTGG (SEQ ID NO: 4) |
| | | | GGCCGAGGACTTTGATTGCACATTG (SEQ ID NO: 5) |
| | | | TTGTTACAGGAAGTCCCTTGCCATC (SEQ ID NO: 6) |
| | | | TAAGGAGAATGGCCCAGTCCTCTCC (SEQ ID NO: 7) |
| | | | TTTTGAATGATGAGCCTTCGTGCCC (SEQ ID NO: 8) |
| | | | TTTTTGTCCCCCAACTTGAGATGTA (SEQ ID NO: 9) |
| | | | TGTATGAAGGCTTTTGGTCTCCCTG (SEQ ID NO: 10) |
| | | | GGAGTGGGTGGAGGCAGCCAGGGCT (SEQ ID NO: 11) |
| | | | GCCAGGGCTTACCTGTACACTGACT (SEQ ID NO: 12) |
| ACTN1 | 208637_x_at | NM_001102 | GGTCCCGAGGAGTTCAAAGCCTGCC (SEQ ID NO: 14) |
| | | | GCAGAATTTGCCCGCATCATGAGCA (SEQ ID NO: 15) |
| | | | TCATGAGCATTGTGGACCCCAACCG (SEQ ID NO: 16) |
| | | | TGGGGGTAGTGACATTCCAGGCCTT (SEQ ID NO: 17) |
| | | | AGCAGACCAAGTCATGGCTTCCTTC (SEQ ID NO: 18) |
| | | | CTTCCTTCAAGATCCTGGCTGGGGA (SEQ ID NO: 19) |
| | | | TACATTACCATGGACGAGCTGCGCC (SEQ ID NO: 20) |
| | | | CCGACCAGGCTGAGTACTGCATCGC (SEQ ID NO: 21) |
| | | | AGGTGCTCTGGACTACATGTCCTTC (SEQ ID NO: 22) |
| | | | GGCGCTGTACGGCGAGAGTGACCTC (SEQ ID NO: 23) |
| | | | CCCTGCCCGCGAAGTGACAGTTTAC (SEQ ID NO: 24) |
| ASPM | 219918_s_at | NM_018136 | GTTGTAATCGCAGTATTCCTTGTAT (SEQ ID NO: 26) |
| | | | TCAGATATGCTGTGCAAGTCTTGCT (SEQ ID NO: 27) |
| | | | GGAGCTTTTGCAGATATACCGAGAA (SEQ ID NO: 28) |
| | | | GTTGTTTGTTGGCTATTTTACTGAA (SEQ ID NO: 29) |
| | | | ATAGAGCCTCTGATGTACGAAGTAG (SEQ ID NO: 30) |
| | | | GTTGTTGACCGTATTTACAGTCTCT (SEQ ID NO: 31) |
| | | | CAGTCTCTACAAACTTACAGCTCAT (SEQ ID NO: 32) |
| | | | GCATTCCTTTTATCCCAGAAACACC (SEQ ID NO: 33) |
| | | | GAAGAAATCACAAATCCCCTGCAAG (SEQ ID NO: 34) |
| | | | AATCCCCTGCAAGCTATTCAAATGG (SEQ ID NO: 35) |
| | | | GTGATGGATACGCTTGGCATTCCTT (SEQ ID NO: 36) |
| CEP55 | 218542_at | NM_001127182 | AAGGATCTTAACTGTGTTCGCATTT (SEQ ID NO: 38) |
| | | | GTTCGCATTTTTTATCCAAGCACTT (SEQ ID NO: 39) |
| | | | AATCCTAATTTTGATGTCCATTGTT (SEQ ID NO: 40) |
| | | | GTTGGGGATTTTCTTGATCTTTATT (SEQ ID NO: 41) |
| | | | TATTGCTGCTTACCATTGAAACTTA (SEQ ID NO: 42) |
| | | | TGAAACTTAACCCAGCTGTGTTCCC (SEQ ID NO: 43) |
| | | | AACTCTGTTCTGCGCACGAAACAGT (SEQ ID NO: 44) |
| | | | TTAAGTGGCCACACACAATGTTTTC (SEQ ID NO: 45) |
| | | | GTTTTCTCTTATGTTATCTGGCAGT (SEQ ID NO: 46) |
| | | | GCCCTCTCATTTGATTGACAGTATT (SEQ ID NO: 47) |
| | | | AGGTTTTCTAACATGCTTACCACTG (SEQ ID NO: 48) |
| CAPRIN2 | 218456_at | NM_001002259 | GAATGTGCCACTGTATGTCAACCTC (SEQ ID NO: 50) |
| | | | AGAGGTCTTGGTATCAGCCTATGCC (SEQ ID NO: 51) |
| | | | GCCTATGCCAATGATGGTGCTCCAG (SEQ ID NO: 52) |
| | | | GGTGCTCCAGACCATGAAACTGCTA (SEQ ID NO: 53) |
| | | | GCAATCATGCAATTCTTCAGCTCTT (SEQ ID NO: 54) |
| | | | GATATGGTTACGTCTGCACAGGGGA (SEQ ID NO: 55) |
| | | | ATATTCTACGTTTTCAGGCTATCTT (SEQ ID NO: 56) |
| | | | TCTTTGCCCTCATGACTGATTGGTT (SEQ ID NO: 57) |
| | | | GTAGCCTCGCTAGTCAAGCTGTGAA (SEQ ID NO: 58) |
| | | | AGCTTACTAAACTGACTGCCTCAAG (SEQ ID NO: 59) |
| | | | GTTACAATGCCTTGTTGTGCCTCAA (SEQ ID NO: 60) |
| CDKN3 | 209714_s_at | NM_001130851 | TTTCTCGGTTTATGTGCTCTTCCAG (SEQ ID NO: 62) |
| | | | TAGAGTCCCAAACCTTCTGGATCTC (SEQ ID NO: 63) |
| | | | GGATCTCTACCAGCAATGTGGAATT (SEQ ID NO: 64) |
| | | | ACCCATCATCATCCAATCGCAGATG (SEQ ID NO: 65) |
| | | | CTCCTGACATAGCCAGCTGCTGTGA (SEQ ID NO: 66) |
| | | | TGGAAGAGCTTACAACCTGCCTTAA (SEQ ID NO: 67) |
| | | | GGAGGACTTGGGAGATCTTGTCTTG (SEQ ID NO: 68) |
| | | | GACACAATATCACCAGAGCAAGCCA (SEQ ID NO: 69) |
| | | | AAGCCATAGACAGCCTGCGAGACCT (SEQ ID NO: 70) |
| | | | GAGGATCCGGGGCAATACAGACCAT (SEQ ID NO: 71) |
| | | | ATTAGCTGCACATCTATCATCAAGA (SEQ ID NO: 72) |
| CKS2 | 204170_s_at | NM_001827 | CGCTCTCGTTTCATTTTCTGCAGCG (SEQ ID NO: 74) |
| | | | CGACGAACACTACGAGTACCGGCAT (SEQ ID NO: 75) |
| | | | TTATGTTACCCAGAGAACTTTCCAA (SEQ ID NO: 76) |
| | | | ACTTGGTGTCCAACAGAGTCTAGGC (SEQ ID NO: 77) |
| | | | TATTCTTCTCTTTAGACGACCTCTT (SEQ ID NO: 78) |
| | | | TCTCTTTAGACGACCTCTTCCAAAA (SEQ ID NO: 79) |
| | | | ACAAATCTTTCATCCATACCTGTGC (SEQ ID NO: 80) |
| | | | GTGCATGAGCTGTATTCTTCACAGC (SEQ ID NO: 81) |
| | | | GCAACAGAGCTCAGTTAAATGCAAC (SEQ ID NO: 82) |
| | | | GATAAAAGTTCTTCCAGTCAGTTTT (SEQ ID NO: 83) |
| | | | CAGTCAGTTTTTCTCTTAAGTGCCT (SEQ ID NO: 84) |
| DUSP4 | 204014_at | NM_001394 | GAAGGTGTGGTTTTCATTTCTCAGT (SEQ ID NO: 86) |
| | | | ATTTCTCAGTCACCAACAGATGAAT (SEQ ID NO: 87) |
| | | | ATGTCAAACAGCTGAGCACCGTAGC (SEQ ID NO: 88) |
| | | | GAGCACCGTAGCATGCAGATGTCAA (SEQ ID NO: 89) |
| | | | GCAGATGTCAAGGCAGTTAGGAAGT (SEQ ID NO: 90) |
| | | | AATGGTGTCTTGTAGATATGTGCAA (SEQ ID NO: 91) |
| | | | TGCAAGGTAGCATGATGAGCAACTT (SEQ ID NO: 92) |
| | | | GAGCAACTTGAGTTTGTTGCCACTG (SEQ ID NO: 93) |
| | | | GCCACTGAGAAGCAGGCGGGTTGGG (SEQ ID NO: 94) |
| | | | TATGTTGCCAAGGCTCATCTTGAGA (SEQ ID NO: 95) |
| | | | TTGAGAAGCAGGCGGGTTGGGTGGG (SEQ ID NO: 96) |
| EIF4A1 | 214805_at | NM_001416 | CCTTTTCACCCTTGCTTAATAGCCA (SEQ ID NO: 98) |
| | | | TTAATAGCCAGAGCTGTTTCATGCC (SEQ ID NO: 99) |
| | | | CACACAATTCTAATGCTGGACTTTT (SEQ ID NO: 100) |
| | | | CTTTTTCCTGGGTCATGCTGCAACA (SEQ ID NO: 101) |
| | | | GCAGAGCTCCATTCTAAGGCACTTG (SEQ ID NO: 102) |
| | | | TTCTAAGGCACTTGGCTCTCAGTTT (SEQ ID NO: 103) |
| | | | GGCTCTCAGTTTTCTCAGAGTGAAC (SEQ ID NO: 104) |
| | | | AGTGAACATGCCTCGTAGCTTGGGT (SEQ ID NO: 105) |
| | | | TCGTAGCTTGGGTCCTATGGCAGGA (SEQ ID NO: 106) |
| | | | TGCATCACCTGTTCTATAAAACTGG (SEQ ID NO: 107) |
| | | | GGCTCAACTCGTATAATCCCAACAC (SEQ ID NO: 108) |
| EPHA2 | 203499_at | NM_004431 | TATAGGATATTCCCAAGCCGACCTT (SEQ ID NO: 110) |
| | | | TGGCCCAGCGCCAAGTAAACAGGGT (SEQ ID NO: 111) |
| | | | TAAACAGGGTACCTCAAGCCCCATT (SEQ ID NO: 112) |
| | | | GGGCAGACTGTGAACTTGACTGGGT (SEQ ID NO: 113) |
| | | | CTGGGTGAGACCCAAAGCGGTCCCT (SEQ ID NO: 114) |
| | | | TCCTGGGCCTTTGCAAGATGCTTGG (SEQ ID NO: 115) |
| | | | AGATGCTTGGTTGTGTTGAGGTTTT (SEQ ID NO: 116) |
| | | | GGGTGTCAAACATTCGTGAGCTGGG (SEQ ID NO: 117) |
| | | | AGGGACCGGTGCTGCAGGAGTGTCC (SEQ ID NO: 118) |
| | | | CCCATCTCTCATCCTTTTGGATAAG (SEQ ID NO: 119) |
| | | | GATAAGTTTCTATTCTGTCAGTGTT (SEQ ID NO: 120) |
| FGFBP1 | 205014_at | NM_005130 | AACAGAGATGTCCCCCAGGGAGCAC (SEQ ID NO: 122) |
| | | | GCCACCAAAGCTCCCGAGTGTGTGG (SEQ ID NO: 123) |
| | | | CAGAGGAAGACTGCCCTGGAGTTCT (SEQ ID NO: 124) |
| | | | ACATTCTTCCTCAGCATAGTGCAGG (SEQ ID NO: 125) |
| | | | AGTGCAGGACACGTCATGCTAATGA (SEQ ID NO: 126) |
| | | | GAGATGTCATGTCGTAAGTCCCTCT (SEQ ID NO: 127) |
| | | | TACTTTAAAGCTCTCTACAGTCCCC (SEQ ID NO: 128) |
| | | | TCTACAGTCCCCCCAAAATATGAAC (SEQ ID NO: 129) |
| | | | GAGGCTGTTTCCTGCAGCATGTATT (SEQ ID NO: 130) |
| | | | TCCATGGCCCACACAGCTATGTGTT (SEQ ID NO: 131) |
| | | | TTTCAGTGCAACGAACTTTCTGCTG (SEQ ID NO: 132) |
| ZWILCH | 218349_s_at | NM_017975 | GGAACCATGGACACAGTTTCTCTCA (SEQ ID NO: 134) |
| | | | CAGTTTCTCTCAGTGGGACTATTCC (SEQ ID NO: 135) |
| | | | CATAGGTCAGGAACTTGCATCTTTG (SEQ ID NO: 136) |
| | | | GAATACTTCATTGCTCCATCAGTAG (SEQ ID NO: 137) |
| | | | TATCGTGTCCAAAAACTCCACCATA (SEQ ID NO: 138) |
| | | | AATATTAGTCAGTTGCATGCCTTTC (SEQ ID NO: 139) |
| | | | GCATGCCTTTCATTAAATCTCAACA (SEQ ID NO: 140) |
| | | | ATCTCAACATGAACTCCTCTTTTCT (SEQ ID NO: 141) |
| | | | CTGCCAGTCAGACCAACTGCTGTAA (SEQ ID NO: 142) |
| | | | TTACTAACATGGTTACCTGCAGCCA (SEQ ID NO: 143) |
| | | | GCAGCCAGGTGCATTTCAAGTGAAG (SEQ ID NO: 144) |
| FOXM1 | 202580_x_at | NM_021953 | TCAATTGACTTCTGTTCCTTGCTTT (SEQ ID NO: 146) |
| | | | AAGACCTGCAGTGCACGGTTTCTTC (SEQ ID NO: 147) |
| | | | CGGTTTCTTCCAGGCTGAGGTACCT (SEQ ID NO: 148) |
| | | | GAGGTACCTGGATCTTGGGTTCTTC (SEQ ID NO: 149) |
| | | | TGGGTTCTTCACTGCAGGGACCCAG (SEQ ID NO: 150) |
| | | | AAGTGGATCTGCTTGCCAGAGTCCT (SEQ ID NO: 151) |
| | | | TGTTTCCAAGTCAGCTTTCCTGCAA (SEQ ID NO: 152) |
| | | | GTGCCCAGATGTGCGCTATTAGATG (SEQ ID NO: 153) |
| | | | GATGTTTCTCTGATAATGTCCCCAA (SEQ ID NO: 154) |
| | | | TTGCCCCTCAGCTTTGCAAAGAGCC (SEQ ID NO: 155) |
| | | | CCAGCTGACCGCATGGGTGTGAGCC (SEQ ID NO: 156) |
| NCAPG | 218663_at | NM_022346 | AATTCGAGTCTATACAAAAGCCTTG (SEQ ID NO: 158) |
| | | | AGTTCTTTAGAACTCAGTAGCCATC (SEQ ID NO: 159) |
| | | | GTAGCCATCTTGCAAAAGATCTTCT (SEQ ID NO: 160) |
| | | | AAGATCTTCTGGTTCTATTGAATGA (SEQ ID NO: 161) |
| | | | AGGACATGTCTGAGAGCTTTGGAGA (SEQ ID NO: 162) |
| | | | ATTTGGTGACCAAGCTGAAGCAGCA (SEQ ID NO: 163) |
| | | | TGAAGCAGCACAGGATGCCACCTTG (SEQ ID NO: 164) |
| | | | GAAGTATATATGACTCCACTCAGGG (SEQ ID NO: 165) |
| | | | GACTCCACTCAGGGGTGTAAAAGCA (SEQ ID NO: 166) |
| | | | CCAAGCATCAAAGTCTACTCAGCTA (SEQ ID NO: 167) |
| | | | GTGACAGTTTCAGCTAGGACGAACA (SEQ ID NO: 168) |
| ODC1 | 200790_at | NM_002539 | AAAACATGGGCGCTTACACTGTTGC (SEQ ID NO: 170) |
| | | | TGCTGCCTCTACGTTCAATGGCTTC (SEQ ID NO: 171) |
| | | | CCAGAGGCCGACGATCTACTATGTG (SEQ ID NO: 172) |
| | | | TACTATGTGATGTCAGGGCCTGCGT (SEQ ID NO: 173) |
| | | | GCCTGCGTGGCAACTCATGCAGCAA (SEQ ID NO: 174) |
| | | | GCAGCCTGTGCTTCGGCTAGTATTA (SEQ ID NO: 175) |
| | | | AGCACTCTGGTAGCTGTTAACTGCA (SEQ ID NO: 176) |
| | | | AGAGTAGGGTCGCCATGATGCAGCC (SEQ ID NO: 177) |
| | | | GGGTCACACTTATCTGTGTTCCTAT (SEQ ID NO: 178) |
| | | | TTATTCACTCTTCAGACACGCTACT (SEQ ID NO: 179) |
| | | | AGACACGCTACTCAAGAGTGCCCCT (SEQ ID NO: 180) |
| RRM2 | 209773_s_at | NM_001034 | TTTTACCTTGGATGCTGACTTCTAA (SEQ ID NO: 182) |
| | | | GAAGATGTGCCCTTACTTGGCTGAT (SEQ ID NO: 183) |
| | | | GAAGTGTTACCAACTAGCCACACCA (SEQ ID NO: 184) |
| | | | CTAGCCACACCATGAATTGTCCGTA (SEQ ID NO: 185) |
| | | | AACTGTGTAGCTACCTCACAACCAG (SEQ ID NO: 186) |
| | | | CTCACAACCAGTCCTGTCTGTTTAT (SEQ ID NO: 187) |
| | | | GTGCTGGTAGTATCACCTTTTGCCA (SEQ ID NO: 188) |
| | | | CCTGGCTGGCTGTGACTTACCATAG (SEQ ID NO: 189) |
| | | | GACCCTTTAGTGAGCTTAGCACAGC (SEQ ID NO: 190) |
| | | | TAAACAGTCCTTTAACCAGCACAGC (SEQ ID NO: 191) |
| | | | CAGCCTCACTGCTTCAACGCAGATT (SEQ ID NO: 192) |
| SERPINE2 | 212190_at | NM_001136528 | CGATGCAAGTGTTTCTGTTCTGGGA (SEQ ID NO: 194) |
| | | | GGATGGCTGGAACACTGTACTGAGG (SEQ ID NO: 195) |
| | | | TAAACTACTGAACTGTTACCTAGGT (SEQ ID NO: 196) |
| | | | AACAACCCTGTTGAGTATTTGCTGT (SEQ ID NO: 197) |
| | | | GAGTATTTGCTGTTTGTCCAGTTCA (SEQ ID NO: 198) |
| | | | GTTTTGTCTATATGTGCGGCTTTTC (SEQ ID NO: 199) |
| | | | TCCCCCTCCAAAGTCTTGATAGCAA (SEQ ID NO: 200) |
| | | | AAACGGTGAAATCTCTAGCCTCTTT (SEQ ID NO: 201) |
| | | | TTAAAAAACTCCTGTCTTGCTAGAC (SEQ ID NO: 202) |
| | | | TGTTGTGCAGTGTGCCTGTCACTAC (SEQ ID NO: 203) |
| | | | ACTGGTCTGTACTCCTTGGATTTGC (SEQ ID NO: 204) |
| AURKA | 204092_s_at | NM_003600 | TGCCCTGACCCCGATCAGTTAAGGA (SEQ ID NO: 206) |
| | | | GACCCCGATCAGTTAAGGAGCTGTG (SEQ ID NO: 207) |
| | | | GAGCTGTGCAATAACCTTCCTAGTA (SEQ ID NO: 208) |
| | | | GCTGTGCAATAACCTTCCTAGTACC (SEQ ID NO: 209) |
| | | | AAAGCTGTTGGAATGAGTATGTGAT (SEQ ID NO: 210) |
| | | | TTGTATTTTTTCTCTGGTGGCATTC (SEQ ID NO: 211) |
| | | | TTTTTTCTCTGGTGGCATTCCTTTA (SEQ ID NO: 212) |
| | | | TTCTCTGGTGGCATTCCTTTAGGAA (SEQ ID NO: 213) |
| | | | ATTCCTTTAGGAATGCTGTGTGTCT (SEQ ID NO: 214) |
| | | | TTAACCACTTATCTCCCATATGAGA (SEQ ID NO: 215) |
| | | | CACTTATCTCCCATATGAGAGTGTG (SEQ ID NO: 216) |
| RTEL1/ | 206467_x_at | NM_003823 | GCAGCTCCAGCTCAGAGCAGTGCCA (SEQ ID NO: 218) |
| TNFRSF6B | | | GGGCCTGGCCCTCAATGTGCCAGGC (SEQ ID NO: 219) |
| | | | AGCACCAGGGTACCAGGAGCTGAGG (SEQ ID NO: 220) |
| | | | AGCTGAGGAGTGTGAGCGTGCCGTC (SEQ ID NO: 221) |
| | | | TGCCGTCATCGACTTTGTGGCTTTC (SEQ ID NO: 222) |
| | | | TTTGTGGCTTTCCAGGACATCTCCA (SEQ ID NO: 223) |
| | | | GACATCTCCATCAAGAGGCTGCAGC (SEQ ID NO: 224) |
| | | | GAGGCTGCAGCGGCTGCTGCAGGCC (SEQ ID NO: 225) |
| | | | TGCAGCTGAAGCTGCGTCGGCGGCT (SEQ ID NO: 226) |
| | | | CCCTCTTATTTATTCTACATCCTTG (SEQ ID NO: 227) |
| | | | GCACCCCACTTGCACTGAAAGAGGC (SEQ ID NO: 228) |
| TRIP13 | 204033_at | NM_001166260 | GAAGAACCATCGAAACCTGTTTGTT (SEQ ID NO: 230) |
| | | | AAATGCACACATTACTCCAGGTGGA (SEQ ID NO: 231) |
| | | | GGTGGCAATTGCTTTCTGATATCAG (SEQ ID NO: 232) |
| | | | ATCAAGACATGGTCCCATTTGCAGG (SEQ ID NO: 233) |
| | | | GTGCAGACTCTGAGTGTTCCAGGGA (SEQ ID NO: 234) |
| | | | GAAACACATGCTGGACATCCCTTGT (SEQ ID NO: 235) |
| | | | CATCCCTTGTAACCCGGTATGGGCG (SEQ ID NO: 236) |
| | | | CTGCATTGCTGGGATGTTTCTGCCC (SEQ ID NO: 237) |
| | | | CTGCCCACGGTTTTGTTTGTGCAAT (SEQ ID NO: 238) |
| | | | ATAGGTCAGTTACTGGTCTCTTTCT (SEQ ID NO: 239) |
| | | | GGTCTCTTTCTGCCGAATGTTATGT (SEQ ID NO: 240) |
| TUBG1 | 201714_at | NM_001070 | CTCTTCGAGAGAACCTGTCGCCAGT (SEQ ID NO: 242) |
| | | | CGAGAGAACCTGTCGCCAGTATGAC (SEQ ID NO: 243) |
| | | | GTCGCCAGTATGACAAGCTGCGTAA (SEQ ID NO: 244) |
| | | | GCCAGTATGACAAGCTGCGTAAGCG (SEQ ID NO: 245) |
| | | | CCTTCCTGGAGCAGTTCCGCAAGGA (SEQ ID NO: 246) |
| | | | GACACATCCAGGGAGATTGTGCAGC (SEQ ID NO: 247) |
| | | | GCAGCTCATCGATGAGTACCATGCG (SEQ ID NO: 248) |
| | | | ACCCCCTCAGAGCACAGATCAGGGA (SEQ ID NO: 249) |
| | | | CCTCAGAGCACAGATCAGGGACCTC (SEQ ID NO: 250) |
| | | | TCTCTTTCTCATATACATGGACTCT (SEQ ID NO: 251) |
| | | | CATATACATGGACTCTCTGTTGGCC (SEQ ID NO: 252) |
| VRK1 | 203856_at | NM_003384 | AAATTGGACCTCAGTGTTGTGGAGA (SEQ ID NO: 254) |
| | | | GAACCTGGTGTTGAAGATACGGAAT (SEQ ID NO: 255) |
| | | | GATACGGAATGGTCAAACACACAGA (SEQ ID NO: 256) |
| | | | ACAGACAGAGGAGGCCATACAGACC (SEQ ID NO: 257) |
| | | | CCATACAGACCCGTTCAAGAACCAG (SEQ ID NO: 258) |
| | | | TCAGATGCTGTGAACCAGATTTCCT (SEQ ID NO: 259) |
| | | | GTGAGTCTTGCGAGGTGGAATTAAT (SEQ ID NO: 260) |
| | | | TACTCCTTAAGTTATCCCAAAGCCG (SEQ ID NO: 261) |
| | | | ATCCCAAAGCCGTGTGTTTGTGATG (SEQ ID NO: 262) |
| | | | GACACGCACTTTTCTAATCATTGTA (SEQ ID NO: 263) |
| | | | AAATGTTTGACAAAGTCCTCACTTT (SEQ ID NO: 264) |

Embodiments are not limited based on the number of genes or the specific genes whose expression may be assessed or the type of treatment or therapeutic whose efficacy can be tested using the clinical test. For example, in some embodiments, the microarray may include probes for from 1 to greater than 500 genes whose expression patterns are modified in tumors or cancerous cells. In other embodiments, the microarray may include hybridization probes for from 2 to about 300, from about 5 to about 100, from about 10 to about 50, or from about 10 to about 25 genes. Without wishing to be bound by theory, microarrays including a larger number of hybridization probes such as, for example, 100 or more, 200 or more, 300 or more, or 500 or more may be capable to test for the efficacy of a greater number of therapeutic agents in a single test, whereas a microarray including a limited number of hybridization probes such as, for example, up to 5, up to 10, up to 15, up to 20, up to 25, up to 30, or up to 50, may be capable of more definitively testing the efficacy of a particular form of treatment. In some embodiments, the microarray may include probes for from 15 to 30 genes such as 15, 16, 17, 18, 19, 20, 21, 22, 23, 24, 25, 26, 27, 28, 29, or 30 probes.

Similarly, the microarray may be prepared to test the expression level of any known gene or any gene that may be discovered that exhibits a change in expression in tumorigenic cells as compared to normal cells and which change in expression may be indicative of cells that respond to a specific form of treatment. In some embodiments, non-limiting examples of genes associated with various types of cancer, i.e., “genetic markers” or “marker genes”, whose expression can be tested using the tests and microarrays may include, but are not limited to, AC004010, ACTB, ACTN1, APOE, ASPM, AURKA, BBOX1, BIRC5, BLM, BM039, BNIP3L, C1QDC1, C140RF147, CDC6, CDC45L, CDK3, CDKN3, CENPA, CEP55, CKS2, COL4A2, CRYAB, DC13, DSG3, DUSP4, EFEMP1, EGR1, EIF4A1, EIF4B, EPHA2, EPHA2, FEN1, FGFBP1, FKBP1B, FLJ10036, FLJ10517, FLJ10540, FLJ10687, FLJ20701, FOSL2, FOXM1, GPNMB, H2AFZ, HCAP-G, HBP17, HPV17, ID-GAP, IGFBP2, KIAA084, KIAA092, KNSL6, KNTC2, KRTC2, KRT10, LEPL, L0051203, L0051659, LRP16, LRP8, MAFB, MCM6, MELK, MTB, NCAPG, NUSAP1, ODC, ODC1, PHLDA1, PITRM1, PLK1, POLQ, PPL, PRC1, RAMP, RRM2, RRM3, SEC4L, SEPT10, SERPINE2, SERPINA3, SLC20A1, SMC4L1, SNRPA1, SOX4, SRCAP, SRD5A1, STK6, SUCLG2, SUPT16H, TCF4, THBS1, TNFRSF6B, TRIP13, TUBG1, UCHL5, VRK1, WDR32, ZNF227, ZWILICH, and the like and combinations thereof. In some embodiments, the marker genes whose expression levels can be tested, measured, quantified, or determined are FLJ10517, HCAP-G, CDKN3, STK6, FOXM1, FLJ10540, TNFRSF6B, HBP17, C1QDC1, TUBG1, FLJ10036, RRM2, ACTB, ACTN1, EPHA2, TRIP13, CKS2, VRK1, DUSP4, EIF4A1, SERPINE2, ODC1, and the like and any combinations thereof. For example any marker can be combined with any other marker or any other multiple markers. The hybridization probes selected for the microarray may include any number and type of marker genes necessary to assure accurate and precise results, and in some embodiments, the number of hybridization probes may be economized to include, for example, a subset of genes whose expression profile is indicative of a particular type of cancer and/or treatment for which the microarray is designed to test.

Numerous techniques and methods are available for detecting intensity changes and making intensity measurements from microarrays to determine levels gene expression including, for example, the methods found in U.S. Pat. Nos. 6,271,002; 6,218,122; 6,218,114; and 6,004,755, the disclosure of each of which are hereby incorporated by reference in their entireties. In some embodiments, expression levels of one or more genetic markers may be conducted by comparing the intensity measurements derived from the microarrays. For example, in some embodiments, intensity measurement comparisons may be used to generate a ratio matrix of the expression intensities of genes in a test sample taken from cancerous tissue versus those in a control sample from normal tissue of the same type or of a previously collected sample of diseased tissue. The ratio of these expression intensities may indicate a change in gene expression between the test and control samples and may be used to determine, for example, the progression of the cancer, the likelihood that a particular form of therapy will be effective, and/or the effect a particular form of treatment has had on the patient.

In various embodiments, modulated genes may be defined as those genes that are differentially expressed in cancerous tissue as being either up regulated or down regulated. Up regulation and down regulation are relative terms meaning that a detectable difference, beyond the contribution of noise in the system used to measure it, may be found in the amount of expression of genes relative to some baseline. In some embodiments, a baseline expression level may be measured from the amount of mRNA for a particular genetic marker in a normal cell or other standard cell (i.e. positive or negative control). The one or more genetic markers in the cancerous tissue may be either up regulated or down regulated relative to the baseline level using the same measurement method. Distinctions between expression of a genetic marker in healthy tissue versus cancerous tissue may be made through the use of mathematical/statistical values that are related to each other. For example, in some embodiments, distinctions may be derived from a mean signal indicative of gene expression in normal, healthy tissue and variation from this mean signal may be interpreted as being indicative of cancerous tissue. In other embodiments, distinctions may be made by use of the mean signal ratios between different groups of readings, i.e. intensity measurements, and the standard deviations of the signal ratio measurements. A great number of such mathematical/statistical values can be used in their place such as return at a given percentile. Regardless of the purpose, the expression of one or more markers can be determined using a microarray. These values can then be used to determine whether a cancer or tumor will likely respond to a treatment. The expression levels can be also be determined by using PCR, RT-PCR, RNA amplification, or any other method suitable for determining expression levels of one or more markers. A standard can be used in conjunction with the one or more markers to determine the expression level of the one or more markers. The expression levels are then used in an equation or algorithm and the expression levels are transformed into a predictive number. The predictive number can indicate that the tumor or cancer will likely respond to treatment or that the cancer or tumor will not likely respond to treatment.

By determining the expression levels of genes that exhibit modulated expression in diseased, or cancerous tissue, a expression profile or genetic signature for particular diseased states may be determined. Accordingly, in some embodiments, the expression profile for various disease types and various patients may vary, patients who are more likely to respond to specific types of therapy can be identified. For example, in some embodiments, the tests may include a microarray configured to identify patients who will respond to a specific form of therapy based on their particular genetic profile, such as, but not limited to, the 3-D signature. For example, in some embodiments, the microarray may include a set of genes specifically associated with the diseased state. For example, in some embodiments, the microarray of the test may comprise a set of 10-30 markers (e.g. genes) associated with cancer, and in some embodiments, the cancer tested using a test may be breast cancer.

In some embodiments, a test for breast cancer comprises a microarray may comprise probes for FLJ10517, HCAP-G, CDKN3, STK6, FOXM1, FLJ10540, TNFRSF6B, HBP17, C1QDC1, TUBG1, FLJ10036, RRM2, ACTB, ACTN1, EPHA2, TRIP13, CKS2, VRK1, DUSP4, EIF4A1, SERPINE2, or ODC1, and any combination thereof. In some embodiments, the microarray comprises FLJ10517, HCAP-G, CDKN3, STK6, FOXM1, FLJ10540, TNFRSF6B, HBP17, C1QDC1, TUBG1, FLJ10036, RRM2, ACTB, ACTN1, EPHA2, TRIP13, CKS2, VRK1, DUSP4, EIF4A1, SERPINE2, and ODC1. In some embodiments, the microarray comprises FLJ10517 and HCAP-G. In some embodiments, the microarray comprises FLJ10517, HCAP-G, and CDKN3. In some embodiments, the microarray comprises FLJ10517, HCAP-G, CDKN3, and STK6. In some embodiments, the microarray comprises FLJ10517, HCAP-G, CDKN3, STK6, and FOXM1. In some embodiments, the microarray comprises FLJ10517, HCAP-G, CDKN3, STK6, FOXM1, and FLJ10540. In some embodiments, the microarray comprises FLJ10517, HCAP-G, CDKN3, STK6, FOXM1, FLJ1110540, and TNFRSF6B. In some embodiments, the microarray comprises FLJ1110517, HCAP-G, CDKN3, STK6, FOXM1, FLJ10540, TNFRSF6B, and HBP17. In some embodiments, the microarray comprises FLJ10517, HCAP-G, CDKN3, STK6, FOXM1, FLJ10540, TNFRSF6B, HBP17, and C1QDC1. In some embodiments, the microarray comprises FLJ1110517, HCAP-G, CDKN3, STK6, FOXM1, FLJ10540, TNFRSF6B, HBP17, C1QDC1, and TUBG1. In some embodiments, the microarray comprises FLJ10517, HCAP-G, CDKN3, STK6, FOXM1, FLJ10540, TNFRSF6B, HBP17, C1QDC1, TUBG1, and FLJ10036. In some embodiments, the microarray comprises FLJ10517, HCAP-G, CDKN3, STK6, FOXM1, FLJ10540, TNFRSF6B, HBP17, C1QDC1, TUBG1, FLJ10036, and RRM2. In some embodiments, the microarray comprises FLJ1110517, HCAP-G, CDKN3, STK6, FOXM1, FLJ10540, TNFRSF6B, HBP17, C1QDC1, TUBG1, FLJ10036, RRM2, and ACTB. In some embodiments, the microarray comprises FLJ10517, HCAP-G, CDKN3, STK6, FOXM1, FLJ10540, TNFRSF6B, HBP17, C1QDC1, TUBG1, FLJ10036, RRM2, ACTB, and ACTN1. In some embodiments, the microarray comprises FLJ10517, HCAP-G, CDKN3, STK6, FOXM1, FLJ10540, TNFRSF6B, HBP17, C1QDC1, TUBG1, FLJ10036, RRM2, ACTB, ACTN1, and EPHA2. In some embodiments, the microarray comprises FLJ10517, HCAP-G, CDKN3, STK6, FOXM1, FLJ10540, TNFRSF6B, HBP17, C1QDC1, TUBG1, FLJ10036, RRM2, ACTB, ACTN1, EPHA2, and TRIP13. In some embodiments, the microarray comprises FLJ10517, HCAP-G, CDKN3, STK6, FOXM1, FLJ10540, TNFRSF6B, HBP17, C1QDC1, TUBG1, FLJ10036, RRM2, ACTB, ACTN1, EPHA2, TRIP13, and CKS2. In some embodiments, the microarray comprises FLJ10517, HCAP-G, CDKN3, STK6, FOXM1, FLJ10540, TNFRSF6B, HBP17, C1QDC1, TUBG1, FLJ10036, RRM2, ACTB, ACTN1, EPHA2, TRIP13, CKS2, and VRK1.

In some embodiments, the microarray comprises FLJ10517, HCAP-G, CDKN3, STK6, FOXM1, FLJ10540, TNFRSF6B, HBP17, C1QDC1, TUBG1, FLJ10036, RRM2, ACTB, ACTN1, EPHA2, TRIP13, CKS2, VRK1, and DUSP4. In some embodiments, the microarray comprises FLJ10517, HCAP-G, CDKN3, STK6, FOXM1, FLJ10540, TNFRSF6B, HBP17, C1QDC1, TUBG1, FLJ10036, RRM2, ACTB, ACTN1, EPHA2, TRIP13, CKS2, VRK1, DUSP4, and EIF4A1. In some embodiments, the microarray comprises FLJ10517, HCAP-G, CDKN3, STK6, FOXM1, FLJ10540, TNFRSF6B, HBP17, C1QDC1, TUBG1, FLJ10036, RRM2, ACTB, ACTN1, EPHA2, TRIP13, CKS2, VRK1, DUSP4, EIF4A1, and SERPINE2.

In some embodiments, the expression profile of one or more genes or a set of genes may allow an individual to determine the prognosis of the patient and/or the likelihood that an individual patient to whom the clinical test is administered will respond to a specific form of therapy, such as, for example, chemotherapy. In some embodiments, the pattern may be different for different chemotherapy regimens. These distinctions, which distinguish a patient who will respond to chemotherapy from those who will not, may be observed regardless of the prognosis of the patient, and may be particularly useful in identifying patients with a poor prognosis, late stage, or aggressive form of breast cancer who will respond to chemotherapy from those who will not.

Identification of patients who will respond to various forms of chemotherapy may be carried out using the tests and methods described herein. For example, in some embodiments, the test may identify patients who will respond to alkylating agents including for example, nitrogen mustards such as mechlorethamine (nitrogen mustard), chlorambucil, cyclophosphamide (Cytoxan®), ifosfamide, and melphalan; nitrosoureas such as streptozocin, carmustine (BCNU), and lomustine; alkyl sulfonates such as busulfan; triazines such as dacarbazine (DTIC) and temozolomide (Temodar®); and ethylenimines, such as, thiotepa and altretamine (hexamethylmelamine); and the like. In other embodiments, a patient's response to antimetabolites including but not limited to 5-fluorouracil (5-FU), capecitabine (Xeloda), 6-mercaptopurine (6-MP), methotrexate, gemcitabine (Gemzar®), cytarabine (Ara-C®), fludarabine, and pemetrexed (Alimta®) and the like may be tested, and in still other embodiments, efficacy of anthracyclines such as, for example, daunorubicin, doxorubicin (Adriamycin®), epirubicin, and idarubicin and other anti-tumor antibiotics including, for example, actinomycin-D, bleomycin, and mitomycin-C may be tested. In yet other embodiments, the clinical test may be directed to identifying patients who will respond to topoisomerase I inhibitors such as topotecan and irinotecan (CPT-11) or topoisomerase II inhibitors such as etoposide (VP-16), teniposide, and mitoxantrone, and in further embodiments, the clinical test may be configured to determine the patients response to corticosteroids such as, but not limited to, prednisone, methylprednisolone (Solumedrol®) and dexamethasone (Decadron®). In some embodiments, the test may be configured to identify patients who will respond to mitotic inhibitors including, for example, taxanes such as paclitaxel (Taxol®) and docetaxel (Taxotere); epothilones such as ixabepilone (Ixempra®); vinca alkaloids such as vinblastine (Velban®), vincristine (Oncovin®), and vinorelbine (Navelbine); and estramustine (Emcyt®). Without wishing to be bound by theory, a clinician may be capable of determining the efficacy of any or all of the chemotherapy agents identified above or known or developed in the future based on the expression profile derived from a microarray having probes for same marker genes, and in certain embodiments, a clinician may be capable of distinguishing the efficacy of individual forms of chemotherapy based on microarrays having probes for the same marker genes.

Some embodiments of the invention are also directed to methods for using the tests of the embodiments described above. For example, various embodiments, may include the steps of obtaining tissue samples from a patient. In some embodiments the methods comprise isolating genetic material and/or proteins from the tissue samples. In some embodiments a method comprises determining the expression levels of one or more markers from the isolated or non-isolated genetic material. In some embodiments, a method comprises determining a genetic profile (e.g. 3D-signature) from the expression levels of the one or more markers. In some embodiments, a method comprises providing treatment to patients whose expression profile matches or nearly matches a predetermined expression profile that indicates that a patient will respond to the treatment. Determining the expression levels of one or more marker genes may be carried out by any method such as, but not limited to, the methods described herein. For example, in some embodiments, the expression levels of one or more marker genes may be measured using polymerase chain reaction (PCR), enzyme-linked immunosorbent assay (ELISA), magnetic immunoassay (MIA), flow cytometry, microarrays, or any such methods known in the art. In some embodiments, one or more microarray may be used to measure the expression level of one or more marker genes, and in some embodiments, the method may further include the steps of labeling the isolated genetic material or proteins and applying the labeled isolated genetic material or proteins to a microarray configured to identify patients who will respond to a form of treatment.

The steps described herein can be used either alone or in combination with any other step described herein. In some embodiments, the steps are performed by the same entity or individual or by different entities or individuals.

In some embodiments, the step of obtaining tissue samples from a patient may be carried out by any method. For example, in some embodiments, the tissue sample may be obtained by excising tissue from the patient during surgery, and in other embodiments, the tissue sample may be obtained by aspirating tissue or cells from a patient prior to surgery such as a tumor. In some embodiments, the tissue extracted may be tumor tissue excised during a tumorectomy or an invasive biopsy of a tumor, or aspirated from a tumor as a less invasive means to biopsy the tumor. In some embodiments, the tissue sample may be of diseased tissue. In some embodiments, the tissue sample may be from normal healthy tissue, and in some embodiments, the tissue sample may include one or more tissue samples from diseased or tumor tissue and normal healthy tissue.

Similarly, the step of isolating genetic material and/or protein may be carried out by any method known in the art. For example, numerous methods for extracting proteins from a tissue sample are known in the art, and any such method may be used in embodiments of the invention. Similarly, numerous methods and kits for extracting DNA and/or RNA (e.g. mRNA) from a tissue sample are known in the art and may be used to isolate genetic material or any portion thereof from the tissue sample. In some embodiments, the step of isolating genetic material from the tissue sample may further include the step of amplifying the genetic material. For example, in some embodiments, mRNA may be isolated from the tissue sample using a known method, and the isolated mRNA may be amplified using PCR or RT-PCR to produce cDNA or cRNA. Methods for amplifying mRNA using such methods are well known in the art and any such method may be used.

Having isolated the proteins and/or genetic material from the tissue sample and, in some embodiments, having amplified the isolated genetic material or a portion thereof, the resulting protein or genetic material may be labeled using any method. For example, in some embodiments, genetic material may be labeled using biotin, and in other embodiments, the genetic material may be labeled using radio-labeled nucleotides or fluorescent label such as a fluorescent nanoparticles or quantum dots. Proteins can be labeled using similar techniques. As above, methods for labeling genetic materials and proteins are well known in the art and any such methods may be used in embodiments of the invention.

The step of applying the labeled proteins or genetic material to a microarray may be carried by any method known in the art. In general, such methods may include the steps of preparing a solution containing the labeled protein or genetic material, contacting the microarray with the solution containing the labeled protein or genetic material, and allowing the labeled protein or genetic material to bind or hybridize to probes associated with the microarray. The various steps associated with applying the labeled proteins or genetic materials to a microarray are well known in the art and can be carried out using any such method. Additionally, in some embodiments, the step of allowing the labeled protein or genetic material to bind or hybridize to probes associated with the microarray may include an incubation step wherein the microarray is immersed in the solution for a period of time from, for example, 15 minutes to 3, 4, 5, or 6 to 12 hours to allow adequate hybridization. In certain embodiments, the incubation step may be carried out at room temperature, and in other embodiments, the incubation step may be carried out at a reduced temperature or an increased temperature as compared to room temperature which may facilitate binding or hybridization.

The step of developing the genetic profile from the microarray may include any number of steps necessary to observe the label associated with labeled protein or genetic material and quantify the intensity of the signal derived from the labeled protein or genetic material. For example, in some embodiments in which biotin is used to label genetic material, the step of developing the genetic profile of the microarray may include the step of washing the microarray with streptavidin, and/or in some embodiments, additionally washing the microarray with an anti-streptavidin biotinylated antibody to stain the microarray, or any combination thereof. The hybridized labeled genetic material may then be observed and the intensity of the signal quantified using fluormetric scanning. In some embodiments in which the protein or genetic material is labeled with a radio-nucleotide, observing and quantifying the intensity can be carried out using emulsion films such as X-ray film or any manner of scintillation counter or phosphorimager. Numerous methods for performing such techniques are known in the art and may be used. In some embodiments, nanoparticles or quantum dots may be observed and quantified by exciting the quantum dot under light of a specific wavelength and viewing the microarray using, for example, a CCD camera. The intensity of signal derived from images of the microarrays can then be determined using a computer and imaging software. Such methods are well known and can be carried out using numerous techniques.

In some embodiments, developing the genetic profile may further include comparing the intensities of the signal from one or more probes for genetic markers on the microarray with microarrays derived from normal healthy tissue which may or may not be from the same patient or standard intensities which reflect compiled genetic profiles data from similar clinical tests for numerous individuals having the subject disease such as cancer or breast cancer. In such embodiments, modulated expression of a particular gene may be evident by an increase or a decrease in signal from a probe associated with the particular gene, and an increase or a decrease in a specific gene may by indicative of a genetic profile for a patient who will respond well to a specific form of treatment. For example, a patient whose expression profile exhibits an increase in expression in the RRM2 (ribonucleotide reductase M2 polypeptide) gene over the median intensity for that gene of all patients having breast cancer whose expression profile was determined using the same clinical test or microarray may have a greater likelihood of responding to treatment using chemotherapy, such as, taxane therapy. In some embodiments, the change in intensity may be significant and obvious, for example, a dramatic change (10-fold) in intensity for one or more genetic marker may be observed based on the average expression profile. In some embodiments, a change in intensity may be reflected in about 10% to about 20% reduction in intensity for one or more genetic markers. Without wishing to be bound by theory, detecting this change in intensity and correlating it with a therapeutic sensitivity of an individual, may provide a sensitive, fast, and reproducible means for identifying therapeutic agents that will effectively treat the disease and/or tailoring specific therapeutic regimens for individual patients that increase their chances of alleviating or curing the diseased state. For example, in some embodiments, markers in tests for breast cancer may accurately identify individuals that will respond to taxane treatment over breast cancer patients who will not respond to such treatment by detecting a difference in intensity for one or more genetic markers with a p-value from about 0.001 to about 0.00001, and in other embodiments about 0.0001.

Having developed the expression profile of a patient based on the microarray of the clinical test and having determined the therapeutic sensitivity of the patient, the patient may be treated using the appropriate therapeutic agent such as one or more of the chemotherapy agents described above. In some embodiments, the therapeutic agent identified may be administered alone. In some embodiments, the therapeutic agent identified may be administered as part of a course of treatment that may include one or more other forms of treatment. For example, in some embodiments, a therapeutic agent identified using the methods of embodiments of the invention may be provided as a form of neoadjuvant therapy for cancer. In some embodiments, the identified therapeutic agent may be administered to the patient before radiation or surgery to reduce the size of a tumor, and reducing the size of the tumor may reduce the amount of tissue removed during surgery. For example, in breast cancer, neoadjuvant therapy has been shown to increase the likelihood of a successful lumpectomy, which conserves breast tissue while removing the tumor reducing the need for a mastectomy in which one or both breasts are completely removed. Thus, embodiments of the method may include the steps of administering a therapeutic agent identified using the clinical test alone or in combination with one or more other forms of therapy, and/or the step of administering the therapeutic agent identified as a form of neoadjuvant therapy for cancer, such as but not limited to breast cancer.

In some embodiments, kits are provided for determining an appropriate therapeutic agent to treat a disease that includes the clinical test of embodiments described above, and one or more additional elements for preparing an expression profile from a tissue sample using the clinical test. For example, in some embodiments, a kit may include an apparatus for collecting a tissue sample, components for determining the expression levels of one or more genes associated with the disease, labels, reagents, other materials necessary to determine the expression profile, instructions for identifying a therapeutic agent based on the expression profile, or any combination thereof. Determining the expression levels of one or more marker genes may be carried out by any method such as polymerase chain reaction (PCR), enzyme-linked immunosorbent assay (ELISA), magnetic immunoassay (MIA), microarrays, or any such methods known in the art, and the contents of the kits of various embodiments may vary based on the method utilized. For example, in some embodiments PCR may be the method for determining the expression level of one or more marker genes, and the kit may include single-stranded DNA primers which facilitate amplification of a marker gene. In some embodiments, ELISA or MIA based kits may include antibodies directed to a specific protein and/or fluorescent or magnetic probes. In some embodiments, one or more microarray may be used to measure the expression level of one or more marker genes, and such kits may include one or more microarrays having probes to specific marker genes.

Any apparatus for collecting a tissue sample may be used. For example, in some embodiments, the apparatus may be a needle and/or syringe used to aspirate cells or tissue from diseased tissue such as a tumor. In some embodiments, the kit may be include a scalpel or other instrument for obtaining a tissue sample. In some embodiments, the kit may include a combination of apparatuses that may be used to obtain a tissue sample. In further embodiments, the kit may include an instruction describing the use of another commercially available apparatus to obtain a tissue sample.

In some embodiments, one or more labels for the protein or genetic material may also be provided in the kit. For example, kits of various embodiments may include a label, such as biotin, the reagents and materials necessary to perform biotinylation, a radio-label or radio-labeled nucleotide, reagents and materials necessary to incorporate a radioactive label into isolated protein or genetic materials, fluorescent label and reagents, materials necessary to fluorescently label the isolated protein or genetic material, nanoparticles, nanocrystals, or quantum dots, reagents and materials necessary to label the isolated protein or genetic material with nanoparticles, nanocrystals, or quantum dots, or any combination thereof.

Numerous reagents may be provided in the kits of embodiments of the invention including, for example, reagents necessary for tissue sample acquisition and storage, reagents necessary for protein and/or genetic material isolation, reagents necessary for labeling, reagents necessary to perform PCR, ELISA, MIA, or using a microarray, reagents for producing a solution used to apply labeled protein or genetic material to the microarray, reagents necessary for developing the microarray, reagents used in conjunction with observing, analyzing or quantifying the expression levels, the expression profile, reagents for the storage of the microarray following processing, and the like and any combination thereof. In some embodiments, the kit may include vials of such reagents in solution arranged and labeled to allow ease of use. In some embodiments, the kit may include the component parts of the various reagents which may be combined with a solvent such as, for example, water to create the reagent. The component parts of some embodiments may be in solid or liquid form where such liquids are concentrated to reduce the size and/or weight of the kit thereby improving portability. In some embodiments, the various reagents necessary to use the clinical test of various embodiments may be supplied by providing the recipe and or instructions for making the reagents or exemplary reagents that may be substituted by other commonly used similar reagents.

In some embodiments, the kits of the invention may include materials necessary to develop a microarray. For example, in some embodiments, the kit may include an apparatus for holding the microarray and/or sealing at least an area surrounding the microarray to ensure that solutions containing labeled proteins or genetic material remain in contact with the microarray for a sufficient period of time to allow adequate binding or hybridization. In some embodiments, the kit may include apparatuses for ease of handling the microarray during development. In some embodiments, the kits of the invention may include a device for observing the labeled protein or genetic material on the microarray and/or quantifying the intensity of the signal generated by the labeled protein or genetic material. In some embodiments, the kit may include exemplary data, charts, and intensity comparison markers. In some embodiments, these or other similar materials may be provided in written form, and in other such embodiments, these or other similar materials may be provided on a computer readable medium, such as, but not limited, a flash drive, CD, DVD, Blue-Ray disc, and the like. In some embodiments, various materials may be provided through an internet website accessible to kit purchasers. Similarly, instructions for using the kit and any materials supplied with the kit may be provided with purchase of the kit in written form, on a computer readable medium, or on a similar internet website.

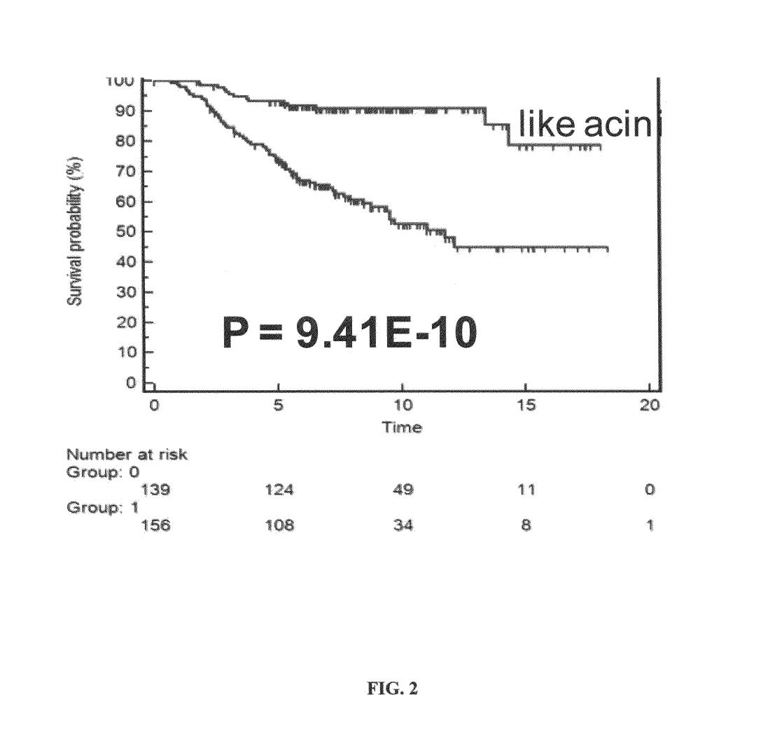

In some embodiments, embodiments of the present invention are directed to a 3D gene signature that accurately predicts the chemotherapeutic response outcome in breast cancer. In addition, the 3D signature can be an indicator for breast cancer prognosis. An example of this was seen in the 3 independent datasets with over 700 breast cancer patients (see, for example, FIG. 2). The 3D signature can be created by analyzing the expression of the one or more markers or any combination thereof described herein.

Table 1 shows a multivariable proportional-hazards analysis of 10-year survival risk. It indicates that the 3D signature is a strong independent factor to predict breast cancer clinical outcome. Results calculated using dataset of van de Vijver, et al., using overall survival as endpoint.

TABLE 1

| | Hazard ratio | |
|---|---|---|
| | (95% CI)^{a} | P-value |
| Age (per 10 year increment) | 0.62 (0.44 to 0.88) | 0.008 |
| Tumor diameter (per cm) | 1.33 (1.04 to 1.69) | 0.023 |
| ER (positive vs negative) | 0.55 (0.34 to 0.90) | 0.018 |
| Lymph node status (per | 1.07 (0.96 to 1.20) | 0.234 |
| positive node) | | |
| Chemotherapy | 0.69 (0.38 to 1.26) | 0.234 |
| Mastectomy | 1.05 (0.63 to 1.73) | 0.864 |
| BIOARRAY signature | 4.43 (2.32 to 8.46) | <0.00001 |

Martin et al. PLoS One 2008

In some embodiments, methods for predicting therapeutic response to breast cancer are provide. In some embodiments, the method comprises isolating genetic material from the diseased tissue samples of a patient with breast cancer. In some embodiments, the method comprises developing a genetic profile from the marker genes. In some embodiments, the method comprises determining the subtype of breast cancer in the patient based on the genetic profile. In some embodiments, the method comprises providing treatment to patients whose expression profile matches or nearly matches a predetermined subtype profile that indicates that a patient will respond to the treatment.

In some embodiments, the genetic profile comprises determining the expression levels of one or more markers. The expression levels can be determined as described herein or with another method. In some embodiments, the genetic profile and the related expression levels are transformed into a predictive score. In some embodiments, the predictive score is used to predict response to therapy. The response can be where the cancer is responsive or non-responsive to a therapy. In some embodiments, the predictive score is used to predict prognosis of a subject.

In some embodiments, the genetic profile from the marker genes is referred to as a 3D Signature. In certain embodiment, the 3D signature is simply referred to as “signature”. Unlike most cancer signatures that have been selected by using supervised methods and a specific patient training set, the 3D Signature was selected using a cell culture model that accurately recapitulates the normal process of breast acini formation and growth arrest. Since it is not linked to a particular patient set, the signature more accurately classifies diverse patient subsets than traditionally discovered signatures. This advantage makes the 3D signature a favored signature for predictive response to therapy and/or prognosis.

In some embodiments a kit is provided for testing therapeutic sensitivity of diseased tissue. In some embodiments, the method comprises components for identifying the expression profile of a tissue sample having probes to a specific set of genes or proteins associated with the disease; labels, reagents, other materials or instructions for labeling and preparing reagents and other materials necessary to develop a expression profile of one or more marker genes, or any combination thereof.

In some embodiments, the 3D signature, which includes the expression levels of one or more markers is interpreted by using logistic regression. Logistic regression is a form of regression which is used when the dependent is a dichotomy and the independents are of any type. Logistic regression can be used to predict a dependent variable on the basis of continuous and/or categorical independents and to determine the effect size of the independent variables on the dependent; to rank the relative importance of independents; to assess interaction effects; and to understand the impact of covariate control variables. The impact of predictor variables is usually explained in terms of odds ratios. Logistic regression applies maximum likelihood estimation after transforming the dependent into a logit variable (the natural log of the odds of the dependent occurring or not). In this way, logistic regression estimates the odds of a certain event occurring. Note that logistic regression calculates changes in the log odds of the dependent, not changes in the dependent itself.

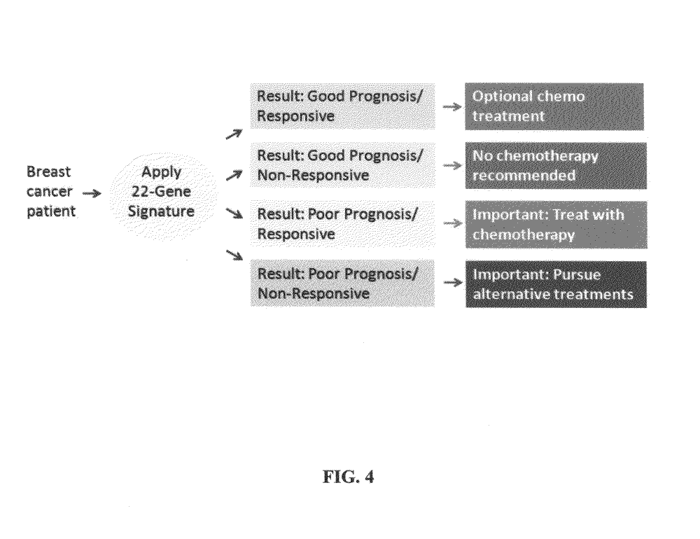

In some embodiments, the gene expression levels of 3D-signature can be successfully used to classify breast cancer patients by disease prognosis. Embodiments of the present invention are directed to the ability of the 3D signature to predict response to chemotherapy in breast cancer. While prognosis divides patients into two classes, chemotherapy response is expected to subdivide each of these two classes into an additional two classes resulting in a total of 4 classes: 1-good prognosis/chemo responsive, 2-good prognosis/chemo non-responsive; 3-poor prognosis/chemo responsive and 4-good prognosis/chemo non-responsive (FIG. 4).

In some embodiments, the method comprises transforming the 3D signature into a predictive score. In some embodiments, the kit comprises components for receiving a sample. In some embodiments, the sample can then be processed.

In some embodiments, the present invention provides a computer implemented method for scoring a first sample obtained from a subject. In some embodiments, the method comprises obtaining a first dataset associated with a first sample. In some embodiments, the dataset comprises expression data for at least one marker set. The marker set can be any marker set described herein. In some embodiments, the marker set comprises expression data for FLJ10517, HCAP-G, CDKN3, STK6, FOXM1, FLJ10540, TNFRSF6B, HBP17, C1QDC1, TUBG1, FLJ10036, RRM2, ACTB, ACTN1, EPHA2, TRIP13, CKS2, VRK1, DUSP4, EIF4A1, SERPINE2, or ODC1, and any combination thereof. In some embodiments, the marker set comprises expression data for FLJ10517, HCAP-G, CDKN3, STK6, FOXM1, FLJ10540, TNFRSF6B, HBP17, C1QDC1, TUBG1, FLJ10036, RRM2, ACTB, ACTN1, EPHA2, TRIP13, CKS2, VRK1, DUSP4, EIF4A1, SERPINE2, and ODC1. In some embodiments, the marker set comprises expression data for FLJ10517 and HCAP-G. In some embodiments, the marker set comprises expression data for FLJ10517, HCAP-G, and CDKN3. In some embodiments, the marker set comprises expression data for FLJ10517, HCAP-G, CDKN3, and STK6. In some embodiments, the marker set comprises expression data for FLJ10517, HCAP-G, CDKN3, STK6, and FOXM1. In some embodiments, the marker set comprises expression data for FLJ10517, HCAP-G, CDKN3, STK6, FOXM1, and FLJ10540. In some embodiments, the marker set comprises expression data for FLJ10517, HCAP-G, CDKN3, STK6, FOXM1, FLJ10540, and TNFRSF6B. In some embodiments, the marker set comprises expression data for FLJ10517, HCAP-G, CDKN3, STK6, FOXM1, FLJ10540, TNFRSF6B, and HBP17. In some embodiments, the marker set comprises expression data for FLJ10517, HCAP-G, CDKN3, STK6, FOXM1, FLJ10540, TNFRSF6B, HBP17, and C1QDC1. In some embodiments, the marker set comprises expression data for FLJ10517, HCAP-G, CDKN3, STK6, FOXM1, FLJ10540, TNFRSF6B, HBP17, C1QDC1, and TUBG1. In some embodiments, the marker set comprises expression data for FLJ10517, HCAP-G, CDKN3, STK6, FOXM1, FLJ10540, TNFRSF6B, HBP17, C1QDC1, TUBG1, and FLJ10036. In some embodiments, the marker set comprises expression data for FLJ10517, HCAP-G, CDKN3, STK6, FOXM1, FLJ10540, TNFRSF6B, HBP17, C1QDC1, TUBG1, FLJ10036, and RRM2. In some embodiments, the marker set comprises expression data for FLJ10517, HCAP-G, CDKN3, STK6, FOXM1, FLJ10540, TNFRSF6B, HBP17, C1QDC1, TUBG1, FLJ10036, RRM2, and ACTB. In some embodiments, the marker set comprises expression data for FLJ10517, HCAP-G, CDKN3, STK6, FOXM1, FLJ10540, TNFRSF6B, HBP17, C1QDC1, TUBG1, FLJ10036, RRM2, ACTB, and ACTN1. In some embodiments, the marker set comprises expression data for FLJ10517, HCAP-G, CDKN3, STK6, FOXM1, FLJ10540, TNFRSF6B, HBP17, C1QDC1, TUBG1, FLJ10036, RRM2, ACTB, ACTN1, and EPHA2. In some embodiments, the marker set comprises expression data for FLJ10517, HCAP-G, CDKN3, STK6, FOXM1, FLJ10540, TNFRSF6B, HBP17, C1QDC1, TUBG1, FLJ10036, RRM2, ACTB, ACTN1, EPHA2, and TRIP13. In some embodiments, the marker set comprises expression data for FLJ10517, HCAP-G, CDKN3, STK6, FOXM1, FLJ10540, TNFRSF6B, HBP17, C1QDC1, TUBG1, FLJ10036, RRM2, ACTB, ACTN1, EPHA2, TRIP13, and CKS2. In some embodiments, the marker set comprises expression data for FLJ10517, HCAP-G, CDKN3, STK6, FOXM1, FLJ10540, TNFRSF6B, HBP17, C1QDC1, TUBG1, FLJ10036, RRM2, ACTB, ACTN1, EPHA2, TRIP13, CKS2, and VRK1. In some embodiments, the marker set comprises expression data for FLJ10517, HCAP-G, CDKN3, STK6, FOXM1, FLJ10540, TNFRSF6B, HBP17, C1QDC1, TUBG1, FLJ10036, RRM2, ACTB, ACTN1, EPHA2, TRIP13, CKS2, VRK1, and DUSP4. In some embodiments, the marker set comprises expression data for FLJ10517, HCAP-G, CDKN3, STK6, FOXM1, FLJ10540, TNFRSF6B, HBP17, C1QDC1, TUBG1, FLJ10036, RRM2, ACTB, ACTN1, EPHA2, TRIP13, CKS2, VRK1, DUSP4, and EIF4A1. In some embodiments, the marker set comprises expression data for FLJ10517, HCAP-G, CDKN3, STK6, FOXM1, FLJ10540, TNFRSF6B, HBP17, C1QDC1, TUBG1, FLJ10036, RRM2, ACTB, ACTN1, EPHA2, TRIP13, CKS2, VRK1, DUSP4, EIF4A1, and SERPINE2.

In some embodiments, the method comprises determining, by a computer processor, a first score from the first dataset that comprises the market set expression data using an interpretation function, wherein the first score is predictive of response to therapy in a subject and/or the prognosis of the subject. In some embodiments, the interpretation function is based upon a predictive model. The predictive model can be predict response to a treatment or the prognosis of a subject.

In some embodiments, a computer comprises at least one processor coupled to a chipset. In some embodiments, also coupled to the chipset are a memory, a storage device, a keyboard, a graphics adapter, a pointing device, and/or a network adapter. A display can also be coupled to the graphics adapter. In some embodiments, the functionality of the chipset is provided by a memory controller hub and an I/O controller hub. In some embodiments, the memory is coupled directly to the processor instead of the chipset.

The storage device can be any device capable of holding data, like a hard drive, compact disk read-only memory (CD-ROM), DVD, Blue-Ray, HD Disc, or a solid-state memory device. The memory holds instructions and data used by the processor. The pointing device may be a mouse, track ball, or other type of pointing device, and is used in combination with the keyboard to input data into the computer system. The graphics adapter displays images and other information on the display. The network adapter couples the computer system to a local or wide area network.

Additionally, a computer can have different and/or other components than those described herein. In addition, the computer can lack certain components. Moreover, the storage device can be local and/or remote from the computer (such as embodied within a storage area network (SAN)). In some embodiments, the computer is adapted to execute computer program modules for providing the functionality described herein. As used herein, the term “module” refers to computer program logic utilized to provide the specified functionality. Thus, a module can be implemented in hardware, firmware, and/or software. In one embodiment, program modules are stored on the storage device, loaded into the memory, and executed by the processor. The computer can be adapted to, for example, determine the expression data process the data in conjunction with algorithm's described herein. The computer can also provide a predictive score utilizing the expression data and other clinical factors as described herein.

In some embodiments, the dataset comprises a clinical factor. The clinical factor can be for example, but not limited to, age, gender, neutrophil count, ethnicity, race, disease duration, diastolic blood pressure, systolic blood pressure, a family history parameter, a medical history parameter, a medical symptom parameter, height, weight, a body-mass index, resting heart rate, and smoker/non-smoker status, subtype of breast cancer, and the like. In some embodiments, the dataset comprises other clinical factors including, but not limited, ER status, HER2 status, tumor size, tumor grade, and patient node status. Other examples of clinical factors include, but are not limited to, whether the subject has diabetes, whether the subject has an inflammatory condition, whether the subject has an infectious condition, whether the subject is taking a steroid, whether the subject is taking an immunosuppressive agent, and/or whether the subject is taking a chemotherapeutic agent or has previously been treated with a cancer therapeutic or other chemotherapeutic agent. The clinical factors can be determined by a clinician (e.g. physician). For example, the age can be the patient age before chemotherapy treatment. The tumor grade can be referred to as tumor BMN grade (1, 2 or 3) before chemotherapy treatment. The ER-status can be clinically determined status and, can be for example, ER-negative=0 or ER-positive=1. The node status can be, for example, number of positive nodes before chemotherapy treatment. In some embodiments, the tumor-size can be the size (e.g. mm or cm) before chemotherapy treatment. In some embodiments, the expression data were measured by microarray gene expression levels.

In some embodiments, the predictive model is a logistic regression model.

In some embodiments, obtaining the dataset comprises obtaining the sample and processing the sample to experimentally determine the first dataset. The dataset that can comprise the expression data of the marker set or sets described herein. The data set can be experimentally determined by, for example, using a microarray or quantitative amplification method such as, but not limited to, those described herein. In some embodiments, obtaining a dataset associated with a sample comprises receiving the dataset from a third party that has processed the sample to experimentally determine the dataset.

In some embodiments, the method comprises classifying the sample according to the predictive score that is determined. The sample can be classified as responsive, non-responsive, poor prognosis, good prognosis, undeterminable prognosis, and the like. In some embodiments, wherein the sample comprises RNA extracted from peripheral blood cells or circulating breast epithelial cells. In some embodiments, the expression data are derived from hybridization data (e.g. using a microarray). In some embodiments, the expression data are derived from polymerase chain reaction data. In some embodiments, the expression data are derived from RT-PCR data.

In some embodiments, the present invention provides a system for predicting response to therapy and/or prognosis. In some embodiments, the system comprises a storage memory for storing a dataset derived from or associated with a sample obtained from a subject. As described herein, the dataset can comprise expression data. The expression data can comprise one or more markers, marker sets, or combinations of markers as described herein. In some embodiments, the system comprises a processor. In some embodiments, the processor can be communicatively coupled to the storage memory for determining a score with an interpretation function wherein the score is predictive response to therapy and/or prognosis of the subject.

In some embodiments, the interpretation function can be a function produced by a predictive model. The predictive model can be, for example, a logistic regression model. An interpretation function can created by more than one predictive model.

In some embodiments, the predictive model performance can be characterized by an area under the curve (AUC). In some embodiments, the predictive model performance is characterized by an AUC ranging from 0.68 to 0.70. In some embodiments, the predictive model performance is characterized by an AUC ranging from 0.70 to 0.79. In some embodiments, the predictive model performance is characterized by an AUC ranging from 0.80 to 0.89. In some embodiments, the predictive model performance is characterized by an AUC ranging from 0.90 to 0.99.

In some embodiments, the interpretation function comprises an algorithm to produce the predictive score. In some embodiments, the interpretation function comprises at least one of an age term, a grade term, an ER-status term, node-status term, tumor-size term, and one or more gene marker terms including, but not limited to the genes described herein.

In some embodiments, the interpretation function comprises an algorithm where the predictive score is determined according to a predictive model, such as but not limited to logistical regression. In some embodiments, the predictive score (e.g. score) is determined by the following:

score=log(p/1−p)=0.2266+0.0295*age−0.5074*grade+0.0248*ER−status+0.0114*node−status+0.2352*tumor−size+0.2577*CDH3+0.0551*ESR1−0.0876*HER2−0.5976*ODC1−0.2474*TRIP13−0.1695*SERPINE2+0.8003*FGFBP;

score=log(p/1−p)=0.850+1.215*EPHA2+2.070*ER−status−0.356*HER2−status−0.462*OCD1−0.196*SERPINE2;

score=log(p/1−p)=7.399−4.143*EPHA2+3.168*FGFBP1−1.264*tumor grade−0.347*HER2−status+0.947*node−status;

score=log(p/1−p)=−2.518−18.864*ESR1+0.997*tumor size+1.556*TUBG; or

log(p/1−p)=1.441+2.036*ESR1−0.716*ODC1

In some embodiments, the scores are determined depending upon the cancer subtype or physical characteristics of the cancer. In some embodiments, the score that determined using any of the algorithms described herein is based upon ER status, Luminal B status, or the cancer is characterized as basal like. In some embodiments, the predictive score is an average of one or more scores as determined herein.

In some embodiments, the score for an ER-positive cancer is selected from the group consisting of:

score=log(p/1−p)=0.2266+0.0295*age−0.5074*grade+0.0248*ER−status+0.0114*node−status+0.2352*tumor−size+0.2577*CDH3+0.0551*ESR1−0.0876*HER2−0.5976*ODC1−0.2474*TRIP13−0.1695*SERPINE2+0.8003*FGFBP; log(p/1−p)=0.850+1.215*EPHA2+2.070*ER−status−0.356*HER2−status−0.462*OCD 1−0.196*SERPINE2; or

score=log(p/1−p)=7.399−4.143*EPHA2+3.168*FGFBP1−1.264*tumor grade−0.347*HER2−status+0.947*node−status.

In some embodiments, the score for an ER-negative cancer is selected from the group consisting of: log(p/1−p)=0.2266+0.0295*age−0.5074*grade+0.0248*ER−status+0.0114*node−status+0.2352*tumor−size+0.2577*CDH3+0.0551*ESR1−0.0876*HER2−0.5976*ODC1−0.2474*TRIP13−0.1695*SERPINE2+0.8003*FGFBP; or score=log(p/1−p)=0.850+1.215*EPHA2+2.070*ER−status−0.356*HER2−status−0.462*OCD1−0.196*SERPINE2.

In some embodiments, the score for a luminal B cancer is selected from the group consisting of: score=log(p/1−p)=0.2266+0.0295*age−0.5074*grade+0.0248*ER−status+0.0114*node−status+0.2352*tumor−size+0.2577*CDH3+0.0551*ESR1−0.0876*HER2−0.5976*ODC1−0.2474*TRIP13−0.1695*SERPINE2+0.8003*FGFBP; or log(p/1−p)=0.850+1.215*EPHA2+2.070*ER−status-0.356*HER2−status−0.462*OCD 1−0.196*SERPINE2.

In some embodiments, the score for a basal like cancer is selected from the group consisting of: score=log(p/1−p)=0.2266+0.0295*age−0.5074*grade+0.0248*ER−status+0.0114*node−status+0.2352*tumor−size+0.2577*CDH3+0.0551*ESR1−0.0876*HER2−0.5976*ODC1−0.2474*TRIP13−0.1695*SERPINE2+0.8003*FGFBP.

In some embodiments, the score for a HER2-positive cancer is selected from the group consisting of: score=log(p/1−p)=0.2266+0.0295*age−0.5074*grade+0.0248*ER−status+0.0114*node−status+0.2352*tumor−size+0.2577*CDH3+0.0551*ESR1−0.0876*HER2−0.5976*ODC1−0.2474*TRIP13−0.1695*SERPINE2+0.8003*FGFBP; or score=log(p/1−p)=−2.518−18.864*ESR1+0.997*tumor size+1.556*TUBG.

In some embodiments, the score for a triple negative breast cancer is selected from the group consisting of: score=log(p/1−p)=0.2266+0.0295*age−0.5074*grade+0.0248*ER−status+0.0114*node−status+0.2352*tumor−size+0.2577*CDH3+0.0551*ESR1−0.0876*HER2−0.5976*ODC1−0.2474*TRIP13−0.1695*SERPINE2+0.8003*FGFBP; or score=log(p/1−p)=1.441+2.036*ESR1−0.716*ODC1.

In some embodiments, the score for any cancer is selected from the group consisting of: score=log(p/1−p)=0.2266+0.0295*age−0.5074*grade+0.0248*ER−status+0.0114*node−status+0.2352*tumor−size+0.2577*CDH3+0.0551*ESR1−0.0876*HER2−0.5976*ODC1−0.2474*TRIP13−0.1695*SERPINE2+0.8003*FGFBP; score=log(p/1−p)=0.850+1.215*EPHA2+2.070*ER−status−0.356*HER2−status−0.462*OCD 1−0.196*SERPINE2; score=log(p/1−p)=0.850+1.215*EPHA2+2.070*ER−status−0.356*HER2−status−0.462*OCD1−0.196*SERPINE2.

The score can be determined using any of the interpretation functions described herein. In the functions described herein, the term “CDH3” refers to cadherin 3, “ESR1” refers to estrogen receptor 1, “HER2” refers to Human Epidermal growth factor Receptor 2.

In some embodiments, the score is determined by analyzing markers that are down regulated (expression is lower) during acini formation in 3D culture. Tumors that have a similar gene signature were found to be associated with a prediction that they would respond to treatment. In some embodiments, the response is a response to paclitaxel (Taxol®), 5-fluouracil, doxorubicin (Adriamycin™) and cyclophosphamide (TFAC) chemotherapy. In some embodiments, the ability to predict response and prognosis in breast cancer are overlapping but not synonymous. As shown in the examples, a 22-gene signature (down-regulated late in acini formation) accurately predicted TFAC response across a broad range of breast cancer subtypes and outperformed clinical parameters.

In some embodiments, the score, which can also be referred to as the predictive score has a cut-off value. The cut-off value is a value where when the predictive score is below the cut-off value the predictive score predicts that the cancer will not respond to a treatment or where the predictive score is above the cut-off value the predictive score predicts that the cancer will respond to a treatment. In some embodiments, a cancer is predicted to respond to a treatment when the predictive score is greater than or greater than or equal to the cut-off value. In some embodiments, a cancer is predicted to not to respond to a treatment when the predictive score is less than or less than or equal to the cut-off value. In some embodiments, a cancer is predicted to respond to a treatment when the predictive score is equal to the cut-off value. In some embodiments, a cancer is predicted to not to respond to a treatment when the predictive score is equal to the cut-off value. In some embodiments, the cut-off value is specified. In some embodiments, the specified cut-off value is from about 0.1 to about 0.9, about 0.2 to about 0.8, about 0.3 to about 0.7, about 0.4 to about 0.8, about 0.4 to about 0.7, about 0.4 to about 0.9, about 0.5 to about 0.9, about 0.5 to about 0.7, about 0.5 to about 0.6. In some embodiments, the specified cut-off value is about or exactly 0.1, 0.2, 0.3, 0.4, 0.5, 0.6, 0.7, 0.8, or 0.9. In some embodiments, the specified cut-off value is at least 0.1, 0.2, 0.3, 0.4, 0.5, 0.6, 0.7, 0.8, or 0.9. In some embodiments, the specified cut-off can be different for different types of cancers.

In some embodiments, a method for predicting a response to a treatment as described herein comprises transforming the predictive score into an output that is communicated to a user. The output can be as simple as a message stating that the cancer should be responsive or not responsive. In some embodiments, the output is a statistical analysis of the probability of response to a treatment, which is based upon the predictive score. The output can be communicated by a machine orally, electronically in a message, or on printed matter. In some embodiments, the output is displayed on a screen. Accordingly, in some embodiments, the systems described herein also can comprise a display unit that is communicatively connected to the processor such that the display unit can display the output.

In some embodiments, the interpretation function comprises: score=log(p/1−p)=0.2266+0.0295*age−0.5074*grade+0.0248*ER−status+0.0114*node−status+0.2352*tumor−size+0.2577*CDH3+0.0551*ESR1−0.0876*HER2−0.5976*ODC1−0.2474*TRIP13−0.1695*SERPINE2+0.8003*FGFBP; score=breast cancers version 2: log(p/1−p)=0.850+1.215*EPHA2+2.070*ER−status−0.356*HER2−status−0.462*OCD 1−0.196*SERPINE2; score=log(p/1−p)=7.399−4.143*EPHA2+3.168*FGFBP1−1.264*tumor grade−0.347*HER2−status+0.947*node−status; score=log(p/1−p)=−2.518−18.864*ESR1+0.997*tumor size+1.556*TUBG; score=log(p/1−p)=1.441+2.036*ESR1−0.716*ODC1

In some embodiments, a sample can be characterized as Luminal A when it has high ESR1 and low AURKA; Luminal B when it has high ESR1 and high AURKA; HER2+ when it has high ERBB; Basal-like when it has low ESR1 and high KRT5. The levels are compared to a normal tissue to determine if it is high or low. If the values are greater than found in a normal sample or a matched pair sample it is said to be high. If the values are lower than found in a normal sample or a matched pair sample it is said to be low.

Although the present invention has been described in considerable detail with reference to certain preferred embodiments thereof, other versions are possible. Therefore the spirit and scope of the appended claims should not be limited to the description and the preferred versions contained within this specification. Various aspects of the present invention will be illustrated with reference to the following non-limiting examples.

### EXAMPLES

### Example 1

All results in this study were obtained from the microarray dataset of Hess K R, Anderson K, Symmans W F, et al. Journal of Clinical Oncology 24(26): 4236-44, 2006, contents of which are incorporated by reference herein. In summary, fine-needle aspirates from patients with stage I-III breast cancer were obtained before neoadjuvant combination treatment and response was assessed after chemotherapy. Aspirates were analyzed on Affymetrix HG-U133A microarrays. An additional 145 samples for a total of 278 samples were added to the Gene Expression Omnibus (GEO) resource in 2010 and were also used in this study. Affymetrix Excel files were downloaded from GEO, preprocessed by RMA using GeneSpring, and then genes were normalized to the median expression level. RMA is used to compute gene expression summary values for Affymetrix data by using the Robust Multichip Average expression summary and to carry out quality assessment using probe-level metrics. Replicate and poor quality samples (normalized gene expression standard deviation >0.75) were omitted.

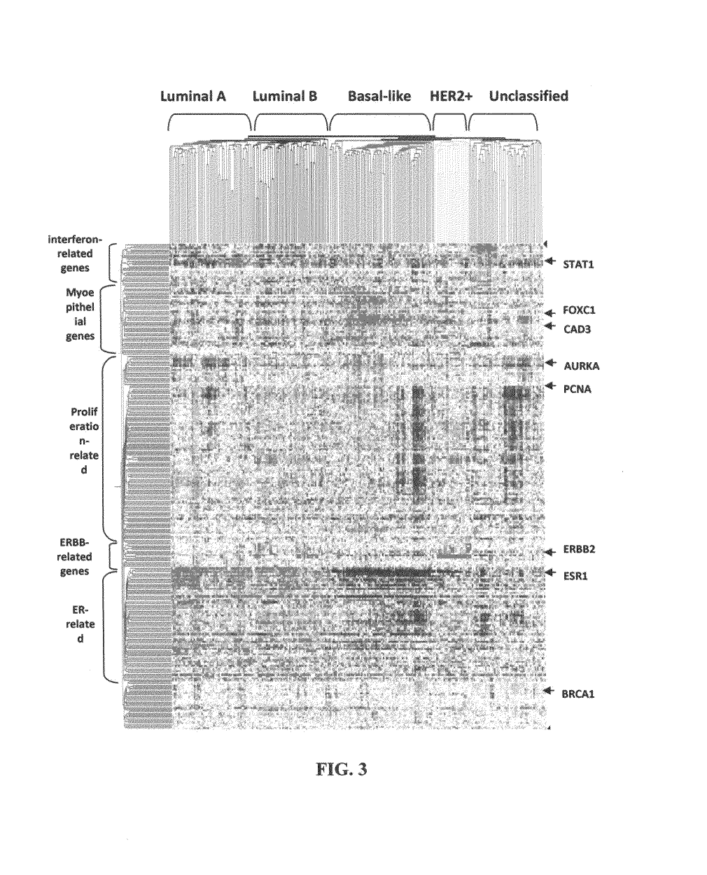

Molecular classes were determined using the intrinsic gene set of 300 genes (Hu et al, 2007). 263 were translated onto Affymetrix HG-U133A GeneChips and expression values organized by hierarchical clustering with a Pearson metric resulting in sample clustering into five classes. Clusters were identified as: Luminal A=high ESR1, low AURKA; Luminal B=high ESR1, high AURKA; HER2+=high ERBB; Basal-like=low ESR1, high KRT5; and Unclassified which was the remaining cluster (FIG. 3).

In this study, the 3D signature is applied using a logistic regression. Logistic regression is used to predict the probability of occurrence of an event by fitting data to a logistic curve, i.e. a common sigmoid (S-shaped) curve. Analyses were performed using SAS software. Results are presented as area under the curve (AUC) statistics, which is a summary statistic that combines sensitivity and specificity into a single measure. AUC=1.0 is a perfect test, 0.9-1.0 is an excellent test, 0.8-0.9 is a very good test, 0.7-0.8 is a good test.

The number of samples for molecular class and response categories of expanded microarray dataset of Hess, et al., 2006 is shown in Table 2.

TABLE 2

| | Actual numbers | | | Percentages | | |
|---|---|---|---|---|---|---|
| | no pCR | pCR | Total | no pCR | pCR | Total |
| Basal-like | 42 | 27 | 69 | 17% | 11% | 29% |
| HER2+ | 8 | 11 | 19 | 3% | 5% | 8% |
| Luminal A | 55 | 1 | 56 | 23% | 0% | 23% |
| Luminal B | 43 | 7 | 50 | 18% | 3% | 21% |
| Unclassified | 43 | 5 | 48 | 18% | 2% | 20% |
| Total | 191 | 51 | 242 | 79% | 21% | 100% |
| ER Negative | 54 | 43 | 97 | 22% | 18% | 40% |
| ER Positive | 137 | 8 | 145 | 57% | 3% | 60% |
| Total | 191 | 51 | 242 | 79% | 21% | 100% |

Table 3 illustrates the results of models built using expression levels of the 22 3D-signature genes. Logistic regression allows for an accurate prediction of response to chemotherapy for a broad range of subtypes of breast cancer. The gray highlighted numbers show the best condition AUC statistic for each tumor classification group listed at the left. For example, for the group “All types”, the best AUC obtained was 0.875, which was obtained with model M5. This model included the following variables: expression levels of the 22 3D-signature genes, breast tumor subtype information, and ER status information. In this case, the model was trained over all tumor subtypes.

TABLE 3

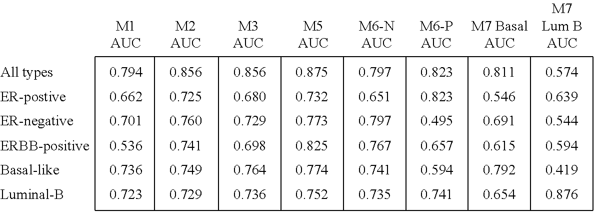

M1: model gene variables (trained over all types)
M2: model includes genes + subtype variable (trained over all types)
M3: model includes genes + ER variable (trained over all types)
M5: model includes genes + subtype and ER variables (trained over all types)
M6: model includes genes + subtype (trained over all ER pos and ER neg separately)
M7: train over subtypes seperately include genes + ER

Models were trained using the criteria indicated above on 80% (194 of 242) samples. The tabulated AUC's are from a standard 5-fold cross validation of the remaining 20% (48 of 242) samples where the 20% hold out was rotated to be different for each validation.

Eight different models were built and tested (Table 3). These models included the 3D signature genes plus clinical parameters indicated. Results showed that a different model produced the optimum discrimination for each of the five subtypes tested. To assess which of the 3D genes were optimum predictors for each subtype, we performed univariate analysis. Table 4 shows that the 3D signature includes a combination of different genes that accurately predict chemotherapy response in specific breast cancer subtypes.

TABLE 4

| | Gene | | PREDICTION of Chemotherapy Response | | | | | | PROGNOSIS | |
|---|---|---|---|---|---|---|---|---|---|---|
| | Symbol | Description | ER+ | ER− | Lum A | Lum B | ERBB+ | Basal | (Kaplan p) | Functional Pathway |
| 1 | EPHA2 | EPH receptor A2 | 0.196 | 0.079 | 0.839 | 0.437 | 0.140 | 0.314 | 0.01 | angiogenesis |
| 2 | FGFBP1 | fibroblast growth factor | 0.272 | 0.060 | 0.564 | 0.055 | 0.895 | 0.087 | >0.05 | angiogenesis |
| | | binding protein 1 | | | | | | | | |
| 3 | TNFRSF6B | TNF receptor family, | 0.603 | 0.100 | 0.452 | 0.201 | 0.180 | 0.167 | >0.05 | anti-apoptosis |
| | | [symbol] b, decoy | | | | | | | | |
| 4 | FOXM1 | forkhead box M1 | 0.077 | 0.739 | 0.897 | 0.680 | 0.079 | 0.951 | 0.002 | cell cycle |
| 5 | CDKN3 | cyclin-dependent kinase | 0.678 | 0.560 | 0.199 | 0.950 | 0.523 | 0.978 | 0.002 | cell cycle: G1 progression |
| | | inhibitor 3 | | | | | | | | |
| 6 | RRM2 | ribonucleotide | 0.020 | 0.088 | 0.105 | 0.023 | 0.383 | 0.196 | 0.005 | cell cycle: G1/S |
| | | reductase M2 | | | | | | | | |
| 7 | CKS2 | CDC28 protein kinase | 0.084 | 1.000 | 0.014 | 0.773 | 0.025 | 0.635 | 0.02 | cell cycle: G2 progression |
| | | regulatory subunit 2 | | | | | | | | |
| 8 | ASPM | abnormal spindle homolog | 0.018 | 0.227 | 0.239 | 0.036 | 0.547 | 0.165 | 0.003 | cell cycle: mitotic |
| | | | | | | | | | | spindle function |
| 9 | AURKA | aurora kinase A | 0.167 | 0.939 | 0.564 | 0.899 | 0.736 | 0.480 | 0.001 | cell cycle: mitotic |
| | | | | | | | | | | spindle function |
| 10 | CEP55 | centrosomal protein 55kDa | 0.745 | 0.380 | 0.851 | 0.397 | 0.611 | 0.881 | 0.002 | cell cycle: mitotic |
| | | | | | | | | | | spindle function |
| 11 | TRIP13 | thyroid hormone receptor | 0.025 | 0.828 | 0.668 | 0.069 | 0.204 | 0.875 | 0.003 | cell cycle: mitotic |
| | | interactor 13 | | | | | | | | spindle function |
| 12 | TUBG1 | tubulin, gamma 1 | 0.178 | 0.876 | 0.017 | 0.168 | 0.201 | 0.778 | >0.05 | cell cycle: mitotic |
| | | | | | | | | | | spindle function |
| 13 | ZWILCH | Zwilch, kinetochore | 0.783 | 0.854 | 0.278 | 0.648 | 0.145 | 0.954 | >0.05 | cell cycle: mitotic |
| | | associated, homolog | | | | | | | | spindle function |
| 14 | VRK1 | vaccinia related kinase 1 | 0.527 | 0.623 | 0.537 | 0.972 | 0.119 | 0.429 | 0.001 | cell cycle: S-phase |
| | | | | | | | | | | progression |
| 15 | SERPINE2 | serpin peptidase inhibitor | 0.372 | 0.221 | 1.000 | 0.448 | 0.065 | 0.484 | >0.05 | ECM/metastasis |
| | | (nexin) 2 | | | | | | | | |
| 16 | ODC1 | ornithine decarboxylase 1 | 0.451 | 0.078 | 0.038 | 0.080 | 0.675 | 0.138 | >0.05 | polyamine biosynthesis |
| 17 | CAPRIN2 | caprin family member 2 | 0.426 | 0.517 | 0.653 | 0.870 | 0.954 | 0.312 | >0.05 | signaling pathway: WNT |
| 18 | ACTB | actin, beta | 0.437 | 0.030 | 0.558 | 0.378 | 0.019 | 0.085 | 0.007 | signaling pathways: |
| | | | | | | | | | | [symbol] -cad/b-catenin |
| 19 | ACTN1 | actinin, alpha 1 | 0.583 | 0.239 | 0.569 | 0.741 | 0.200 | 0.553 | 0.01 | signaling pathways: |
| | | | | | | | | | | [symbol] -cad/b-catenin |
| 20 | CAPG | capping protein (actin), | 0.623 | 0.906 | 0.445 | 0.309 | 0.093 | 0.618 | >0.05 | signaling pathways: |
| | | geisolin-like | | | | | | | | [symbol] -cad/b-catenin |
| 21 | DUSP4 | dual specificity | 0.896 | 0.002 | 0.570 | 0.028 | 0.012 | 0.030 | 0.0004 | signaling pathways: |
| | | phosphatase 4 | | | | | | | | EGFR and ERK |
| 22 | EIF4A1 | eukaryotic translation | 0.386 | 0.431 | 0.784 | 0.426 | 0.040 | 0.779 | >0.05 | translation |
| | | initiation factor 4A1 | | | | | | | | |

[symbol]

indicates data missing or illegible when filed

Table 4 provides a list of 3D Signature genes grouped by functional pathway with results of univariate logistic regression analysis in breast cancer subtypes. Results show that different combinations of genes discriminate chemotherapy response in each breast cancer subtype. Univariate analysis p-values are shown.

The 3D Signature provides accurate and personalized information to predict response to chemotherapy in breast cancer. In addition, the Signature predicts response in a broad range of molecular subtypes of breast cancer, including ER+, ER−, luminal A and B, basal-like and HER2+. Broad applicability of this Signature is due to a broad range of functional pathways among the signature genes. This novel approach to signature discovery is a powerful approach that can enhance the range of applicability of resulting signatures. Accurate prediction of chemotherapy response is greatly improved by including molecular class information. This gene signature has the potential to fill the existing need for an in vitro diagnostic to provide accurate and personalized information to guide chemotherapy decisions.

Combination chemotherapy regimens for breast cancer provide significant improvements in disease-free survival. Accurate stratification of patients prior to treatment may allow non-responders to receive an alternative treatment in a timely manner and potentially increase rates of complete response.

Embodiments of the present disclosure are directed to a 22-gene signature that accurately predicts response to antimitotic combination chemotherapy for breast cancer. This signature was determined based on a disruption in one of the key steps of tumorigenesis, namely disruption of the formation of spatially accurate mammary ductal units by breast epithelial cells. Hence, the 22 genes represent a biological process that is independent of any specific patient set or predefined clinical classification.

### Example 2

To determine whether genes with differential expression during human mammary acinar morphogenesis predict response to combination chemotherapy in breast cancer, results from two published microarray datasets (Fournier, et al., 2006; Popovici et al., 2010) were analyzed. Expression levels of the majority of genes that were coordinately down regulated during acini formation were significantly associated with response to combination chemotherapy treatment. A 22-gene signature representing the down regulated genes was evaluated independently in each of three breast cancer clinical subgroups, ER-positive (n=146), HER2-positive (n=41), and triple negative (n=90) using two methods of analysis, hierarchical clustering and logistic regression.

Hierarchical cluster analysis results showed that the 22 genes accurately stratified patients in each of the three subgroups by response to chemotherapy (Fisher's Exact p<0.05). Logistic regression with 3-fold cross validation demonstrated that different models accurately predicted response in these subgroups (AUC≧0.7).

Embodiments of the present disclosure demonstrate that the 22-gene signature is broadly effective across independent patient clinical subgroups in its ability to stratify patients according to chemotherapy response in breast cancer.

In one embodiment, the 22-gene signature may provide patients, early in the care process, with accurate and personalized information to predict response to combination chemotherapy.

Cluster analysis is the assignment of a set of observations into subsets (called clusters) so that observations in the same cluster are similar in some sense. It is a discovery approach generally applied to find patterns of gene expression in the absence of any prior information on the groups that one expects to find in the dataset. The method is unsupervised, meaning that it requires no pre-existing clinical information in order to separate a dataset into subgroups. Statistically, it is an approach based on correlation coefficients. In contrast to cluster analysis, logistic regression is a predictive modeling tool and a rigorous statistical approach. Logistic regression fits data to an S-shaped curve and finds the best equation (i.e. algorithm or model) to apply the expression levels of a set of genes to predict a given clinical outcome.

To predict response to chemotherapy in breast cancer, logistic regression analysis is performed by using SAS software. A model is generated based on the expression levels of the 22 genes. An “area under the curve” (AUC) is calculated and used for statistics from receiver operating curves (ROC) using three-fold cross-validation. Cross-validation, sometimes called rotation estimation, is a technique for assessing how the results of a statistical analysis will generalize to an independent data set. This method is used to estimate how accurately the predictive models will perform in practice. One round of cross-validation involves partitioning the dataset into three subsets, performing the analysis on two combined subsets (called the training set), and validating the analysis on the third subset (called the validation set or testing set). To reduce variability, three rounds of cross-validation are performed by rotating through all combination of the three subsets, and finally the validation results (AUC values) are averaged over the rounds.

The AUC value can be interpreted as the probability that the test result from a randomly chosen responsive patient is more likely to respond to chemotherapy than that from a randomly chosen nonresponsive individual. So, it can be thought of as a nonparametric distance between responsive and nonresponsive test results. AUC values are generally interpreted as follows: 0.5 to 0.6 is a poor test, 0.6 to 0.7 is a fair test, 0.7 to 0.8 is a good test, 0.8-0.9 is a very good test, and above 0.9 is an excellent test. For comparison, the AUC value for the currently marketed PSA test (prostate serum antigen) used as an early detection screen for prostate cancer is 0.57.

### Example 3

Logistic regression results for two datasets (referred to here as datasets A and B) and specific subtypes of breast cancer are presented as AUC statistics (Table 5). Both of these datasets include microarray data collected from a set of fine needle aspirate tumor biopsy samples obtained from women with breast cancer prior to neoadjuvant combination chemotherapy with TFAC (taxol, 5-fluorouracil, cyclophosphamide, and doxorubicin).

TABLE 5
The 22-gene signature accurately predicted response to chemotherapy
in two breast cancer datasets

| Dataset A | Dataset B | |
|---|---|---|
| (n = 243) | (n = 454) | Genes included in model |
| 0.701 | 0.722 | ODC1 TRIP13 DUSP4 SERPINE2 VRK1 |
| | | FGFBP1 TUBG EPHA2 |
| 0.741 | 0.763 | ODC1 TRIP13 SERPINE2 FGFBP1 TUBG |
| 0.733 | 0.726 | ODC1 TRIP13 DUSP4 SERPINE2 VRK1 |
| | | EPHA2 |
| 0.748 | 0.761 | ODC1 TRIP13 SERPINE2 TUBG |
| 0.748 | 0.774 | ODC1 TRIP13 SERPINE2 FGFBP1 |
| 0.722 | 0.742 | ODC1 TRIP13 SERPINE2 FGFBP1 DUSP4 |
| | | VRK1 |
| 0.740 | 0.761 | ODC1 TRIP13 SERPINE2 FGFBP1 DUSP4 |
| 0.758 | 0.775 | ODC1 TRIP13 SERPINE2 |
| 0.662 | 0.713 | All 22 genes |

Dataset A (n = 133), Hesss et al.
Dataset B (n = 454), Popovici et al; Tabchy et al.

Dataset A included data from 133 patients (Hess et al., 2006), while dataset B included data from an overlapping dataset of 243 patients (Popovici et al., 2010). Dataset A is a subset of the dataset B samples. For each dataset, a variety of combinations and subsets of the 22 genes were tested for predictive accuracy using logistic regression.

The first example shows results for all subtypes of breast cancer samples considered together. Results for a series of eight different subsets of the 22 genes as well as all 22 genes are listed (Table 5). AUC values range from 0.662 to 0.775. These results show that the 22-gene signature accurately predicted response to chemotherapy in both datasets.

Additional examples show logistic regression results for different subtypes of breast cancer considered independently. For example, such data demonstrates results for breast cancer molecular subtypes including ER-positive, ER-negative, luminal B and basal-like. (The luminal B subtype is a subset of ER-positive breast cancers and basal-like is subset of ER-negative breast cancers.) The latter class predominantly includes patients of the triple negative treatment group. ER status was determined by standard clinical testing. The assignment of luminal B and basal-like molecular class of tumor samples in the extended dataset of Hess et al. was performed using the intrinsic gene set of 300 genes. 263 of these genes were translated onto Affymetrix HG-U133A GeneChips and expression profiles were organized by hierarchical clustering with Pearson metric. Clusters were identified as: Luminal A=high ESR1, low AURKA; Luminal B=high ESR1, high AURKA; HER2+=high ERBB; Basal-like=low ESR1, high KRT5.

Table 6 shows results of logistic regression using expression levels of genes of the 22-gene signature to predict response to chemotherapy in 243 patients of Popovichi et al. In this example, the model (which is referred to as Model 1 or M1) was trained on all 243 patient samples and then tested on the specific subtypes listed. The model that resulted in the best results across patient subgroups is highlighted in yellow.

TABLE 6
Results of logistic regression using expression levels of genes of the 22 genes trained on the set of
all patients (M1) to predict response to chemotherapy in patients of Dataset A (Popovichi et al.).

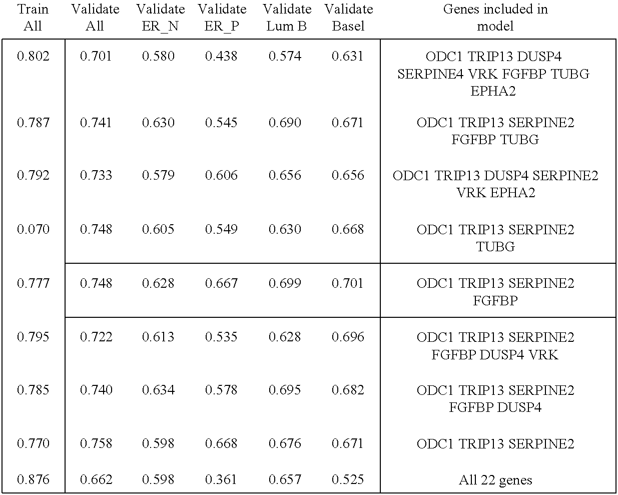

Subsequently it was tested whether adding subtype information to the 22 gene expression levels would improve response prediction (M2). To add subtype information, it was specified whether the sample was classified as ER-positive, ER-negative, luminal B or basal-like. Results showed that the inclusion of subtype information improved the prediction of response for the class of all tumors, but had no impact on any of the subclasses (Table 7). Inclusion of subtype information increased the AUC for prediction of all tumors from 0.748 (Table 6) to 0.825 (Table 7). For all other classes tested, the inclusion of subtype did not markedly increase AUC's. The model that resulted in the best results across all subtypes is highlighted in yellow.

TABLE 7
Results of logistic regression using expression levels of the 22 genes plus subtype information trained on
the set of all patients (M2) to predict response to chemotherapy in patients of dataset A (Popovichi et al.).

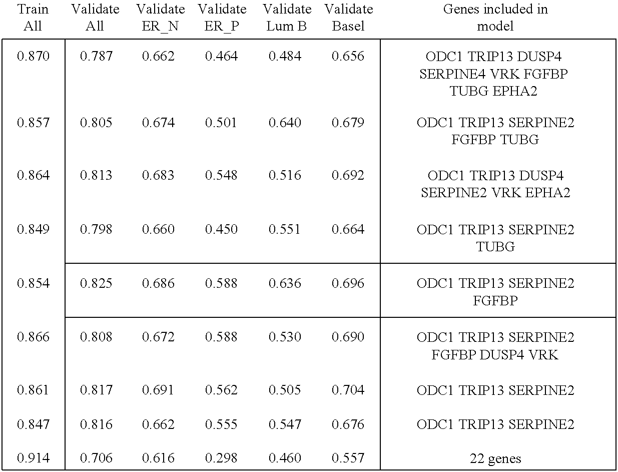

It was subsequently tested whether training the model on a specific subtype of patients would affect predictive outcome. The model M6-N was first trained on data for patients with ER-negative tumors. Results are tabulated (Table 8) and show that for each gene set tested training on ER-negative patients improved AUC in comparison to training on all patients for predictions on ER-negative patients. Surprisingly, these results showed that training on ER-negative patient's samples also improved the predictions for ER-positive patients for the gene combination of ODC1, TRIP13, SERPINE2, and FGFBP.

TABLE 8
Results of logistic regression using expression levels of the 22 genes trained on ER-
negative patients of Dataset B (M6-N) to predict response to chemotherapy in Dataset A
(Popovichi et al.).

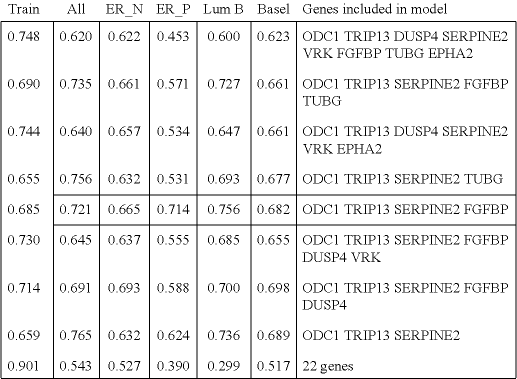

Subsequently the outcome of training the model on patients with ER-positive tumors (M6-P) was tested. Results are tabulated (Table 9) and show that for each gene set tested, training on ER-positive patients did not improve predictions in comparison to training on all patients. This unexpected result may reflect the small number of responsive patients in this breast cancer subset. The model that resulted in the best prediction results for each subtype is highlighted.

TABLE 9
Results of logistic regression using expression levels of the 22 genes trained on ER-positive patients of
Dataset B (M6-P) to predict response to chemotherapy in Dataset A (Popovichi et al.).

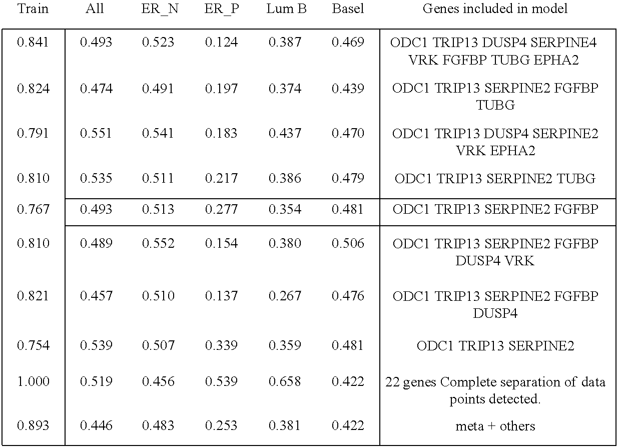

Since our results for the inclusion of subtype information improved the prediction of response for the class of all tumors, we next tested the outcome of adding expression levels of three molecular subtype classifier genes, ESR1, HER2, and CAD3 to expression levels of the 22 genes to train models (M9) was tested. The objective here was to test whether gene expression parameters could be included within the test such that externally provided parameters, such as clinical ER-status or HER2 status, would not need to be taken into account to predict chemotherapy response. The three molecular classifier genes were selected from the intrinsic gene set of Hu et al., as they represented the center genes for the major gene clusters in our cluster analysis of the TFAC dataset of Popovici et al (Dataset B). Hence these expression levels of these genes distinguish between the molecular subtypes luminal A/B, Her2+ and basal-like. Results of logistic regression are tabulated (Table 10) and show modest increases for several subsets. The model that resulted in the best AUC results for each subtype is highlighted in gray. Significantly, the additional of the three classifier genes improved performance of the 22 gene signature as well as the addition clinical subtype information. Hence addition of these genes to the 22 genes provides a method where externally provided parameters, such as clinical ER-status or HER2 status, would not need to be taken into account to predict chemotherapy response.

TABLE 10
Results of logistic regression using expression levels of the 22 genes trained on all patients of Dataset A with
expression data for 3 classifier genes added (M9) to predict response to chemotherapy in Dataset
B (Popovichi et al.).

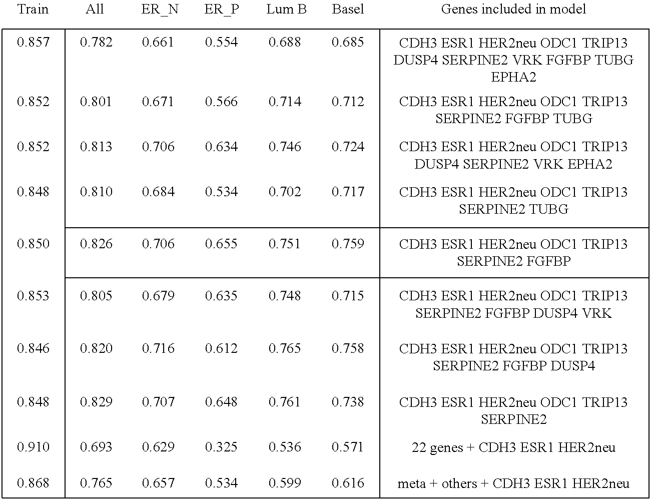

Finally the outcome of adding clinical parameters (including ER status, HER2 status, tumor size, tumor grade, patient age, patient node status, and patient race) to expression levels of 22 genes and three molecular subtype classifier genes to models to train response prediction (M10, M11, and M12) was tested. Results for all models are tabulated for comparison (Table 11). The model that resulted in the best AUC results (+/−0.02) for each subtype is highlighted.

TABLE 11
Results of logistic regression comparing the specified models to predict response to
chemotherapy in Dataset A (Popovichi et al.).

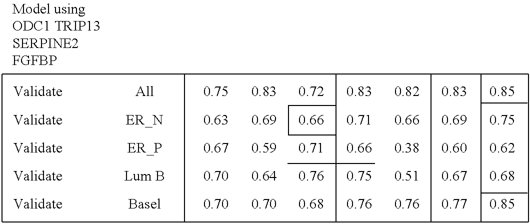

M1: 22 gene signature
M2: M1 + subtype
M6-N: M1 trained over ER negative only
M9: classifier genes CDH3, ESR1, and HER2/neu added
M10: clinical data
M11: clinical plus 22 genes plus subtype
M12: add 3 classifier genes to M11

In summary, the optimum prediction of response by the 22 signature in different subsets of patients required the application of different logistic regression models. Also, results for model 2 (M2), which tested the addition of the three molecular subtype classifier genes, ESR1, HER2, and CAD3 to the 22 gene signature, showed that these genes specifically improved response prediction when all breast cancer subtypes are considered together. These genes did not improve prediction when homogenous subtypes were considered. The addition of the three classifier genes to the 22 genes provides a method where externally provided parameters would not need to be taken into account to predict chemotherapy response. And finally, while a subset of the 22 genes including the four genes ODC1, TRIP13, SERPINE2, and FGFBP generally worked optimally for all patient subtypes and models, some specific models and subtypes performed optimally with different subsets of the 22 genes.

In one embodiment, adding classifier genes to the signature genes improved the predictive ability of the signature.

In yet another embodiment, clinical parameters may predict response well in the heterogeneous set of all patients but not in subsets, especially ER-positive and luminal B patients.

In yet another embodiment, Model M12, which included the 22 genes, clinical parameters, and three classifier genes, was highly predictive for ER-negative and basal-like tumors (0.75 and 0.85, respectively).

### Example 4

A chemotherapy response test to guide the selection of one chemotherapy regimen over another based a 22 gene signature: A critical challenge of breast cancer research is to reduce the impact of current aggressive therapies on the quality of life and to provide individualized treatment options. Invasive breast cancer affects an estimated 182,460 women annually in the United States and 1.3 million women worldwide. Embodiments of the present disclosure are directed to developing a chemotherapy response test for breast cancer patients with the ability to guide the selection of one chemotherapy regimen over another based on the prediction of a patient's responsiveness. This test is based on expression levels of a signature of 22 genes.

Key aspects of this project include the identification of a series of different algorithms or models through which the 22 gene signature can be applied to determine a patient's responsiveness to different chemotherapies (Multiple models), and the establishment of the range of chemotherapies to which each of these different algorithms can predict response (Chemotherapy specificity).

Multiple models: In the case where different tests (i.e. algorithms or models) can determine response to different chemotherapies, these tests can then be used together to identify the optimum method of treatment for a given patient. For example, if a test predicts response to Taxol, another test predicts response to Cisplatin and a third test predicts response to Anthracycline, then the application of all three of these tests together will allow the guidance of optimum treatment selection.

Embodiments of the present disclosure are directed to a novel approach that a single gene signature may be applied in multiple ways to predict different outcomes by using different algorithms or models. A 22 gene signature may accurately predict response to taxol-based combination chemotherapy in multiple breast cancer clinical subgroups, including ER-positive, ER-negative, luminal B and basal-like. It has further been shown that different models accurately predict response in the different subtypes. The optimized models for each subtype are different and neither can accurately predict response for the other subgroup.

Chemotherapy specificity: The chemotherapy specificity of a given chemotherapy response test is the full list of chemotherapy agents for which that test predicts response. If a patient is predicted to be non-responsive by one chemotherapy response test, in order to know what treatment to recommend to that patient as an alternative treatment, one needs to either have a prediction of chemotherapy responsive to a different chemotherapy or needs to define the chemotherapy specify of the response prediction test. Knowledge of the range of chemotherapies whose response is predicted by a given test will allow the recommendation of alternatives that are not included with in this group of chemotherapies. Since knowledge of the chemotherapy specificity of the test will assist in defining its clinical utility, methods to test the feasibility of applying the 22-gene signature to predict response to nontaxol cytotoxic chemotherapies are described herein. It is proposed to collect a dataset of estrogen receptor-negative (ER-negative) patients treated with platinum-based combination chemotherapy and to test the accuracy of the signature using quantitative RT-PCR (qRT-PCR). ER-negative breast cancer constitutes 40% of all breast cancer patients and there is currently no in vitro diagnostic on the market to assist in guiding chemotherapy treatment decisions for these patients.

### Example 5

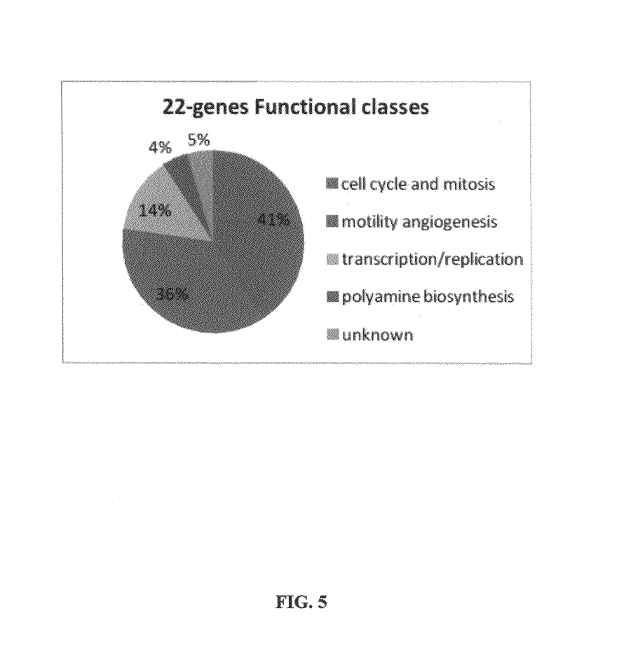

Different logistic regression models predict taxol-based chemotherapy response in different clinical subgroups: The 22-gene signature was selected in a well-defined cell culture model of nonmalignant human mammary epithelial cell morphogenesis in three dimensional laminin-rich matrix (3DlrECM) (Fournier, Martin et al. 2006). This system recapitulates key characteristics of the formation and maintenance of normal human breast ductal units (Barcellos-Hoff, Aggeler et al. 1989). Formation and maintenance of these units are disrupted in breast cancer. Genes whose expression changed during a time course of growth arrest and acquisition of basal polarity in two different isolates of human mammary epithelial cells in lrECM were identified using Affymetrix microarrays. Of 65 differentially expressed genes, 22 were down regulated and associated with breast cancer prognosis. Prognosis association was validated in 699 patients from three independent datasets (Martin, Patrick et al. 2008). This unsupervised method of signature discovery distinguishes the BIOARRAY signature from most other cancer signatures, which have been selected by supervised methods and specific patient training sets. We hypothesize that this signature has potential to more accurately classify across independent patient sets. The 22 genes signature includes functional gene classes including cell cycle, motility, and angiogenesis (FIG. 5). Identities include: EPHA2, FGFBP1, TNFRSF6B, FOXM1, CDKN3, RRM2, CKS2, ASPM, AURKA, CEP55, TRIP13, TUBG1, ZWILCH, VRK1, SERPINE2, ODC1, CAPRIN2, ACTB, ACTN1, CAPG, DUSP4, EIF4A1.

It is hypothesized that breast tumors with high expression levels of the 22 genes, which were down regulated during breast ductal units morphogenesis, were high proliferative tumors and therefore more likely to respond to antimitotics such as taxanes. To assess ability of the 22-gene signature to predict response to taxane-based chemotherapy in breast cancer, expression levels in 243 breast cancer patients treated with neoadjuvant taxane-based chemotherapy were studied in a published microarray dataset (Hess, Anderson et al. 2006). This dataset was assembled at MD Anderson Breast Cancer Center from fine-needle aspirates obtained from patients with stage I-III breast cancer. Biopsies obtained before chemotherapy with paclitaxol (most patients received an anthracycline combination regimen FAC or FEC in addition to taxol) were assessed for pathological complete response (pCR) after surgery. We assigned breast cancer subtypes by hierarchical clustering using published genes (Perou, Sorlie et al. 2000; Hu, Fan et al. 2006; Parker, Mullins et al. 2009). Clusters were identified as Luminal A=high ESR1, low AURKA; Luminal B=high ESR1, high AURKA; Her2−positive=high HER2; Basal-like=low ESR1, high KRT5.

To predict the probability of response to chemotherapy, logistic regression was applied, a robust approach that fits data to an S shaped curve. Analyses performed using SAS software generated models based on expression levels of the 22 genes using three-fold cross-validation. Results for all datasets and specific subtypes of breast cancer are presented as area under the curve (AUC) statistics (Table 6). Statistically significant results show that the 22-gene signature accurately predicted response to chemotherapy in all breast cancer subtypes tested. The 22 gene signature is a particularly good predictor of response in the subclasses of ER-negative (0.75) and triple negative (0.85) breast cancer. Prediction among ER-negative breast cancers has previously been described as a challenge; even among classifiers specifically selected from the same dataset used here, validation AUCs for ER-negative cancers only ranged from 0.34 to 0.62 (Popovici, Chen et al, 2010).

In addition to studying the 22 gene signature as a set, univariate analysis was also performed. The ability of individual genes to discriminate responders and non responders in different subtypes of breast cancer was assessed. Results showed interesting differences. Signature genes that function to regulate cell cycle and cell proliferation were generally significant discriminators of response in ER-positive cancers, while signature genes that involved in signal transduction were generally significant discriminators of response in ER-negative cancers.

### Example 6

Results showing different logistic regression models applied to the 22 gene: Results presented herein demonstrate that different logistic regression models can be applied to the 22 gene signature to accurately predict taxol-based chemotherapy response in different clinical subgroups. It is a novel finding that a single gene signature can be applied in multiple ways to predict different outcomes.

It is shown that the 22 gene signature can accurately predict response to taxol-based combination chemotherapy in multiple breast cancer clinical subgroups, including ER-positive, ER-negative, luminal B and basal-like. A series of 12 different logistic regression models using the 22 gene signature are developed and tested for their ability to predict response to chemotherapy in a series of breast cancer subtypes. These results are summarized (Table 11).

For the subtype of ER-negative breast cancers, model M12 was most accurate. This model was trained over all samples using expression levels of the 22 genes plus clinical data plus expression levels of three classifier genes.

For the subtype of ER-positive breast cancers, model M6-N was most accurate. This model was trained over ER-negative breast cancer samples and using expression levels of the 22 genes.

For the subtype of luminal B breast cancers, models M6-N and M9 were most accurate. Model M6-N was trained over ER-negative breast cancer samples and using expression levels of the 22 genes. Model M9 was trained over all samples using expression levels of the 22 genes plus expression levels of three classifier genes.

For the subtype of basal-like breast cancers, model M12 was most accurate. This model was trained over all samples using expression levels of the 22 genes plus clinical data plus expression levels of three classifier genes.

For the combined set of breast cancers from all subclasses, several models showed similar accuracy, including M2, M9, M10, M11 and M12.

Hence, the optimized models for each subtype tend to be different and do not accurately predict response for other subgroups.

### Example 7

Chemo specificity of the 22 gene response prediction signature: The example studies the ability of the 22-gene signature to predict response to platinum-based combination chemotherapy for ER-negative breast cancer by using microfluidic quantitative RT-PCR. The criterion for positive outcome is an assay that significantly outperforms clinical parameters in terms of AUC, sensitivity, and specificity (ROC analysis; p<0.05). This example includes the following steps:

Obtain 50 biopsy samples: These are retrospective, formalin-fixed, paraffin-embedded tissue biopsies obtained before any treatment from ER-negative breast cancer patients in a neoadjuvant treatment setting. Patients will have been treated with platinum-based combination chemotherapy. All samples are annotated with information of pathological complete response information and clinical parameters. Expression levels of the 22-genes in the 50 samples are measured using microfluidic qRT-PCR. The results are analyzed using logistic regression and ROC curves to determine the ability of the signature to predict response to platinum-based combination chemotherapy treatment using pathological complete response as the end point. The method is used to predict respond to platinum-based combination chemotherapy treatment using pathological complete response as the end point.

The 22-gene signature is used to accurately predict response to non-taxol chemotherapy in ER-negative breast cancer patients. For these patients, systemic chemotherapy improves the odds of disease-free and overall survival whereas hormonal therapy is not helpful. For the subgroup of Her2-positive patients, therapies that target Her2 are highly effective. But for triple negative cancers, (ER-negative, PR-negative, Her2-negative), which lack a target for therapy, systemic chemotherapy with a standard cytotoxic agent is the single major treatment option (Schneider, Winer et al. 2008). Ongoing clinical trials indicate that new therapies that target PARP, src, EGFR and VEGF may add more options for ER-negative patients in the future (Carey, Winer et al. 2010; Silver, Richardson et al. 2010). Since studies have found that patients with triple-negative cancers experience shorter disease-free and overall survival times than patients with other types of breast cancer, guiding effective treatment options is highly important. Neoadjuvant studies indicate ER-negative tumors respond well to anthracycline-based or anthracycline and taxane-based chemotherapy. Other agents studied include DNA-damaging agents (i.e. platinum compounds), because a large percentage of ER-negative patients carry germ line mutations in BRCA1, which plays an important role in DNA-damage repair. These compounds include cisplatin, carboplatin and irinitecan. While ER-negative tumors have been found to have a higher likelihood of response to cytotoxic chemotherapy than ER-positive tumors, a complete response to chemotherapy is more important in this group where there is no targeted therapy available. Patients must experience a pathological complete response (pCR) to chemotherapy with no residual tumor cells remaining for a long relapse free survival (Rouzier, Perou et al. 2005). For women with ER-negative cancer, strategies to maximize chemotherapy effectiveness have the potential to reduce relapse and mortality, and, by avoiding ineffective treatments, to increase quality of life and reduce health care costs. The predicted response is determined based upon a multivariate gene expression signature that accurately predicts response to chemotherapy in ER-negative breast cancer.

### Example 8

### Prediction of Taxol Combination (TFAC) Versus Non-Taxol Combination (FAC)

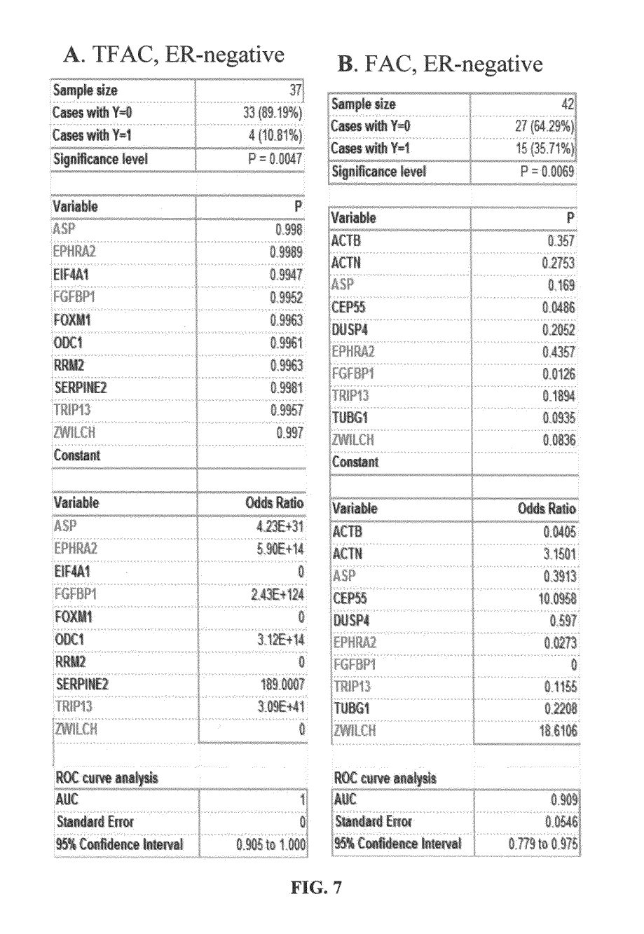

A comparison logistic regression output results was performed by using MedCalc software to assess the ability of the 22 gene signature to predict response to taxol combination (TFAC) versus non-taxol combination (FAC) chemotherapy response in breast cancer using logistic regression. This study used a simplified version of logistic regression, where AUCs were calculated on the training set and no test sets or cross validation is applied. The objective of this experiment was to test if the 22 gene model that predicts TFAC response also predicts FAC response. Microarray data from a randomized trial with two arms, TFAC and FAC, were collected at MD Anderson Cancer Center (Tabchy et al 2010). The gene signature was optimized by sequentially omitting from the analysis genes with lowest p values. Discovery logistic regression results from 37 ER-negative samples from patients treated with TFAC are shown (FIG. 7, panel A). Resulting perfect AUC of 1.00 indicates a ideal prediction test that is statistically significant (p<0.0047). Discovery logistic regression results from 42 ER-negative samples from patients treated with FAC are shown (FIG. 7, panel B). The resulting AUC of 0.909 indicates an excellent test that is statistically significant (p=0.0069). The results indicate that expression levels of the 22 genes allow accurate prediction of response to both TFAC and FAC. Interestingly; however, the optimized models differ markedly. Only 50% of optimized genes are overlapping and for these overlapping genes, odds ratio vary greatly between the two datasets. Hence, it is concluded that the 22 gene signature has the potential to accurately predict response to both taxol combination chemotherapy and non taxol combination chemotherapy by using logistic regression different models.

### Example 9

### Prediction of Taxol Combination (TFAC) Versus Cisplatin

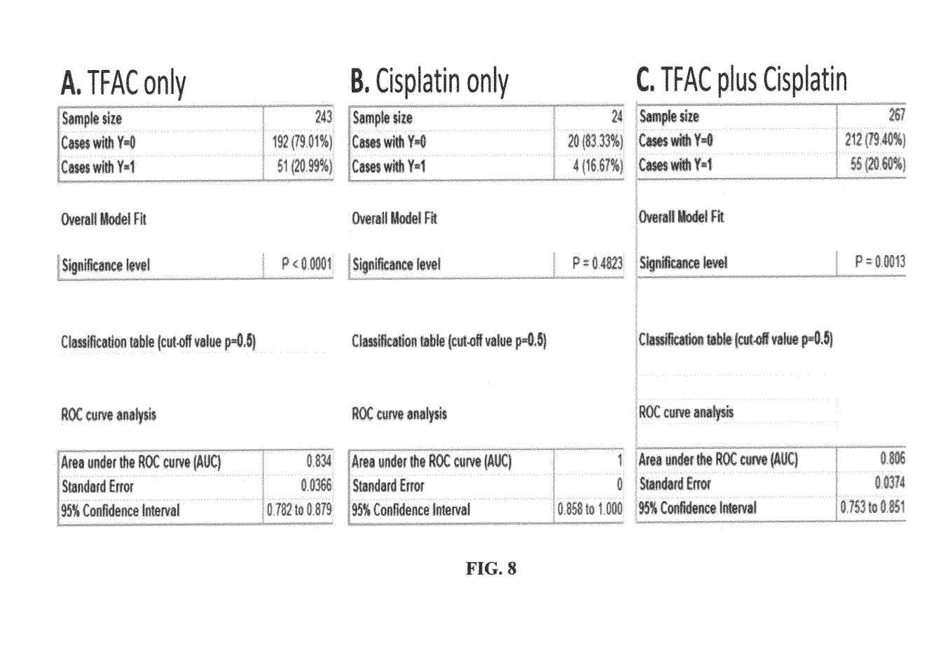

We have compared the ability of the 22 gene signature to predict response to taxol combination is compared to a single agent cisplatin chemotherapy response in breast cancer using logistic regression. This study used a simplified version of logistic regression, where AUCs are calculated on the training set and no test sets or cross validation is applied. The objective of this experiment was to test if the same 22 gene model that that predicts TFAC response also predicts cisplatin response. Microarray data for the 24 biopsy samples from patients subsequently treated with neoadjuvant cisplatin were collected at the Dana Farber Cancer Institute (Silver et al 2010). Discovery logistic regression results from 243 samples from patients treated with TFAC (Popovici et al 2010) are shown (FIG. 8, panel A). The resulting AUC of 0.834 indicates a very good prediction test that is statistically significant (p<0.0001). Discovery logistic regression results from 24 samples from patients treated with cisplatin (Silver et al 2010) are shown (FIG. 8, panel B). The resulting AUC of 1.0 indicates a perfect test, though the number of samples was to low to achieve statistical significance (p=0.4823). Discovery logistic regression analysis of the combined datasets of TFAC and cisplatin was performed to test whether the same model was applicable to both datasets. An AUC of 0.806 was obtained (FIG. 8, panel C), which is less than the results of 0.834 obtained for the TFAC dataset alone, though it is not outside of the 95% confidence limits. In summary, though samples numbers were not large enough to obtain significance, these results appear to suggest that expression levels of the 22 genes allowed the prediction of response to both cisplatin and TFAC. Importantly, these predictions appeared to use different models. Hence, if a patient were responsive to one chemotherapy treatment but nonresponsive to the other, it appears that the 22 genes could potentially distinguish between these options and identify the better treatment for the patient.

### Example 10

### Methods

22-gene signature is evaluated to predict response to cytotoxic chemotherapies for breast cancer using microfluidic quantitative RT-PCR. The criterion for acceptance is an assay that significantly outperforms clinical parameters in terms of AUC, sensitivity, and specificity (ROC analysis; p<0.05). Approximately 50 biopsy samples are obtained. The samples are retrospective, formalin-fixed, paraffin-embedded tissue biopsies obtained before treatment of ER-negative breast cancer patients in a neoadjuvant treatment setting. Patients will have been treated with a platinum-based combination chemotherapy regimen. All samples are annotated with response information and data on clinical parameters.

Expression levels of the 22-genes in the 50 samples are measured using microfluidic qRT-PCR. RT-PCR results are analyzed using logistic regression and ROC curves to determine ability of the signature to predict response to platinum-based chemotherapy using pCR as an end point. using qRT-PCR shows that the 22-gene signature accurately predicts response to platinum-based combination chemotherapy for ER-negative breast cancer patients.

Breast cancer biopsies are analyzed by microfluidic quantitative RT-PCR using validated probes and primers. Reverse transcription and PCR reactions are performed as recommended. Logistic regression is used to predict the probability of response. Analyses is performed using SAS software and results presented as AUC statistics. Microfluidic RT-PCR. RT-PCR is the most sensitive technique for mRNA detection and quantification currently available. It is a robust sensitive tool used for routine clinical diagnostics. It is faster, cheaper, and more sensitive than cDNA microarrays. RT-PCR is often used to validate microarray results. Concordance of the microarray with RT-PCR results has been reported to be high (Espinosa, Sanchez-Navarro et al. 2009). Applied Biosystems (Foster City, Calif.) TaqMan Low-Density Arrays (TLDA) is a medium-throughput method for real-time RT-PCR that uses micro fluidics. TLDA cards allow simultaneous measurement of RNA expression for up to 384 genes per card. Wells are custom prepared to include forward and reverse primers (900 nM concentrations) and TaqMan MGB probe (6-FAM dye-labeled, 250 nM). Assays use TLDA cards designed to include probes for each of the 22 genes, 8-10 control reference genes, 4 replicates per gene (standard replicate level for TLDA cards), in 384-well format. Standard, commercial primers are used. Reference controls include tyrosine 3/tryptophan 5-monooxygenase activation protein (YMHAZ), TATAA-box binding protein (TBP), beta-glucuronidase (GUSB) and additional genes. The delta [Ct] method is used to quantify gene expression levels. Inclusion of multiple reference genes (5-10 genes) helps to assure that the mean reference value is consistent across all samples. Relative copy number for two samples (experimental and control) is determined by the difference between Ct values. Relative gene expression quantities (delta delta [Ct] values) are obtained by normalization against reference genes. Non-responding control patients are integral to the dataset. TLDA cards are used and micro fluidic qRT-PCR is performed. Cards are initially evaluated with control samples. Cell line RNAs obtained from the ATCC are used as controls to standardize results over time. All samples are run in triplicate.

Perform RT-PCR of 50 ER-Negative Breast Cancer Samples

Core biopsies are collected from women age 70 or younger with ER-negative stage I-III breast cancer, independent of lymph node status. Biopsy samples are collected before starting preoperative chemotherapy with a platinum-based combination chemotherapy regimen. All patients will sign an informed consent for voluntary participation. Samples are selected without regard to outcome. Pathological complete response (pCR) is used as the study end point and is defined as no residual invasive cancer in breast or lymph nodes as assessed by pathology evaluation. Residual in situ carcinoma without an invasive component is considered a pCR.

Yields of greater than 100 ug total RNA are required for microfluidic RT-PCR. Previous studies report yields of at least 1 g from most tumor samples (Hess, Anderson et al. 2006). Samples are assessed by a pathologist to determine percent tumor and only those containing at least 50% neoplastic cells are included in the study. RNA is purified by standard methods. Total RNA is extracted by RNAeasy Mini Kit (Qiagen, Hilden, Germany) and quality checked by Bioanalyzer 2100 (Agilent Technologies, Palo Alto, Calif.).

A priori power analysis allows calculation of sample size required for a two group study. Power analysis based on expression levels and response prediction by the 22 genes in the microarray dataset of Chang, et al. (Chang, Wooten et al. 2003) indicates a requirement for a minimum of 49 samples for significance at the 95% confidence level. Though this study included patients who had received docetaxel chemotherapy (data not shown), it is hypothesized similar sample variability will apply to response prediction in a cross set of non-taxane treated patients. Hence, this example uses 50 samples. Samples are purchased through Analytical Biosciences Inc. (ABS). All samples will have complete annotated clinical information including chemotherapy response. All information is compliant with Health Information Privacy Act of 1999 (HIPA).

Statistical tests are applied to the RT-PCR determined expression levels of the 22 genes and control genes. Performance of the assay is evaluated by ROC analysis and logistic regression using a model that will be defined from a subset of 80% of patients (training set; 40 patients). AUC's are determined by a standard 5-fold cross validation of the remaining 20% of samples (test set; 10 samples) where the hold out is rotated to be different for each validation. The AUC will reflect the quality of the assay and a minimum value of 0.60 and a p-value of <0.05 will be required.

### Example 11

### Microarray Datasets

This study used at total of five microarray datasets from a total of 610 patients. Gene discovery: A time course of acini formation in 3D culture was used for discovery of the 22 genes (Fournier, et al., 2006 Cancer Res, 66:7095). Microarrays were Affymetrix HG-U133A and have been publicly archived at GEO GSE8096. Evaluation of response prediction: Three overlapping datasets were used to evaluate the ability of the signature to predict chemotherapy response. All were obtained at MD Anderson Medical Center from fine-needle tumor aspirates from patients with stage I-III breast cancer obtained before neoadjuvant combination treatment with paclitaxel, 5-fluorouracil, cyclophosphamide and doxorubicin (TFAC) followed by surgical resection.

Response was categorized as pathological complete response (pCR, i.e. no residual invasive cancer in breast or nodes) or residual disease (RD). Microarrays were Affymetrix HG-U133A. The dataset of Hess, et al., 2006 J Clin Oncol, 24:4236 included 133 patients, while datasets of Popovici, et al., 2010 Breast Cancer Res 12:R5 included 243 patients (GEO GSE20194) and Tabchy, et al., 2010, Clin Cancer Res 16: 5351-5361 included 79 patients (GEO GSE20271). Evaluation of prognosis: Prognosis evaluation used a dataset of 286 lymph node negative patients with 5 year relapse as an endpoint (Wang et al., 2005, Lancet 365:671-679) (GEO GSE2034). Molecular classes for tumors in dataset of Popovici 2010, were determined using the intrinsic gene set of 300 genes (Hu, et al., 2006). Expression values were organized by hierarchical clustering with Pearson metric. Clusters were identified as: Luminal A=high ESR1, low AURKA; Luminal B=high ESR1, high AURKA; HER2+=high ERBB; Basal-like=low ESR1, high KRT5.

Results: Gene sets down-regulated during acini formation are enriched in genes associated with response to TFAC chemo. Gene sets were selected that were differentially regulated during a time course of morphogenesis of non-malignant breast epithelial cells in laminin-rich 3-dimensional culture. These gene sets are tabulated below and include down regulated early, down regulated late, up regulated early, up regulated late, down regulated, up regulated, early, late, all differentials and all genome. Data for 840 random lists of 22 genes are also tabulated. The total number of genes (n) in each set are listed. Also listed are the number of genes in each set that were significantly associated with response to TFAC chemotherapy using pathological complete response (pCR) as an endpoint. The set with the highest proportion of response associated genes is the down late gene set for which 55% of genes were associated with response (t-test<0.05). For 840 random gene sets of 22 genes each, an average of only 17% of genes were significantly associated with response. Hence, the gene sets down regulated during morphogenesis of breast epithelial cells in 3D culture were significantly enriched in chemotherapy response associated genes. The results are shown in the following table.

| | | | | Ability to | |
|---|---|---|---|---|---|
| Temporal | Total | Genes significantly* | | stratify by response** | |
| expression | genes | associated with pCR | | (Chi² | |
| pattern | (N) | (N) | (%) | coefficient) | (p-value) |
| Down early | 6 | 3 | 50% | 0.248 | 0.0005 |
| Down late | 22 | 12 | 55% | 0.364 | <0.000001 |
| Up early | 21 | | 5% | — | — |
| Up late | 11 | 2 | 18% | — | — |
| Down | 28 | 15 | 54% | 0.241 | 0.00059 |
| Up | 32 | 3 | 9% | — | — |
| Early | 27 | 6 | 22% | — | — |
| Late | 33 | 14 | 42% | 0.344 | <0.000001 |
| All | 60 | 22 | 37% | 0.283 | <0.000001 |
| differentials | | | | | |
| All genome | 22282 | 3766 | 17% | — | — |
| 840 random | 22 | 3.73 | 17% | — | — |
| lists | | (max 6, min 0) | | | |

*t-Test, p < 0.05, was used to evaluate genes associated with response (pCR) in the TFAC response microarray dataset of Popovici et al. 2010 (243 patients);
**Hierarchical clustering was used to stratify patients from the TFAC response microarray dataset of Hess et al. 2006 (133 patients). Chi2 coefficient and Fisher's Exact p-values are tabulated.

22-gene signature stratified breast cancer subtypes by response to TFAC chemotherapy and outperformed clinical parameters. For six breast cancer subtypes, logistic regression was used to assess the ability of the 22 gene signature to predict response to TFAC chemotherapy. AUC values are listed below. Comparison values are listed for five clinical parameters. For each subtype, the 22 gene signature outperformed all clinical parameters.

| | | AUC Value* (n) | | | | |
|---|---|---|---|---|---|---|
| Breast Cancer | | Node | ER | Tumor | Tumor | |
| Subtype | 22-genes | status | status | size | grade | Ki67 |
| ER Positive | 0.723 (208) | 0.490 | — | 0.475 | 0.689 | 0.650 |
| ER Negative | 0.744 (145) | 0.481 | — | 0.525 | 0.689 | 0.635 |
| HER2 Positive | 0.772 (42) | 0.513 | — | 0.525 | 0.316 | 0.350 |
| Triple Negative | 0.718 (95) | 0.490 | — | 0.525 | 0.689 | 0.650 |
| ER, PR, HER2 | | | | | | |
| negative) | | | | | | |
| Luminal B | 0.75 (50) | — | — | — | — | — |
| Basal-like | 0.85 (69) | — | — | — | — | — |
| All subtypes | 0.830 (353) | 0.478 | 0.760 | 0.525 | 0.689 | 0.650 |

*AUC values for 22-gene signature test and clinical parameters were determined by logistic regression with 3-fold cross validation using the datasets of Popovici et al. 2010 and Tabchy et al. 2010.

1. A method of treating breast cancer comprising
experimentally obtaining a dataset associated with a sample derived from a patient diagnosed with cancer, wherein the dataset comprises:
expression data for at least one marker selected from the group consisting of FLJ10517, HCAP-G, CDKN3, STK6, FOXM1, FLJ10540, TNFRSF6B, HBP17, C1QDC1, TUBG1, FLJ10036, RRM2, ACTB, ACTN1, EPHA2, TRIP13, CKS2, VRK1, DUSP4, EIF4A1, SERPINE2, and ODC1 and/optionally at least one clinical factor;
determining a predictive score from the dataset using an interpretation function, wherein
the predictive score is predictive of the response to the cancer treatment; and
administering a therapeutically effective amount of the cancer treatment to the patient who is predicted to respond to the cancer treatment.

2-7. (canceled)

8. The method of claim 1, wherein the determining is determined by a computer processor.

9. The method of claim 1, wherein the dataset further comprises the expression data and the at least one clinical factor.

10. The method of claim 9, wherein the at least one clinical factor term is selected from the group consisting of age, gender, neutrophil count, ethnicity, race, disease duration, diastolic blood pressure, systolic blood pressure, a family history parameter, a medical history parameter, a medical symptom parameter, height, weight, a body-mass index, smoker/non-smoker status, ER status, HER2 status, tumor size, tumor grade, luminal A characterization, luminal B characterization, basal-like, and normal-like.

11. The method of claim 1, wherein the predictive score is compared to a score derived from a sample from a patient with cancer that was known to have responded or not responded to chemotherapy,
wherein a sample whose score matches the predetermined predictive of sample derived from a patient that responded to treatment the patient diagnosed with cancer is predicted to respond to the cancer treatment, or
wherein a sample whose score matches the predetermined predictive of sample derived from a patient that did not respond to treatment the patient diagnosed with cancer is predicted to not to respond to the cancer treatment.

12. (canceled)

13. The method of claim 1, wherein said response is a complete response, partial response no response, a pathological complete response, at least 5 year survival, or a relapse-free survival.

14-16. (canceled)

17. The method of claim 1, wherein the interpretation function is based upon a predictive model.

18. The method of claim 17, wherein the predictive model is a logistical regression model, wherein the logistic regression model is applied to the dataset to interpret the dataset to produce the predictive score, wherein a predictive score above a specified cut-off value predicts responsiveness and a predictive score below a specified cut-off predicts non-responsiveness.

19. (canceled)

20. The method of claim 19, wherein the specified cut-off is selected from the group consisting of 0.1, 0.2, 0.3, 0.4, 0.5, 0.6, 0.7, 0.8, and 0.9.

21.-23. (canceled)

24. The method of claim 1, wherein the patient diagnosed with breast cancer has an ER-positive breast cancer, ER-negative breast cancer, a breast cancer characterized as Luminal B, a breast cancer characterized as basal-like, or a triple-negative breast cancer.

25-28. (canceled)

29. The method of claim 1, wherein the cancer treatment is adjuvant chemotherapy and/or neoadjuvant chemotherapy.

30. The method of claim 1, wherein the cancer treatment is a treatment selected from the group consisting of: TFAC (combination of taxol/fluorouracil/anthracycline/cyclophosphamide) TAC (taxol/anthracycline/cyclophosphamide with or without filgrastim support), ACMF (doxorubicin followed by cyclophosphamide, methotrexate, fluorouracil), ACT (doxorubicin, cyclophosphamide followed by taxol or docetaxel), A-T-C (doxorubicin followed by paclitaxel followed by cyclophosphamide), CAF/FAC (fluorouracil/doxorubicin/cyclophosphamide), CEF (cyclophosphamide/epirubicin/fluorouracil), AC (doxorubicin/cyclophosphamide), EC (epirubicin/cyclophosphamide), AT (doxorubicin/docetaxel or doxorubicin/taxol), CMF (cyclophosphamide/methotrexate/fluorouracil), cyclophosphamide (Cytoxan or Neosar), methotrexate, fluorouracil (5-FU), doxorubicin (Adriamycin), epirubicin (Ellence), gemcitabine, taxol (Paclitaxel), GT (gemcitabine/taxol), taxotere (Docetaxel), vinorelbine (Navelbine), capecitabine (Xeloda), platinum drugs (Cisplatin, Carboplatin), etoposide, and vinblastine.

31-36. (canceled)

37. The method of claim 1, the method further comprising extracting RNA from breast epithelial cells.

38. The method of claim 1, the method further comprising hybridizing the sample with one or more probes to produce the expression data.

39. The method of claim 1, the method further comprising performing polymerase chain reaction to produce the expression.

40. (canceled)

41. A system for predicting a response to a cancer treatment comprising a storage memory for storing a dataset associated with a sample obtained from the subject, wherein the dataset comprises expression data for at least one marker selected from the group consisting of FLJ10517, HCAP-G, CDKN3, STK6, FOXM1, FLJ10540, TNFRSF6B, HBP17, C1QDC1, TUBG1, FLJ10036, RRM2, ACTB, ACTN1, EPHA2, TRIP13, CKS2, VRK1, DUSP4, EIF4A1, SERPINE2, and ODC1; and a processor communicatively coupled to the storage memory for determining a score with an interpretation function wherein the score is predictive of response to a cancer treatment in a subject diagnosed with cancer.

42. (canceled)

43. The system of claim 41, wherein the cancer is breast cancer.

44. A kit for predicting response to a cancer treatment in a subject comprising one or more reagents for determining from a sample obtained from a subject expression data for at least one marker selected from the group consisting of FLJ10517, HCAP-G, CDKN3, STK6, FOXM1, FLJ10540, TNFRSF6B, HBP17, C1QDC1, TUBG1, FLJ10036, RRM2, ACTB, ACTN1, EPHA2, TRIP13, CKS2, VRK1, DUSP4, EIF4A1, SERPINE2, and ODC1; and instructions for using the one or more reagents to determine expression data from the sample, wherein the instructions include instructions for determining a score from the dataset wherein the score is predictive of response to the cancer treatment.

45-46. (canceled)

47. The kit of claim 44, wherein the cancer treatment is a breast cancer treatment.

48. The kit of claim 44, wherein the cancer treatment comprises a nitrogen mustard, a vinca alkaloid, an epothilones, a taxane, a mitotic inhibitor, a corticosteroid, a topoisomerase II inhibitor, a topoisomerase I inhibitor, an anti-tumor antibiotics, an anthracycline, an antimetabolite, an ethylenimine, an alkyl sulfonate, a nitrosourea, or any combination thereof.

49-50. (canceled)

51. A method for predicting a response to a cancer treatment in a patient diagnosed with cancer comprising:
isolating a sample of the cancer from the patient diagnosed with cancer;
obtaining a dataset associated with a sample derived from a patient diagnosed with cancer, wherein the dataset comprises expression data for at least one marker selected from the group consisting of FLJ10517, HCAP-G, CDKN3, STK6, FOXM1, FLJ10540, TNFRSF6B, HBP17, C1QDC1, TUBG1, FLJ10036, RRM2, ACTB, ACTN1, EPHA2, TRIP13, CKS2, VRK1, DUSP4, EIF4A1, SERPINE2, and ODC1 and at least one clinical factor; and
determining a predictive score from the dataset using an interpretation function,
wherein the interpretation function comprises is based upon a predictive model,
wherein the predictive model is a logistical regression model,
wherein the logistical regression model is applied to the dataset to interpret the dataset to produce the predictive score, and
wherein a predictive score above a specified cut-off value predicts responsiveness and a predictive score below a specified cut-off predicts non-responsiveness.


SEQUENCE LISTING (32 sequences)

SEQ ID NO: 1 - DNA, 555 bp, homo sapiens
tatgacttagttgcgttacaccctttcttgacaaaacctaacttgcgcagaaaacaagatgagattggcatggctttatttgttttttttgttttgttttggttttttttttttttttggcttgactcaggatttaaaaactggaacggtgaaggtgacagcagtcggttggagcgagcatcccccaaagttcacaatgtggccgaggactttgattgcacattgttgtttttttaatagtcattccaaatatgagatgcattgttacaggaagtcccttgccatcctaaaagccaccccacttctctctaaggagaatggcccagtcctctcccaagtccacacaggggaggtgatagcattgctttcgtgtaaattatgtaatgcaaaatttttttaatcttcgccttaatacttttttattttgttttattttgaatgatgagccttcgtgcccccccttccccctttttgtcccccaacttgagatgtatgaaggcttttggtctccctgggagtgggtggaggcagccagggcttacctgtacactgact

SEQ ID NO: 2 - DNA, 25 bp, artificial sequence
  primer
tatgacttagttgcgttacaccctt

SEQ ID NO: 3 - DNA, 25 bp, artificial sequence
  primer
cagcagtcggttggagcgagcatcc

SEQ ID NO: 4 - DNA, 25 bp, artificial sequence
  primer
gcatcccccaaagttcacaatgtgg

SEQ ID NO: 5 - DNA, 25 bp, artificial sequence
  primer
ggccgaggactttgattgcacattg

SEQ ID NO: 6 - DNA, 25 bp, artificial sequence
  primer
ttgttacaggaagtcccttgccatc

SEQ ID NO: 7 - DNA, 25 bp, artificial sequence
  primer
taaggagaatggcccagtcctctcc

SEQ ID NO: 8 - DNA, 25 bp, artificial sequence
  primer
ttttgaatgatgagccttcgtgccc

SEQ ID NO: 9 - DNA, 25 bp, artificial sequence
  primer
tttttgtcccccaacttgagatgta

SEQ ID NO: 10 - DNA, 25 bp, artificial sequence
  primer
tgtatgaaggcttttggtctccctg

SEQ ID NO: 11 - DNA, 25 bp, artificial sequence
  primer
ggagtgggtggaggcagccagggct

SEQ ID NO: 12 - DNA, 25 bp, artificial sequence
  primer
gccagggcttacctgtacactgact

SEQ ID NO: 13 - DNA, 543 bp, homo sapiens
ggtcccgaggagttcaaagcctgcctcatcagcttgggttatgatattggcaacgacccccagggagaagcagaatttgcccgcatcatgagcattgtggaccccaaccgcctgggggtagtgacattccaggccttcattgacttcatgtcccgcgagacagccgacacagatacagcagaccaagtcatggcttccttcaagatcctggctggggacaagaactacattaccatggacgagctgcgccgcgagctgccacccgaccaggctgagtactgcatcgcgcggatggccccctacaccggccccgactccgtgccaggtgctctggactacatgtccttctccacggcgctgtacggcgagagtgacctctaatccaccccgcccggccgccctcgtcttgtgcgccgtgccctgccttgcacctccgccgtcgcccatctcctgcctgggttcggtttcagctcccagcctccacccgggtgagctggggcccacgtggcatcgatcctccctgcccgcgaagtgacagtttac

SEQ ID NO: 14 - DNA, 516 bp, homosapiens
gttgtaatcgcagtattccttgtatggaagtcatcagatatgctgtgcaagtcttgcttaatgtatctaagtatgagaaaactacttcagcagtttatgatgtagaaaattgtatagatatactattggagcttttgcagatataccgagaaaagcctggtaataaagttgcagacaaaggcggaagcatttttacaaaaacttgttgtttgttggctattttactgaagacaacaaatagagcctctgatgtacgaagtaggtccaaagttgttgaccgtatttacagtctctacaaacttacagctcataaacataaaatgaatactgaaagaatactttacaagcaaaagaagaattcttctataagcattccttttatcccagaaacacctgtaaggaccagaatagtttcaagacttaagccagattgggttttgagaagagataacatggaagaaatcacaaatcccctgcaagctattcaaatggtgatggatacgcttggcattcctt

SEQ ID NO: 15 - DNA, 520 bp, homo sapiens
aaggatcttaactgtgttcgcattttttatccaagcacttagaaaacctacaatcctaattttgatgtccattgttaagaggtggtgatagatactatttttttttcatattgtatagcggttattagaaaagttggggattttcttgatctttattgctgcttaccattgaaacttaacccagctgtgttccccaactctgttctgcgcacgaaacagtatctgtttgaggcataatcttaagtggccacacacaatgttttctcttatgttatctggcagtaactgtaacttgaattacattagcacattctgcttagctaaaattgttaaaataaactttaataaacccatgtagccctctcatttgattgacagtattttagttatttttggcattcttaaagctgggcaatgtaatgatcagatctttgtttgtctgaacaggtatttttatacatgctttttgtaaaccaaaaacttttaaatttcttcaggttttctaacatgcttaccactg

SEQ ID NO: 16 - DNA, 549 bp, homo sapiens
gaatgtgccactgtatgtcaacctcatgaagaatgaagaggtcttggtatcagcctatgccaatgatggtgctccagaccatgaaactgctagcaatcatgcaattcttcagctcttccagggagaccagatatggttacgtctgcacaggggagcaatttatggaagtagctggaaatattctacgttttcaggctatcttctttatcaagattgaaagtcagtacagtattgacaataaaaggatggtgttctaattagtgggattgaaggaaaagtagtctttgccctcatgactgattggtttaggaaaatgtttttgttcctagagggaggaggtccttacttttttgttttccttcctgaggtgaaaaatcaagctgaatgacaattagcactaatctggcactttataaattgtgatgtagcctcgctagtcaagctgtgaatgtatattgtttgcacttaatccttaactgtattaacgttcagcttactaaactgactgcctcaagtccaggcaagttacaatgccttgttgtgcctcaa

SEQ ID NO: 17 - DNA, 426 bp, homo sapiens
aaagatacagaagaactaaagagctgtggtatacaagacatatttgttttctacaccagaggggaactgtcaaaatatagagtcccaaaccttctggatctctaccagcaatgtggaattatcacccatcatcatccaatcgcagatggagggactcctgacatagccagctgctgtgaaataatggaagagcttacaacctgccttaaaaattaccgaaaaaccttaatacactgctatggaggacttgggagatcttgtcttgtagctgcttgtctcctactatacctgtctgacacaatatcaccagagcaagccatagacagcctgcgagacctaagaggatccggggcaatacagaccatcaagcaatacaattatcttcatgagtttcgggacaaattagctgcacatctatcatcaaga

SEQ ID NO: 18 - DNA, 496 bp, homo sapiens
cgctctcgtttcattttctgcagcgcgccacgaggatggcccacaagcagatctactactcggacaagtacttcgacgaacactacgagtaccggcatgttatgttacccagagaactttccaaacaagtacctaaaactcatctgatgtctgaagaggagtggaggagacttggtgtccaacagagtctaggctgggttcattacatgattcatgagccagaaccacatattcttctctttagacgacctcttccaaaagatcaacaaaaatgaagtttatctggggatcgtcaaatctttttcaaatttaatgtatatgtgtatataaggtagtattcagtgaatacttgagaaatgtacaaatctttcatccatacctgtgcatgagctgtattcttcacagcaacagagctcagttaaatgcaactgcaagtaggttactgtaagatgtttaagataaaagttcttccagtcagtttttctcttaagtgcct

SEQ ID NO: 19 - DNA, 390 bp, homo sapiens
gaaggtgtggttttcatttctcagtcaccaacagatgaataattatgcttaataataaagtatttattaagactttcttcagagtatgaaagtacaaaaagtctagttacagtggatttagaatatatttatgttgatgtcaaacagctgagcaccgtagcatgcagatgtcaaggcagttaggaagtaaatggtgtcttgtagatatgtgcaaggtagcatgatgagcaacttgagtttgttgccactgagaagcaggcgggttgggtgggaggaggaagaaagggaagaattaggtttgaattgctttttaaaaaaaaaagaaaagaaaaagacagcatctcactatgttgccaaggctcatcttgagaagcaggcgggttgggtggg

SEQ ID NO: 20 - DNA, 338 bp, homo sapiens
  misc_feature (46)..(46): n is a, c, g, or t
ccttttcacccttgcttaatagccagagctgtttcatgcctggggnacacacaattctaatgctggactttttcctgggtcatgctgcaacactgatgtcagagcatgtttttnaatgtnctgtggncaggggcagtgattattcngggtgtggataatgtaagaagttacagcagagctccattctaaggcacttggctctcagttttctcagagtgaacatgcctcgtagcttgggtcctatggcaggagtgcaataggacatggatatgcatcacctgttctataaaactggttgctggctgggngcggtggctcaactcgtataatcccaacac

SEQ ID NO: 21 - DNA, 467 bp, homosapiens
tataggatattcccaagccgaccttccctccgccttctcccacatgaggccatctcaggagatggagggcttggcccagcgccaagtaaacagggtacctcaagccccatttcctcacactaagagggcagactgtgaacttgactgggtgagacccaaagcggtccctgtccctctagtgccttctttagaccctcgggccccatcctcatccctgactggccaaacccttgctttcctgggcctttgcaagatgcttggttgtgttgaggtttttaaatatatattttgtactttgtggagagaatgtgtgtgtgtggcagggggccccgccagggctggggacagagggtgtcaaacattcgtgagctggggactcagggaccggtgctgcaggagtgtcctgcccatgccccagtcggccccatctctcatccttttggataagtttctattctgtcagtgtt

SEQ ID NO: 22 - DNA, 523 bp, homo sapiens
aacagagatgtcccccagggagcacatcaagggcaaagagaccaccccctctagcctagcagtgacccagaccatggccaccaaagctcccgagtgtgtggaggacccagatatggcaaaccagaggaagactgccctggagttctgtggagagacttggagctctctctgcacattcttcctcagcatagtgcaggacacgtcatgctaatgaggtcaaaagagaacgggttcctttaagagatgtcatgtcgtaagtccctctgtatactttaaagctctctacagtccccccaaaatatgaacttttgtgcttagtgagtgcaacgaaatatttaaacaagttttgtattttttgcttttgtgttttggaatttgccttatttttcttggatgcgatgttcagaggctgtttcctgcagcatgtatttccatggcccacacagctatgtgtttgagcagcgaagagtctttgagctgaatgagccagagtgataatttcagtgcaacgaactttctgctg

SEQ ID NO: 23 - DNA, 569 bp, homo sapiens
ggaaccatggacacagtttctctcagtgggactattccagttcaaatgcttttggaaattggtttggacaaactaaagaaagattatatcagttttttcataggtcaggaacttgcatctttgaatcatttggaatacttcattgctccatcagtagatatacaagaacaggtttatcgtgtccaaaaactccaccatattctagaaatattagtcagttgcatgcctttcattaaatctcaacatgaactcctcttttctttaacacagatctgcataaagtattacaaacaaaatcctcttgatgagcaacacatttttcagctgccagtcagaccaactgctgtaaagaacttatatcaaagtgagaagccacagaaatggagagtggaaatatatagtggtcaaaagaagattaagacagtttggcaactgagtgacagctcacccataggccatctgaattttcacaaacctgatttttcggaattaacactaagcggtagcctggaagaaaggatattctttactaacatggttacctgcagccaggtgcatttcaagtgaag

SEQ ID NO: 24 - DNA, 472 bp, homosapiens
tcaattgacttctgttccttgcttttagttttgatagaagggaagacctgcagtgcacggtttcttccaggctgaggtacctggatcttgggttcttcactgcagggacccagacaagtggatctgcttgccagagtcctttttgcccctccctgccacctccccgtgtttccaagtcagctttcctgcaagaagaaatcctggttaaaaaagtcttttgtattgggtcaggagttgaatttggggtgggaggatggatgcaactgaagcagagtgtgggtgcccagatgtgcgctattagatgtttctctgataatgtccccaatcataccagggagactggcattgacgagaactcaggtggaggcttgagaaggccgaaagggcccctgacctgcctggcttccttagcttgcccctcagctttgcaaagagccaccctaggccccagctgaccgcatgggtgtgagcc

SEQ ID NO: 25 - DNA, 353 bp, homosapiens
aattcgagtctatacaaaagccttgagttctttagaactcagtagccatcttgcaaaagatcttctggttctattgaatgagattctggagcaagtaaaagataggacatgtctgagagctttggagaaaatcaagattcagttagaaaaaggaaataaagaatttggtgaccaagctgaagcagcacaggatgccaccttgactacaactactttccaaaatgaagatgaaaagaataaagaagtatatatgactccactcaggggtgtaaaagcaacccaagcatcaaagtctactcagctaaagactaacagaggacagagaaaagtgacagtttcagctaggacgaaca

SEQ ID NO: 26 - DNA, 476 bp, homosapiens
aaaacatgggcgcttacactgttgctgctgcctctacgttcaatggcttccagaggccgacgatctactatgtgatgtcagggcctgcgtggcaactcatgcagcaattccagaaccccgacttcccacccgaagtagaggaacaggatgccagcaccctgcctgtgtcttgtgcctgggagagtgggatgaaacgccacagagcagcctgtgcttcggctagtattaatgtgtagatagcactctggtagctgttaactgcaagtttagcttgaattaagggatttggggggaccatgtaacttaattactgctagttttgaaatgtctttgtaagagtagggtcgccatgatgcagccatatggaagactaggatatgggtcacacttatctgtgttcctatggaaactatttgaatatttgttttatatggatttttattcactcttcagacacgctactcaagagtgcccct

SEQ ID NO: 27 - DNA, 375 bp, homo sapiens
ttttaccttggatgctgacttctaaatgaactgaagatgtgcccttacttggctgattttttttttccatctcataagaaaaatcagctgaagtgttaccaactagccacaccatgaattgtccgtaatgttcattaacagcatctttaaaactgtgtagctacctcacaaccagtcctgtctgtttatagtgctggtagtatcaccttttgccagaaggcctggctggctgtgacttaccatagcagtgacaatggcagtcttggctttaaagtgaggggtgaccctttagtgagcttagcacagcgggattaaacagtcctttaaccagcacagccagttaaaagatgcagcctcactgcttcaacgcagatt

SEQ ID NO: 28 - DNA, 525 bp, homo sapiens
  misc_feature (44)..(44): n is a, c, g, or t
cgatgcaagtgtttctgttctgggaggtattggagggaaaaaancaagcaggatggctggaacactgtactgaggaatgaatagaaaggcttccagatgtctaaaagattctttaaactactgaactgttacctaggttaacaaccctgttgagtatttgctgtttgtccagttcaggaatttttgttttgttttgtctatatgtgcggcttttcagaagaaatttaatcagtgtgacagaaaaaaaaatgttttatggtagcttttactttttatgaaaaaaaaattatttgccttttaaattcttttcccccatccccctccaaagtcttgatagcaagcgttattttgggggtagaaacggtgaaatctctagcctctttgtgtttttgttgttgttgttgttgttgttttatataatgcatgtattcactaaaataaaatttaaaaaactcctgtcttgctagacaaggttgctgttgtgcagtgtgcctgtcactactggtctgtactccttggatttgc

SEQ ID NO: 29 - DNA, 268 bp, homo sapiens
tgccctgaccccgatcagttaaggagctgtgcaataaccttcctagtacctgagtgagtgtgtaacttattgggttggcgaagcctggtaaagctgttggaatgagtatgtgattctttttaagtatgaaaataaagatatatgtacagacttgtattttttctctggtggcattcctttaggaatgctgtgtgtctgtccggcaccccggtaggcctgattgggtttctagtcctccttaaccacttatctcccatatgagagtgtg

SEQ ID NO: 30 - DNA, 480 bp, homo sapiens
gcagctccagctcagagcagtgccagccccaccgcaactgcacggccctgggcctggccctcaatgtgccaggctcttcctcccatgacaccctgtgcaccagctgcactggcttccccctcagcaccagggtaccaggagctgaggagtgtgagcgtgccgtcatcgactttgtggctttccaggacatctccatcaagaggctgcagcggctgctgcaggccctcgaggccccggagggctggggtccgacaccaagggcgggccgcgcggccttgcagctgaagctgcgtcggcggctcacggagctcctgggggcgcaggacggggcgctgctggtgcggctgctgcaggcgctgcgcgtggccaggatgcccgggctggagcggagcgtccgtgagcgcttcctccctgtgcactgatcctggccccctcttatttattctacatccttggcaccccacttgcactgaaagaggc

SEQ ID NO: 31 - DNA, 562 bp, homo sapiens
gaagaaccatcgaaacctgtttgttcccagcccacccccagtggatgggatgcataatgccagcaagttttgtttaacagcaaaaaaggaagattaatgcaggtgttatagaagccagaagagaaactgtgtcaccctaaagaagcatataatcatagcattaaaaatgcacacattactccaggtggaaggtggcaattgctttctgatatcagctcgtttgatttagtgcaaaaatgttttcaagactatttaatggatgtaaaaaagcctatttctacattataccaactgagaaaaaaatggtcggtaaagtgttctttcataataaataatcaagacatggtcccatttgcaggaaaagtgcagactctgagtgttccagggaaacacatgctggacatcccttgtaacccggtatgggcgcccctgcattgctgggatgtttctgcccacggttttgtttgtgcaataacgttatcacatttctaatgaggattcacattaatataatataaaataaataggtcagttactggtctctttctgccgaatgttatgt

SEQ ID NO: 32 - DNA, 329 bp, homo sapiens
ctcttcgagagaacctgtcgccagtatgacaagctgcgtaagcgggaggccttcctggagcagttccgcaaggaggacatgttcaaggacaactttgatgagatggacacatccagggagattgtgcagcagctcatcgatgagtaccatgcggccacacggccagactacatctcctggggcacccaggagcagtgagtcccccaggacaggggaccctcatctgccttactggttggcccaagccctgcctgactgaccaccccctcagagcacagatcagggacctcacgcatctctttctcatatacatggactctctgttggcc